(19) United States
(12) Patent Application Publication
(10) Pub. No.: US 2019/0064191 A1
(43) Pub. Date: Feb. 28, 2019

(54) MEANS AND METHODS FOR DIFFERENTIATING BETWEEN HEART FAILURE AND PULMONARY DISEASE IN A SUBJECT

(71) Applicants: METANOMICS GMBH, Berlin (DE); RUPRECHT-KARLS-UNIVERSITAET HEIDELBERG, Heidelberg (DE)

(72) Inventors: Philipp SCHATZ, Berlin (DE); Henning WITT, Berlin (DE); Erik PETER, Potsdam (DE); Martin DOSTLER, Henningsdorf (DE); Susan CARVALHO, Ludwigshafen (DE); Philipp TERNES, Berlin (DE); Philipp MAPPES, Berlin (DE); Jenny J. FISCHER, Berlin (DE); Hugo KATUS, Heidelberg (DE); Tanja WEIS, Wiesenbach (DE); Norbert FREY, Kronshagen (DE)

(21) Appl. No.: 16/075,348

(22) PCT Filed: Feb. 3, 2017
(86) PCT No.: PCT/EP2017/052452

§ 371 Date: Aug. 3, 2018

(30) Foreign Application Priority Data
Feb. 4, 2016 (EP) 16154325.1

(51) Int. Cl. G01N 33/92

(52) U.S. Cl. CPC G01N 33/92; G01N 2800/325; G01N 2800/12; G01N 2333/58

(57) ABSTRACT

The present invention relates to the field of diagnostic methods. Specifically, the present invention contemplates a method for differentiating in a subject between heart failure and pulmonary disease based on determining the amounts of at least one biomarker. The invention also relates to tools for carrying out the aforementioned methods, such as diagnostic devices.

The present invention relates to the field of diagnostic methods. Specifically, the present invention contemplates a method for differentiating in a subject between heart failure and pulmonary disease based on determining the amounts of at least one biomarker. The invention also relates to tools for carrying out the aforementioned methods, such as diagnostic devices.

Heart failure is a severe problem in modern medicine. The impaired function of the heart can give rise to life-threatening conditions and results in discomfort for the patients suffering from heart failure. Heart failure can affect the right or the left heart ventricle, respectively, and can vary in strength. A classification system was originally developed by the New York Heart Association (NYHA). According to the classification system, the mild cases of heart failure are categorized as class I cases. These patients only show symptoms under extreme exercise. The intermediate cases show more pronounced symptoms already under less exercise (classes II and III) while class IV, shows already symptoms at rest (New York Heart Association. Diseases of the heart and blood vessels. Nomenclature and criteria for diagnosis, 6^{th} ed. Boston: Little, Brown and co, 1964; 114).

The prevalence of heart failure steadily increases in the population of the Western developed countries over the last years. One reason for said increase can be seen in an increased average life expectation and improved survival after myocardial infarction due to modern medicine.

Diagnosis of heart failure today relies predominantly on clinical symptoms, imaging modalities and the biomarkers, brain natriuretic peptide (BNP) and amino-terminal pro-brain natriuretic peptide (NT-proBNP). Recently, metabolic biomarkers for diagnosing heart failure and/or for monitoring heart failure progression or regression have been reported (see WO2011/092285 and WO2013/014286, respectively).

A typical symptom for heart failure is shortness of breath (dyspnea). Diagnosis of shortness of breath is mostly performed using electrocardiogram and chest radiographs. Shortness of breath, however, may have various non-cardiac causes such as pulmonary diseases. In light of a potential cardiac cause of the symptom, it is highly advisable to differentiate between heart failure and pulmonary disease. The correct identification of the cause of shortness of breath allows for the initiation of suitable treatment measures.

There is a long-standing need for means and methods allowing a differentiation between heart failure and pulmonary disease. These means and methods shall allow a reliable efficient differentiation.

Thus, the technical problem underlying the present invention must be seen as the provision of means and methods for complying with the aforementioned needs.

The technical problem is solved by the embodiments characterized in the claims and herein below.

Therefore, the present invention relates to a method for differentiating between heart failure and pulmonary disease in a subject comprising the steps of:
a) determining in a sample of a subject the amounts of, in particular, at least three biomarkers; and
b) comparing i) the amounts as determined in step a) to reference amounts or ii) a score based on the amounts of the at least three biomarkers to a reference score, whereby it is differentiated in a subject between heart failure and pulmonary disease.

The present invention relates to a method for differentiating between heart failure and pulmonary disease in a subject comprising the steps of:
a) determining in a sample of a subject the amount of at least one biomarker selected from the biomarkers shown in column 1 of Table 1; and
b) comparing the amount as determined in step a) to a reference amount, whereby it is differentiated in a subject between heart failure and pulmonary disease.

The method as referred to in accordance with the present invention includes a method which essentially consists of the aforementioned steps or a method which includes further steps. For example, the method may comprise the determination of the amounts of additional markers such as a protein marker as described herein below. However, it is to be understood that the method, in a preferred embodiment, is a method carried out in vitro or ex vivo, i.e. not practised on the human or animal body. The method, preferably, can be assisted by automation.

The phrase “differentiating between heart failure and pulmonary disease” as used herein preferably means distinguishing between a subject who suffers from pulmonary disease and a subject who suffers from heart failure. The expression preferably includes differentially diagnosing a pulmonary disease and heart failure, even more preferably differentially diagnosing a pulmonary disease and heart failure with reduced ejection fraction. The term “differentiating between heart failure and pulmonary disease”, in particular, means to differentiate between pulmonary disease and heart failure as the cause for shortness of breath (for an explanation of the term “shortness of breath” see below). Thus, it shall be assessed whether shortness of breath in a subject is caused by pulmonary disease or heart failure. As will be understood by those skilled in the art, such an assessment, although preferred to be, may usually not be correct for 100% of the investigated subjects. The term, however, requires that a statistically significant portion of subjects can be correctly assessed and, thus, distinguished. Whether a portion is statistically significant can be determined without further ado by the person skilled in the art using various well known statistic evaluation tools, e.g., determination of confidence intervals, p-value determination, Student's t-test, Mann-Whitney test, etc. Details are found in Dowdy and Wearden, Statistics for Research, John Wiley & Sons, New York 1983. Preferred confidence intervals are at least 50%, at least 60%, at least 70%, at least 80%, at least 90% or at least 95%. The p-values are, preferably, 0.2 or lower, 0.1 or lower, or 0.05 or lower. Preferably, the differentiation is based on the biomarkers to be determined in the method of the present invention, i.e. on the at least three biomarkers as specifically referred to in step a) of the method of the present invention and, if determined, on NT-proBNP or BNP.

In an embodiment, the differentiation as referred to herein may comprise further steps such as the verification of the differentiation. Accordingly, the differentiation as referred to herein, preferably, shall assist the differentiation between heart failure and pulmonary disease.

The term “subject” as used herein relates to animals and, preferably, to mammals. More preferably, the subject is a primate and, most preferably, a human. The subject may be a female or, in particular, a male subject.

The subject to be tested, preferably, is suspected to suffer heart failure or pulmonary disease. A subject suspect to suffer from heart failure or pulmonary disease preferably suffers from shortness of breath (herein also referred to as dyspnea).

In a preferred embodiment, the shortness of breath is chronic shortness of breath.

In another preferred embodiment, the shortness of breath is acute shortness of breath.

Thus, it is contemplated that the subject to be tested suffers from shortness of breath. Accordingly, the present invention relates to a method for differentiating between heart failure and pulmonary disease in a subject suffering from shortness of breath. In particular, it is differentiated between heart failure and pulmonary disease as the cause for the shortness of breath.

The term “shortness of breath” is well known in the art. As used herein, the term preferably refers to an impaired respiration. Preferably, the impaired respiration results in an elevated respiratory volume and/or in an elevated respiratory frequency. It is a subjective experience of breathing discomfort that comprises qualitatively distinct sensations that vary in intensity. Distinct sensations include e.g. chest tightness and air hunger. Shortness of breath can occur at an oxygen saturation level below the normal oxygen saturation level of at least 95%. Shortness of breath can result in hyperventilation. As set forth above, shortness of breath encompasses acute shortness of breath and chronic shortness of breath. Acute shortness of breath is preferably a non-permanently occurring shortness of breath, whereas chronic shortness of breath is preferably a permanently occurring shortness of breath. Acute shortness of breath is, usually, progressively worsening. Preferably, acute shortness of breath persists no longer than one week from the acute onset, whereas chronic dyspnea is characterized as persisting for a period of time of more than one week, in particular of more than one month.

Preferably, the subject is an adult. More preferably, the subject is older than 40 years of age, and most preferably older than 50 years of age. Further, it is envisaged that the subject is older than 54 years of age, but younger than 61 years of age.

In one embodiment, the subject is older than 54 years of age, in particular older than 60 years of age, or even older than 70 years of age.

In another embodiment, the subject has a body mass index (BMI) of more than 26.8 kg/m², in particular of more than 28.0 kg/m², or even more than 30.0 kg/m².

Preferably, the subject, however, is besides the aforementioned diseases and disorders apparently healthy. Also preferably, the subject shall not suffer from apoplex (stroke), myocardial infarction within the last 4 month before the sample has been taken or from acute or chronic inflammatory diseases and malignant tumors. Furthermore, the subject is preferably in stable medications within the last 4 weeks before the sample was taken.

In a preferred embodiment, the subject to be tested does not suffer from impaired renal function. However, it is also contemplated that the subject suffers from impaired renal function. Renal function can be assessed e.g. by determining the glomerular filtration rate (GFR). Preferably, a subject suffers from impaired renal function if the GFR is below 60 mL/min/1.73 m², or in particular below 50 mL/min/1.73 m². Preferably, a subject does not suffer from impaired renal function if the GFR is above 60 mL/min/1.73 m², or in particular above 70 mL/min/1.73 m².

More preferably, the subject who does not suffer impaired renal disease does not suffer from chronic kidney disease stages 3 to 5 (for the classification, see e.g. National Kidney Foundation, 2002. K/DOQI clinical practice guidelines for chronic kidney disease: evaluation, classification, and stratification. Am. J. Kidney Dis. 39, 51-266 which herewith is incorporated by reference in its entirety.)

GFR may be accurately calculated by comparative measurements of substances in the blood and urine, or estimated by formulas using just a blood test result (eGFR). Usually these estimates are used in clinical practice in particular in elderly and sick patients where reliable urine collections are difficult. These tests are important in assessing the excretory function of the kidneys, for example in grading of chronic renal insufficiency.

eGFR is associated with GFR via a large clinical study (National Kidney Foundation (February 2002). “K/DOQI clinical practice guidelines for chronic kidney disease: evaluation, classification, and stratification”. American Journal of Kidney Diseases 39 (2 Suppl 1): S1-266. doi:10.1016/S0272-6386(02)70081-4. PMID 11904577). For clinical assessment scales of eGFR and GFR can be used interchangeably.

The term “pulmonary disease” is well understood by the skilled person. As used herein, the term refers to any kind of lung disease. In particular, the term refers to any kind of lung disease which causes shortness of breath. Such diseases are well known in the art. Preferably, the term pulmonary disease includes COPD (chronic obstructive pulmonary disease), pulmonary fibrosis, asthma, pneumothorax, pulmonary infection, acute respiratory distress syndrome, pulmonary edema, lung injury or hemorrhage, respiratory failure and pulmonary embolism. In particular, the term includes COPD, asthma and pulmonary fibrosis.

The term “heart failure” is well known in the art. As used herein, it relates to an impaired function of the heart. It is a progressive disorder in which the heart fails to pump oxygenated blood at a rate sufficient to meet the metabolic needs of the tissues. Preferably, the term heart failure as used herein relates to congestive heart failure (CHF).

Heart failure is the final common stage of many cardiovascular diseases and is defined as a clinical syndrome in which patients in the final stage show typical signs and symptoms of effort intolerance and/or fluid retention resulting from an abnormality of cardiac structure or function. As set forth above, a typical symptom of heart failure is shortness of breath.

The impaired function of the heart can be a systolic dysfunction resulting in a significantly reduced ejection fraction of blood from the heart and, thus, a reduced blood flow. Specifically, systolic heart failure is characterized by a significantly reduced left ventricular ejection fraction (LVEF), preferably, an ejection fraction of less than 50% (heart failure with reduced ejection fraction, HFrEF). Alternatively, the impairment can be a diastolic dysfunction, i.e. a failure of the ventricle to properly relax. The latter is usually accompanied by a stiffer ventricular wall. The diastolic dysfunction causes inadequate filling of the ventricle and, therefore, results in consequences for the blood flow, in general. Thus, diastolic dysfunction also results in elevated end-diastolic pressures, and the end result is comparable to the case of systolic dysfunction (pulmonary edema in left heart failure, peripheral edema in right heart failure). Heart failure may, thus, affect the right heart (pulmonary circulation), the left heart (body circulation) or both. Techniques for measuring an impaired heart function and, thus, heart failure, are well known in the art and include echocardiography, electrophysiology, angiography. It will be understood that the impaired function of the heart can occur permanently or only under certain stress or exercise conditions. Dependent on the strength of the symptoms, heart failure can be classified as set forth elsewhere herein. Typical symptoms of heart failure include dyspnea, chest pain, dizziness, confusion, pulmonary and/or peripheral edema. It will be understood that the occurrence of the symptoms as well as their severity may depend on the severity of heart failure and the characteristics and causes of the heart failure, systolic or diastolic or restrictive i.e. right or left heart located heart failure. Further symptoms of heart failure are well known in the art and are described in the standard text books of medicine, such as Stedman or Brunnwald.

Two subsets of heart failure with different pathophysiology are described based on a measurement of left ventricular ejection fraction (LVEF), which is the percentage of the total amount of blood in the left ventricle that is pushed out with each heartbeat: Heart failure with reduced left ventricular ejection fraction (HFrEF) and heart failure with preserved left ventricular ejection fraction (HFpEF). Preferably, the term heart failure includes both HFpEF and HFrEF. In a preferred embodiment of the present invention, heart failure is HFrEF.

HFrEF, also known as systolic heart failure, is characterized by reduced heart muscle contraction and emptying of the left ventricle. The expression “reduced left ventricular ejection fraction”, preferably, relates to a left ventricular ejection fraction (LVEF) of lower than 50%.

While there are many causes of HFrEF, the most common is related to ischemic cardiomyopathy resulting from coronary artery disease and prior myocardial infarctions. Ischemic cardiomyopathy (ICMP) occurs when narrowed or blocked coronary arteries restrict blood flow and oxygen supply to the heart tissue, damaging or weakening the heart muscle (loss of functional myocardium). Further, HFrEF may have non-ischemic causes. One of the most common non-ischemic causes of HFrEF is dilatative cardiomyopathy (DCMP, also referred to as dilated cardiomyopathy). DCMP is a condition in which the heart's ability to pump blood is decreased because the heart's main pumping chamber, the left ventricle, is enlarged, dilated and weak. The cause for DCMP can range from heart muscle infection (myocarditis), high blood pressure, heart valve disease, and alcohol abuse to familial (hereditary) forms. Preferably, the terms NICMP (nonischemic cardiomyopathy) and DCMP are used interchangeably herein.

A subject who suffers from HFrEF may, thus, suffer from dilatative cardiomyopathy (DCMP, frequently also referred to as dilated cardiomyopathy) or ischemic cardiomyopathy (ICMP).

Accordingly, the term HFrEF preferably also encompasses ICMP and DCMP. A subject who suffers from ICMP, preferably, has a reduced LVEF and more than 50% coronary stenosis. A subject who suffers from DCMP, preferably, has a reduced LVEF and less than 50% coronary stenosis. In particular, a subject who suffers from DCMP, preferably, has a reduced LVEF, less than 50% coronary stenosis and a left ventricle wall thickness>55 mm. Further, a subject who suffers from DCMP may have a reduced LVEF, less than 50% coronary stenosis and a left ventricular end diastolic diameter of larger than 55 mm.

Symptoms of heart failure are well known in the art and are described above. Asymptomatic heart failure is preferably heart failure according to NYHA class I. A subject with heart failure according to NYHA class I has no limitation of physical activity and ordinary physical activity does not cause undue breathlessness, fatigue, or palpitations. Symptomatic heart failure is preferably heart failure according to NYHA class II, Ill and/or VI, in particular according to NYHA class II and/or III. A subject with heart failure according to NYHA class II or III has a slight (NYHA class II) or marked (NYHA class III) limitation of physical activity, is comfortable at rest but ordinary (NYHA class II) or less than ordinary (NYHA class III) physical activity results in undue shortness of breath, fatigue, or palpitations.

By carrying out the steps of the method of the present invention, it could be also assessed whether shortness of breath in a subject is caused by heart failure by other causes. In this context, other causes include pulmonary disease, obesity, high body weight, an untrained or poorly trained physical condition of the subject, psychological conditions such as anxiety states. Based on the determination of the amounts of the biomarkers as referred to herein in a sample from a subject suffering from shortness of breath, it can be assessed whether a subject suffers from heart failure, or not. If the subject suffers from heart failure, is is likely that heart failure is the cause for shortness of breath. If the subject does not suffer from heart failure, it is likely that the shortness of breath has causes other than heart failure (such as pulmonary disease).

The method of the present invention envisages the determination of the at least one biomarker shown in column 1 of Table 1 in the Examples section. In particular the present invention envisages the determination of the amount of at least three biomarkers, i.e. of a combination of at least three biomarkers. Preferred combinations are described elsewhere herein. The term “biomarker” as used herein refers to a molecular species which serves as an indicator for a disease or effect as referred to in this specification. Said molecular species can be a metabolite itself which is found in a sample of a subject. Moreover, in certain cases the biomarker may also be a molecular species which is derived from said metabolite. In such a case, the actual metabolite will be chemically modified in the sample or during the determination process and, as a result of said modification, a chemically different molecular species, i.e. the analyte, will be the determined molecular species. It is to be understood that in such a case, the analyte represents the actual metabolite and has the same potential as an indicator for the respective medical condition. In an embodiment of the present invention the triacylglyceride(s) will be determined as such, as disclosed elsewhere herein. In an embodiment of the present invention the phosphatidylcholine(s) will be determined as such, as disclosed elsewhere herein. In an embodiment of the present invention the ceramide(s) will be determined as such, as disclosed elsewhere herein. In an embodiment of the present invention the sphingomyelin(s) will be determined as such, as disclosed elsewhere herein. In an embodiment of the present invention the cholesterylester(s) will be determined as such, as disclosed elsewhere herein. Preferably, the at least three biomarkers to be determined in accordance with the present invention are metabolite biomarkers (with the exception of the further markers BNP and NT-proBNP which are peptides/protein markers, see below). The term “metabolite” is well known in art. Preferably, the metabolite in accordance with the present invention is a small molecule compound.

In the method according to the present invention the amount of at least one biomarker shown in column 1 of Table 1 shall be determined. In particular, at least the amounts of at least three biomarkers shall be determined (i.e. of at least three metabolite biomarkers). The term “at least three biomarkers” as used herein, means three or more than three. Accordingly, the amounts of three, four, five, six, seven, eight, nine, ten or even more biomarkers may be determined (and compared to a reference). Preferably, the amounts of three to ten biomarkers, in particular metabolite biomarkers, are determined (and compared to a reference).

Preferably, the biomarker(s) to be determined is a lipid biomarker (are lipid metabolite biomarkers). Thus, the biomarkers are preferably metabolite biomarkers (i.e. lipid biomarkers). More preferably, the biomarker(s) to be determined is (are) selected from the group consisting of at least one sphingomyelin biomarker, at least one triacylglyceride biomarker, at least one cholesterylester biomarker, at least one phosphatidylcholine biomarker, and/or at least one ceramide biomarker. Thus, the method of the present invention envisages the determination of one or more sphingomyelin biomarker, one or more triacylglyceride,biomarker, one or more cholesterylesters biomarker, one or more phosphatidylcholine biomarker, and/or one or more ceramide biomarker. Furthermore, the amount of glutamic acid may be determined. In an embodiment, the amounts of i) at least one one sphingomyelin biomarker, ii) at least one triacylglyceride biomarker, and iii) at least one cholesterylester biomarker or at least one phosphatidylcholine biomarker are determined.

The at least three biomarkers to be determined preferably belong to at least three of the different compound classes referred to above. For example, one of the at least three biomarkers is a triacylglycerid biomarker, one is a sphingomyelin biomarker, and one is a cholesterylester biomarker. However it is also envisaged that the at least three biomarkers belong to a single compound class, or to two compound classes. In addition, the amount of at least one biomarker selected from the biomarkers shown in column 1 of Table 1 can be determined and compared to a reference amount for differentiating between heart failure and pulmonary disease. For example the amount of SM23 or OSS2 can be determined. In addition, the amounts of both markers, i.e. of SM23 and OSS2, can be determined.

Preferred biomarkers to be determined in accordance with the present invention are disclosed in column 1 of Table 1 of the examples section.

Thus, the at least one triacylglycerid biomarker is preferably selected from the group consisting of OSS2, SOP2, SPP1, SSP2, SSS, PPO1, and PPP, more preferably selected from the group consisting of OSS2, SOP2, SPP1, SSP2, PPO1 and PPP, even more preferably selected from OSS2, SOP2, SPP1, SSP2 and PPO1, in particular OSS2 and SOP2, and most preferably OSS2. Preferably, one, two or three, or more triacylglyceride biomarker are determined.

The at least one sphingomyelin biomarker is preferably selected from the group consisting of SM10, SM18, SM2, SM21, SM23, SM24, SM28, SM29, SM3, SMS, SM8, and SM9 (in particular from SM10, SM18, SM21, SM23, SM24 and SM28). More preferably, the at least one sphingomyelin biomarker is selected from the group consisting of SM23, SM24, and SM18. Even more preferably, the at least one sphingomyelin biomarker is SM18 or SM24, and most preferably SM23. Preferably, one, two, three, four or more sphingomyelin biomarker are determined.

The at least one ceramide biomarker is preferably selected from the group consisting of Cer(d16:1/24:0), Cer(d17:1/24:0), Cer(d18:1/23:0), Cer(d18:1/24:1), and Cer(d18:2/24:0). Preferably, the amounts of one or two, or more ceramide biomarker are determined. More preferably, the at least one ceramide biomarker is selected from the group consisting of Cer(d16:1/24:0), and Cer(d18:1/24:1). Even more preferably, the at least one ceramide biomarker is Cer(d16:1/24:0).

The at least one phosphatidylcholine biomarker is preferably PC4 or PC8. Preferably, the amount of one phosphatidylcholine biomarker is determined, in particular PC4. However, it is also envisaged to determine both biomarker PC4 and PC8.

The at least one cholesterylester biomarker is preferably cholesterylester C18:0 or cholesterylester C18:2. Preferably, the amount of one cholesterylester biomarker is determined, in particular cholesterylester 018:2. However, it also envisaged to determine both cholesterylester C18:0 and cholesterylester C18:2. Preferably, the cholesterylester biomarker is not cholesterylester C18:1.

Accordingly, the biomarker(s) as referred to in step a) of the method of the present invention is (are) preferably selected from the group of biomarkers consisting of OSS2, SOP2, SPP1, SSP2, SSS, PPO1, PPP, SM10, SM18, SM2, SM21, SM23, SM24, SM28, SM29, SM3, SM5, SM8, SM9, Cer(d16:1/24:0), Cer(d17:1/24:0), Cer(d18:1/23:0), Cer(d18:1/24:1), Cer(d18:2/24:0), PC4, PC8, cholesterylester C18:0, cholesterylester C18:2, and glutamic acid. The aforementioned biomarkers are metabolite biomarkers.

If the amount of at least one biomarker is determined the biomarker is preferably selected from SM23, PC4, OSS2, and cholesterylester C18:2. In particular, the amount of SM23 or OSS2 is determined (and compared to a reference). Further, it is envisaged that the amounts of SM23 and OSS2 are determined.

Biomarker Definitions

In accordance with the present invention, the amount of at least one metabolite biomarker, in particular the amounts of at least three metabolite biomarkers selected from the group consisting of OSS2, SOP2, SPP1, SSP2, SSS, PPO1, PPP, SM10, SM18, SM2, SM21, SM23, SM24, SM28, SM29, SM3, SMS, SM8, SM9, Cer(d16:1/24:0), Cer(d17:1/24:0), Cer(d18:1/23:0), Cer(d18:1/24:1), Cer(d18:2/24:0), PC4, PC8, cholesterylester C18:0, cholesterylester C18:2 and glutamic acid shall be determined.

A preferred definition of these biomarkers is provided in Table 1 of the Examples section. Accordingly, the biomarkers are preferably defined as follows:

Triacylglyceride (TAG) Biomarkers

The biomarkers OSS2, SOP2, SPP1, SSP2, SSS, PPO1, and PPP are triacylglycerides (TAG: triacylglyceride).
OSS2 is TAG(C18:1, C18:0, C18:0)
PPO1 is TAG(C16:0, C16:0, C18:1)
PPP is TAG(C16:0, C16:0, C16:0)
SOP2 is TAG(C18:0, C18:1, C16:0)
SPP1 is TAG(C18:0, C16:0, C16:0)
SSP2 is TAG(C18:0, C18:0, C16:0)
SSS is TAG(C18:0, C18:0, C18:0)

The triacylglyceride TAG(Cx¹:y¹, Cx²:y², Cx³:y³) is preferably denoted to mean that the triacylglyceride comprises three fatty acid ester residues, wherein one fatty acid ester residue is Cx¹:y¹ which means that this residue comprises x¹ carbon atoms and y¹ double bonds, wherein one fatty acid ester residue is Cx²:y²which means that this residue comprises x² carbon atoms and y² double bonds, and wherein one fatty acid ester residue is Cx³:y³ which means that this residue comprises x³ carbon atoms and y³ double bonds. Preferably, any of these fatty acid ester residues may be attached to any former hydroxyl groups of the glycerol.

For example, SOP2 comprises three fatty acid ester residues, wherein one fatty acid ester residue is C18:0 which means that this residue comprises 18 carbon atoms and 0 double bonds, wherein one fatty acid ester residue is C18:1 which means that this residue comprises 18 carbon atoms and 1 double bond, and wherein one fatty acid ester residue is C16:0 which means that this residue comprises 16 carbon atoms and 0 double bonds. Preferably, any of these fatty acid ester residues may be attached to any former hydroxyl groups of the glycerol.

Ceramide (CER) Biomarkers

The biomarkers Cer(d16:1/24:0), Cer(d17:1/24:0), Cer(d18:1/23:0), Cer(d18:1/24:1), and Cer(d18:2/24:0) are ceramides.

The ceramide CER(dx¹:y¹/x²:y²) is preferably denoted to mean that the ceramide comprises the “sphingosine-backbone” dx¹:y¹, wherein x¹ denotes the number of carbon atoms and y¹ the number of double bonds, and a “fatty acid amid” residue x²:y², wherein x² denotes the number of carbon atoms and y² the number of double bonds thereof. Preferably, “d” indicates that the backbone comprises two hydroxyl groups.
Cer(d16:1/24:0) is Ceramide (d16:1124:0)
Cer(d17:1/24:0) is Ceramide (d17:1124:0)
Cer(d18:1/23:0) is Ceramide (d18:1123:0)
Cer(d18:1/24:1) is Ceramide (d18:1124:1)
Cer(d18:2/24:0) is Ceramide (d18:2124:0)

For example, the ceramide Cer(d16:1/24:0) is preferably denoted to mean that the ceramide comprises the “sphingosine-backbone” d16:1, comprising 16 carbon atoms and 1 double bond, and a “fatty acid amid” residue 24:0 comprising 24 carbon atoms and 0 double bonds.

Sphingomyelin (SM) Biomarkers

SM is the abbreviation for Sphingomyelin. As set forth herein below, the amounts of the sphingomyelin biomarkers SM10, SM18, SM2, SM21, SM23, SM24, SMS, and SM9 may be determined by determining the amount of a single sphingomyelin species in the sample, or by determining the total amount of two (or even three) sphingomyelin species which have the same or essentially the same molecular weight (see Table 1 in the Examples section). For example, for SM10 the amount of Sphingomyelin(d18:1/18:0) can be determined, or the total amount of Sphingomyelin(d18:1/18:0) and Sphingomyelin(d16:1/20:0). Whether the amount of a single species or of two (or three) species is determined may depend on the assay used for the determination. For example, the amount of a single species is determined, if the assay underlying the determination step (step a.) allows for the determination of a single species. The total amount of two (or three) species may be determined, if the assay underlying the determination step allows for the determination of the total amount of the two (or three) species only (rather than for the determination of the single species), in other words the assay is not capable of differentially determining the amounts of each of the two (or three) species. E.g. if the determination of the amount of a biomarker comprises mass spectrometry, the determination of the amount of the biomarker is preferably based on a single peak in a mass spectrum for the two (or three) species since the peaks of the species overlap. This is taken into account by the skilled person. The determination of a single species and the determination of two (or three) species as described herein below (or in Table 1) shall allow for a reliable differentiation. Moreover, the determination of the total amount of two (or three) species having the same molecular weight is less complex (as compared to the determination of a single species). The same applies to the determination of PC8 (see below).

In Table 1 of the Examples section, the preferred Sphingomyelin species for SM10, SM18, SM2, SM21, SM23, SM24, SM5, and SM9 are listed (the species are referred to as “analyte” in this table).

For SM10, SM2, SM24, SM5, and SM9 two analytes are listed (analyte 1 and analyte 2). The said biomarkers preferably refer to Analyte 2 (for example, for SM2: SM(d16:1/16:0)), more preferably to Analyte 1 (for example, for SM2: SM(d18:1/14:0)) and most preferably to Analyte 1 and 2 (for SM2: Sphingomyelin(d18:1/14:0) and Sphingomyelin(d16:1/16:0)). Accordingly, a biomarker for which two species are listed in table 1 is Analyte 1, Analyte 2 or a combination of Analyte 1 and Analyte 2.

The amount of said biomarker is, thus, preferably determined by determining the amount of Analyte 2, or more preferably of Analyte 1 and most preferably by determining the combined (and thus the total) amount of Analyte 1 and Analyte 2. Accordingly, the amount of said biomarker is the amount of Analyte 1 or 2, or the sum of the amounts of Analyte 1 and 2. The same applies to PC8.

For the biomarkers SM18, SM21 and SM 23 three analytes are listed (Analyte 1, 2 and 3). The said biomarkers preferably refer to Analyte 3, more preferably to Analyte 2, even more preferably to Analyte 1, and most preferably to Analyte 1, 2 and 3. Accordingly, a biomarker for which three species are listed in Table 1 is Analyte 1, Analyte 2, or Analyte 3 or a combination of Analyte 1, Analyte 2 and Analyte 3.

If two analytes are listed for a biomarker (Analyte 1 and Analyte 2), it is envisaged that the biomarker is Analyte 1. Alternatively, the biomarker may be Analyte 2. If three analytes are listed for a biomarker (Analyte 1, Analyte 2 and Analyte 3), it is envisaged that that the biomarker is Analyte 1. Alternatively, the biomarker may be Analyte 2. Also, the biomarker may be Analyte 3. Alternatively the biomarker may be a combination of Analyte 1 and Analyte 2. Alternatively the biomarker may be a combination of Analyte 1 and Analyte 3. Alternatively the biomarker may be a combination of Analyte 2 and Analyte 3. Alternatively the biomarker may be a combination of Analyte 1, Analyte 2 and Analyte 3.

The amount of said biomarker is, thus, preferably determined by determining the amount of Analyte 3, or more preferably of Analyte 2, even more preferably of Analyte 1 and most preferably by determining the combined (and thus the total) amount of Analyte 1, Analyte 2, and Analyte 3. Accordingly, the amount of said biomarker is the amount of Analyte 1, 2, or 3, or the sum of the amounts of Analyte 1, 2 and 3.

The following applies in particular:

The biomarker SM10 preferably refers to Sphingomyelin(d18:1/18:0), or more preferably to Sphingomyelin(d18:1/18:0) and Sphingomyelin(d16:1/20:0). Accordingly, the biomarker SM10 is Sphingomyelin(d18:1/18:0), or a combination of Sphingomyelin(d18:1/18:0) and Sphingomyelin(d16:1/20:0).

The amount of SM10 is, thus, preferably determined by determining the amount of Sphingomyelin(d18:1/18:0), or by determining the combined (and thus the total) amount of Sphingomyelin(d18:1/18:0) and Sphingomyelin(d16:1/20:0). Accordingly, the amount of SM10 is the amount of Sphingomyelin(d18:1/18:0), or the sum of the amounts of Sphingomyelin(d18:1/18:0) and Sphingomyelin(d16:1/20:0).

The biomarker SM18 preferably refers to Sphingomyelin(d18:1/21:0), or more preferably to Sphingomyelin(d18:1/21:0), Sphingomyelin(d16:1/23:0) and Sphingomyelin(d17:1/22:0). Accordingly, the biomarker SM18 is Sphingomyelin(d18:1/21:0), or a combination of Sphingomyelin(d18:1/21:0), Sphingomyelin(d16:1/23:0) and Sphingomyelin(d17:1/22:0).

The amount of SM18 is, thus, preferably determined by determining the amount of Sphingomyelin(d18:1/21:0), or by determining the combined (and thus the total) amount of Sphingomyelin(d18:1/21:0) Sphingomyelin(d16:1/23:0) and Sphingomyelin(d17:1/22:0). Accordingly, the amount of SM18 is the amount of Sphingomyelin(d18:1/21:0), or the sum of the amounts of Sphingomyelin(d18:1/21:0), Sphingomyelin(d16:1/23:0) and Sphingomyelin(d17:1/22:0).

The biomarker SM2 preferably refers to Sphingomyelin(d18:1/14:0) or more preferably to Sphingomyelin(d18:1/14:0) and Sphingomyelin(d16:1/16:0). Accordingly, the biomarker SM2 is Sphingomyelin(d18:1/14:0), or a combination of Sphingomyelin(d18:1/14:0) and Sphingomyelin(d16:1/16:0).

The amount of SM2 is, thus, preferably determined by determining the amount of Sphingomyelin(d18:1/14:0), or by determining the combined (and thus the total) amount of Sphingomyelin(d18:1/14:0) and Sphingomyelin(d16:1/16:0). Accordingly, the amount of SM2 is the amount of Sphingomyelin(d18:1/14:0), or the sum of the amounts of Sphingomyelin(d18:1/14:0) and Sphingomyelin(d16:1/16:0).

The biomarker SM21 preferably refers to Sphingomyelin(d17:1/23:0) or more preferably to Sphingomyelin(d17:1/23:0), Sphingomyelin(d18:1/22:0) and Sphingomyelin(d16:1/24:0). Accordingly, the biomarker SM21 is Sphingomyelin(d17:1/23:0), or a combination of Sphingomyelin(d17:1/23:0), Sphingomyelin(d18:1/22:0), and Sphingomyelin(d16:1/24:0).

The amount of SM21 is, thus, preferably determined by determining the amount of Sphingomyelin(d17:1/23:0), or by determining the combined (and thus the total) amount of Sphingomyelin(d17:1/23:0), Sphingomyelin(d18:1/22:0) and Sphingomyelin(d16:1/24:0). Accordingly, the amount of SM21 is the amount of Sphingomyelin(d17:1/23:0), or the sum of the amounts of Sphingomyelin(d17:1/23:0), Sphingomyelin(d18:1/22:0) and Sphingomyelin(d16:1/24:0).

The biomarker SM23 preferably refers to Sphingomyelin(d18:1/23:1) or more preferably to Sphingomyelin(d18:1/23:1), Sphingomyelin(d18:2/23:0) and Sphingomyelin(d17:1/24:1). Accordingly, the biomarker SM23 is Sphingomyelin(d18:1/23:1), or a combination of Sphingomyelin(d18:1/23:1), Sphingomyelin(d18:2/23:0), and Sphingomyelin(d17:1/24:1). Further, in another embodiment, it is envisaged that SM23 is Sphingomyelin (d17:1/24:1).

The amount of SM23 is, thus, preferably determined by determining the amount of Sphingomyelin(d18:1/23:1), or by determining the combined (and thus the total) amount of Sphingomyelin(d18:1/23:1), Sphingomyelin(d18:2/23:0) and Sphingomyelin(d17:1/24:1). Accordingly, the amount of SM23 is the amount of Sphingomyelin(d18:1/23:1), or the sum of the amounts of Sphingomyelin(d18:1/23:1), Sphingomyelin(d18:2/23:0), and Sphingomyelin(d17:1/24:1). Further, it is envisaged, in another embodiment, that the amount of SM23 is determined by determining the amount of Sphingomyelin (d17:1124:1).

In an embodiment, the biomarker SM23 preferably refers to Sphingomyelin(d18:2/23:0). In the embodiment, the biomarker SM23 is, thus, preferably determined by determining the amount of Sphingomyelin (d18:2/023:0).

The biomarker SM24 preferably refers to Sphingomyelin(d18:1/23:0) or more preferably to Sphingomyelin(d18:1/23:0) and Sphingomyelin(d17:1/24:0). Accordingly, the biomarker SM24 is Sphingomyelin(d18:1/23:0), or a combination of Sphingomyelin(d18:1/23:0) and Sphingomyelin(d17:1/24:0).

The amount of SM24 is, thus, preferably determined by determining the amount of Sphingomyelin(d18:1/23:0), or by determining the combined (and thus the total) amount of Sphingomyelin(d18:1/23:0) and Sphingomyelin(d17:1/24:0). Accordingly, the amount of SM24 is the amount of Sphingomyelin(d18:1/23:0), or the sum of the amounts of Sphingomyelin(d18:1/23:0) and Sphingomyelin(d17:1/24:0).

The biomarker SM28 is preferably Sphingomyelin(d18:1/24:0).

The biomarker SM29 is preferably Sphingomyelin(d18:2/17:0).

The biomarker SM3 is preferably Sphingomyelin(d17:1/16:0).

The biomarker SM5 preferably refers to Sphingomyelin(d18:1/16:0) or more preferably to Sphingomyelin(d18:1/16:0) and Sphingomyelin(d16:1/18:0). Accordingly, the biomarker SM5 is Sphingomyelin(d18:1/16:0), or a combination of Sphingomyelin(d18:1/16:0) and Sphingomyelin(d16:1/18:0).

The amount of SM5 is, thus, preferably determined by determining the amount of Sphingomyelin(d18:1/16:0), or by determining the combined (and thus the total) amount of Sphingomyelin(d18:1/16:0) and Sphingomyelin(d16:1/18:0). Accordingly, the amount of SM5 is the amount of Sphingomyelin(d18:1/16:0), or the sum of the amounts of Sphingomyelin(d18:1/16:0) and Sphingomyelin(d16:1/18:0).

The biomarker SM8 is preferably Sphingomyelin(d18:2/18:1).

The biomarker SM9 preferably refers to Sphingomyelin(d18:1/18:1) or more preferably to Sphingomyelin(d18:1/18:1) and Sphingomyelin(d18:2/18:0). Accordingly, the biomarker SM9 is Sphingomyelin(d18:1/18:1), or a combination of Sphingomyelin(d18:1/18:1) and Sphingomyelin(d18:2/18:0).

The amount of SM9 is, thus, preferably determined by determining the amount of Sphingomyelin(d18:1/18:1), or by determining the combined (and thus the total) amount of Sphingomyelin(d18:1/18:1) and Sphingomyelin(d18:2/18:0). Accordingly, the amount of SM9 is the amount of Sphingomyelin(d18:1/18:1), or the sum of the amounts of Sphingomyelin(d18:1/18:1) and Sphingomyelin(d18:2/18:0).

As set forth above, the present invention is based on the amount of at least three biomarkers. In this context, it is noted that SM10, SM18, SM2, SM21, SM23, SM24, SM5, and SM9, and PC8 are considered as single biomarkers regardless whether the determination of these biomarkers is based on the determination of a single, two (or three) species. Thus, at least two further biomarkers have to be determined.

The sphingomyelin SM(dx¹:y¹/x²:y²) is preferably denoted to mean that the sphingomyelin comprises the “sphingosine-backbone” dx¹:y¹, wherein x¹ denotes the number of carbon atoms and y¹ the number of double bonds, and a “fatty acid amide” residue x²:y², wherein x² denotes the number of carbon atoms and y² the number of double bonds thereof.

The Sphingomyelin(d18:2/18:0) is preferably denoted to mean that the sphingomyelin comprises the “sphingosine-backbone”d18:2, which comprises 18 carbon atoms and 2 double bonds, and a “fatty acid amide” residue 18:0, which comprises 18 carbon atoms and 0 double bonds.

Cholesterylester (CE) Biomarkers

CE is the abbreviation for Cholesterylester.

In accordance with the present invention, the determination of two different cholesterylesters is contemplated, i.e. of cholesterylester C18:0 and/or cholesterylester C18:2. The biomarkers are well known in the art. The cholesterylester (Cx¹:y¹) is preferably denoted to mean that the cholesterylester comprises a fatty acid ester residue, wherein said fatty acid ester residue is Cx¹:y¹ which means that this residue comprises x¹ carbon atoms and y¹ double bonds.

For example, cholesterylester C18:0 is denoted to mean that the cholesterylester C18:0 comprises a fatty acid ester residue, wherein said fatty acid ester residue is C18:0 which means that this residue comprises 18 carbon atoms and 0 double bonds.

Phosphatidylcholine (PC) Biomarkers

PC is the abbreviation for Phosphatidylcholine. The Phosphatidylcholine PC (Cx¹:y¹Cx²:y²) is preferably denoted to mean that the phosphatidylcholine comprises two fatty acid ester residues, wherein one fatty acid ester residue is Cx¹:y¹ which means that this residue comprises x¹ carbon atoms and y¹ double bonds, wherein one fatty acid ester residue is Cx²:y², which means that this residue comprises x² carbon atoms and y² double bonds.

For example, phosphatidylcholine (C16:0 018:2) is preferably denoted to mean that the phosphatidylcholine comprises two fatty acid ester residues, wherein one fatty acid ester residue is C16:0 which means that this residue comprises 16 carbon atoms and 0 double bonds, wherein one fatty acid ester residue is C18:2, which means that this residue comprises 18 carbon atoms and 2 double bonds.

The biomarker PC4 is Phosphatidylcholine (C16:0 C18:2). The biomarker PC8 preferably refers to Phosphatidylcholine(C18:0 C18:2) or more preferably to Phosphatidylcholine(C18:0 C18:2) and Phosphatidylcholine(C18:1 C18:1). Accordingly, the biomarker PC8 is Phosphatidylcholine(C18:0 018:2), or a combination of Phosphatidylcholine(C18:0 C18:2) and Phosphatidylcholine(C18:1 C18:1).

The amount of PC8 is, thus, preferably determined by determining the amount of Phosphatidylcholine (C18:0 C18:2), or by determining the combined (and thus the total) amount of Phosphatidylcholine(C18:0 C18:2) and Phosphatidylcholine(C18:1 C18:1). Accordingly, the amount of PC8 is the amount of Phosphatidylcholine(C18:0 C18:2), or the sum of the amounts of Phosphatidylcholine(C18:0 018:2) and Phosphatidylcholine(C18:1 C18:1).

Preferred Biomarker Combinations

As set forth above, the method of the present invention for differentiating in a subject between heart failure and pulmonary disease comprises the determination of the amounts of at least one metabolite biomarker (selected from the biomarkers shown in column 1 of table 1).

In particular, the amounts of at least three biomarkers are determined in a sample of a subject. Thus, the determination of the amounts of a combination of at least three biomarkers is contemplated. Preferred combinations of at least three biomarkers to be determined in accordance with the present invention are described herein below.

The at least three biomarkers for the differentiation between heart failure and pulmonary disease in a subject are preferably:
i. at least one triacylglyceride, at least one cholesterylester, and at least one phosphatidylcholine;
ii. at least one triacylglyceride, at least one phosphatidylcholine, and at least one sphingomyelin;
iii. at least one triacylglyceride, at least one cholesterylester, and at least one sphingomyelin;
iv. at least one phosphatidylcholine, at least one cholesterylester, and at least one sphingomyelin;
v. Cholesterylester C18:2, SSS and Cer(d17:1/24:0);
vi. at least two sphingomyelins selected from the group consisting of SM2, SM3, SM5, SM18, SM23, SM24, and SM28, and at least one triacylglyceride selected from the group consisting of SOP2, SPP1 or PPO1 or selected from the group consisting of SOP2, SPP1 or PPP;
vii. at least two triacylglycerides selected from the group consisting of OSS2, SOP2, SPP1 and SSP2, and at least one sphingomyelin selected from the group consisting of SM23 and SM24;
viii. SM18, SM24 and SM28;
ix. the biomarkers of panel 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, or 199 of Table 2, or
x. the biomarkers of panel 200, 201, 202, 203, 204, 205, or 206 of Table 2a.

Preferred cholesterylester, triacylglyceride, phosphatidylcholine and sphingomyelin biomarkers to be determined and further preferred combinations of at least three biomarkers are described below (in particular for items i., ii., iii., and iv.).

Preferably, the at least one triacylglyceride biomarker in i., ii., and iii. is selected from the group consisting of SOP2, OSS2, SPP1, SSP2, PPO1 and PPP, the at least one cholesterylester biomarker in i., iii. and iv. is selected from the group consisting of cholesterylester 018:2 and cholesterylester C18:0, the at least one phosphatidylcholine biomarker in i., ii., and iv. is selected from the group consisting of PC4 and PC8, and the at least one sphingomyelin biomarker in ii., iii. and iv. is selected from the group consisting of SM18, SM24, SM23, SM21, SM28, SM5, SM3, SM29 and SM8.

Alternatively, the least one triacylglyceride biomarker in i., ii., and iii. is selected from the group consisting of SOP2, OSS2, SPP1, SSP2, PPO1 and SPP1.

In a preferred embodiment, at least the amounts of the biomarkers of items i., ii., iii., vi, vii., ix. or x. (in particular of items i., ii., iii., vi, vii. or ix.) as set forth above are determined. In particular, the at least one triacylglyceride biomarker in i., ii. and iii. is selected from the group consisting of SOP2, OSS2, SSP2, PPO1 and PPP, and/or (in particular and) the at least one cholesterylester biomarker in i. and iii. is cholesterylester 018:2, and/or (in particular and) the at least one phosphatidylcholine biomarker in i. and ii. is PC4, and/or (in particular and) the at least one sphingomyelin biomarker in ii. and iii. is selected from the group consisting of SM18, SM24, SM23, SM28, SM5, and SM3. Preferably, at least the amounts of the biomarkers of any panel of Table 2 which comprises these biomarkers are determined. More preferably, at least the amounts of the biomarkers of panel 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, or 56 in Table 2 are determined.

The term “at least the amounts”, preferably means that, in principle, further biomarkers could be determined. In an embodiment, the term means “the amounts”. Preferably, the amounts of the at least three biomarkers as referred to in step a) of the method disclosed herein, are used for the comparison in step b).

In an even further preferred embodiment, at least the amounts of the biomarkers of i., ii., iii., vi., ix. or x. (in particular of items i., ii., iii., vi. or ix.) are determined, wherein the at least one triacylglyceride biomarker in i., ii. and iii. is SOP2 and/or OSS2, and/or (in particular and) the at least one cholesterylester biomarker in i. and iii. is cholesterylester C18:2, and/or (in particular and) the at least one phosphatidylcholine biomarker in i. and ii. is PC4, and/or (in particular and) the at least one sphingomyelin biomarker in ii. and iii. is selected from the group consisting of SM18, SM24, SM23, and SM3. Preferably, at least the amounts of the biomarkers of any panel of Table 2 which comprises these biomarkers are determined. In particular, at least the amounts of the biomarkers of panel 1, 2, 3, 4, 5, 6, 7, 8, 9, 10, 11, 12, 13, 14, 15, 16, 17, or 18 in Table 2 are determined.

In an even further preferred embodiment, at least the amounts of the biomarkers of i., ii., iii., vi., ix. or x. (in particular of of i., ii., iii, vi, or ix.) are determined, wherein the at least one triacylglyceride biomarker in i., ii. and iii. is SOP2 and/or OSS2, and/or (in particular and) the at least one cholesterylester biomarker in i. and iii. is cholesterylester C18:2, and/or (in particular and) the at least one phosphatidylcholine biomarker in i. and ii. is PC4, and/or (in particular and) the at least one sphingomyelin biomarker in ii. and iii. is SM23. Preferably, at least the amounts of the biomarkers of any panel of Table 2 which comprises these biomarkers are determined. In particular, at least the amounts of the biomarkers of panel 1, 2, 3, or 4 in Table 2 are determined. Preferably, at least the amounts of OSS2, PC4 and SM23 are determined (panel 1), in particular in combination with the determination of the amount of NT-proBNP or BNP (see elsewhere herein). Alternatively, the biomarkers of panels 3, 13 and 60 may be determined. Also preferably, at least the amounts of OSS2, cholesterylester C18:2 and SM23 are determined (panel 2). Alternatively, the biomarkers of panels 20, 21, 22, 23, 32 or 60 may be determined.

In an even further preferred embodiment, at least the amounts of the biomarkers of iii. are determined, wherein the at least one triacylglyceride biomarker is SOP2 and/or OSS2, and/or (in particular and) the at least one cholesterylester biomarker is cholesterylester C18:2, and/or (in particular and) the at least one sphingomyelin biomarker is SM23. In particular, at least the amounts of SOP2, OSS2, PC4, Cholesterylester C18:2, SM18, SM28, SM24, SSP2, and SM23 are determined (panel 3).

As set forth in the previous paragraph, it is, in particular, envisaged to determine the amounts (or to determine at least the amounts of) the biomarkers comprised by panel 3, and thus of SOP2, OSS2, PC4, Cholesterylester C18:2, SM18, SM28, SM24, SSP2, and SM23.

Also preferably, the amounts (or at least the amounts) of at least three biomarkers, of at least four biomarkers, more preferably of at least five or six biomarkers, even more preferably of at least seven biomarkers and most preferably of at least eight biomarkers of the biomarkers of panel 3 are determined in step a) of the method of the present invention.

If the amounts of the biomarkers of panel 3 (or of the biomarkers as set forth in the previous paragraph) are determined, the method preferably does not comprise the further determination of amount of NT-proBNP and/or BNP. In particular, the method may not comprise the further determination of the amount of BNP and/or NT-proBNP and the comparison of the amount of BNP and/or NT-proBNP to a reference. Thus, the differentiation according to the method of the present invention is not based on the determination of NT-proBNP. Accordingly, the method is a non-BNP and non-NT-proBNP based method. The same, e.g., may also apply to panels 1 and 2.

Alternatively, the method may comprise the further determination of amount of NT-proBNP and/or BNP. In particular, the method may comprise the further determination of the amount of BNP or NT-proBNP and the comparison of the amount of BNP or NT-proBNP to a reference.

If the amounts of the biomarkers of panel 3 (of at least three, four, five, six, seven or eight biomarkers of the biomarkers of panel 3) are determined, preferably a correction for confounders is not carried out. Alternatively, a correction for confounders may be carried out. The same may, e.g., apply to panels 1 and 2.

In a preferred embodiment, at least the amounts of the biomarkers shown in i., ii., iii. or vii. are determined, wherein the at least one triacylglyceride biomarker in i., ii. and iii. s selected from the group consisting of SOP2, OSS2, and SSP2, and/or (in particular and) the at least one cholesterylester biomarker in i. and iii. is cholesterylester 018:2, and/or (in particular and) the at least one phosphatidylcholine biomarker in i. and ii. is PC4, and/or (in particular and) the at least one sphingomyelin biomarker in ii. and iii. is selected from the group consisting of SM24, SM23, SM28, and SM3. Preferably, at least the amounts of the biomarkers of any panel of Table 2 which comprises these biomarkers are determined. More preferably, at least the amounts of the biomarkers of panel 19, 20, 21, 22, 23, 24, 25, 26, 27, 28, 29, 30, 31, 32, 33, 34, 35, or 36 in Table 2 are determined.

In an even further preferred embodiment, at least the amounts of the biomarkers shown in ii., or iii. are determined, wherein the at least one triacylglyceride biomarker in ii. and iii. is SOP2 and/or OSS2, and/or (in particular and) the at least one cholesterylester biomarker in iii. is cholesterylester C18:2, and/or (in particular and) the at least one phosphatidylcholine biomarker in ii. is PC4, and/or (in particular and) the at least one sphingomyelin biomarker in ii. and iii. is SM23 and/or SM24. Preferably, at least the amounts of the biomarkers of any panel of Table 2 which comprises these biomarkers are determined. More preferably, at least the amounts of the biomarkers of panel 31, 32, 33, 34, 35, or 36 in Table 2 are determined.

In a preferred embodiment, at least the amounts of the biomarkers shown ii., iii. or vi. are determined, wherein the at least one triacylglyceride biomarker in ii. and iii. is SOP2 and/or OSS2, and/or (in particular and) the at least one cholesterylester biomarker in iii. is cholesterylester C18:2, and/or (in particular and) the at least one phosphatidylcholine biomarker in ii. is PC4, and/or (in particular and) the at least one sphingomyelin biomarker in ii. is selected from the group consisting of SM18, SM24, and SM23. Preferably, at least the amounts of the biomarkers of any panel of Table 2 which comprises these biomarkers are determined. More preferably, at least the amounts of the biomarkers of panel 37, 38, 39, 40, 41, 42, 43, 44, 45, 46, 47, 48, 49, 50, 51, 52, 53, 54, 55, or 56 in Table 2 are determined.

In a further preferred embodiment, at least the amounts of the biomarkers shown in iii. are determined, wherein the at least one triacylglyceride biomarker is SOP2, and/or (in particular and) the at least one cholesterylester biomarker is cholesterylester C18:2, and/or (in particular and) the at least one sphingomyelin biomarker is SM18 and/or SM23. Preferably, at least the amounts of the biomarkers of any panel of Table 2 which comprises these biomarkers are determined. More preferably, at least the amounts of the biomarkers of panel 51, 52, 53, 54, 55, or 56 in Table 2 are determined.

In a preferred embodiment, at least the amounts of the biomarkers of ii., iii., vi., vii., ix. or x. (in particular of ii., iii., vi., vii. or ix.) are determined, and wherein the at least one triacylglyceride biomarker in ii. and iii. is selected from the group consisting of SOP2, OSS2, and PPO1, and/or (in particular and) the at least one cholesterylester biomarker in iii. is cholesterylester C18:2, and/or (in particular and) the at least one phosphatidylcholine biomarker in ii. is PC4, and/or (in particular and) the at least one sphingomyelin biomarker in ii. and iii. is selected from the group consisting of SM24 and SM23. Preferably, at least the amounts of the biomarkers of any panel of Table 2 which comprises these biomarkers are determined. More preferably, at least the amounts of the biomarkers of panel 7, 8, 9, 10, 11, 12, 19, 20, 21, 22, 23, 24, 37, 38, 39, 40, 41, or 42 in Table 2 are determined.

In a further preferred embodiment, wherein at least the amounts of the biomarkers of iii. or vi are determined, wherein the at least one triacylglyceride biomarker in iii. is SOP2, and/or (in particular and) the at least one cholesterylester biomarker in iii. is cholesterylester C18:2, and/or (in particular and) the at least one sphingomyelin biomarker in iii. is selected from the group consisting of SM24 and SM23. Preferably, at least the amounts of the biomarkers of any panel of Table 2 which comprises these biomarkers are determined. More preferably, at least the amounts of the biomarkers of panel 7, 8, 9, 10, 11, or 12 in Table 2 are determined.

In a preferred embodiment, at least the amounts of the biomarkers of iii. or vii. are determined, wherein the at least one triacylglyceride biomarker in iii. is selected from the group consisting of SOP2 and OSS2, and/or (in particular and) the at least one cholesterylester biomarker in iii. is cholesterylester C18:2, and/or (in particular and) the at least one sphingomyelin biomarker in iii. is SM23. Preferably, at least the amounts of the biomarkers of any panel of Table 2 which comprises these biomarkers are determined. More preferably, at least the amounts of the biomarkers of panel 19, 20, 21, 22, 23, or 24 in Table 2 are determined.

In a preferred embodiment, at least the amounts of the biomarkers of iii. or vi. are determined, and wherein the at least one triacylglyceride biomarker in iii. is SOP2, and/or (in particular and) the at least one cholesterylester biomarker in iii. is cholesterylester C18:2, and/or (in particular and) the at least one sphingomyelin biomarker in iii. is selected from the group consisting of SM24 and SM23. Preferably, at least the amounts of the biomarkers of any panel of Table 2 which comprises these biomarkers are determined. More preferably, at least the amounts of the biomarkers of panel 37, 38, 39, 40, 41, or 42 in Table 2 are determined.

In an embodiment, the at least the amounts of the biomarkers of ii., vi. or vii. as set forth above are determined, and wherein the at least one triacylglyceride biomarker in ii. is selected from the group consisting of SOP2, SSP2, SPP1 and PPO1, and/or (in particular and) the at least one phosphatidylcholine biomarker in ii. is selected from the group consisting of PC4 and PC8, and/or (in particular and) the at least one sphingomyelin biomarker in ii. is selected from the group consisting of SM18, SM24, SM23, SM28, SMS, and SM3. Preferably, at least the amounts of the biomarkers of any panel of Table 2 which comprises these biomarkers are determined. More preferably at least the amounts of the biomarkers of panel 79, 80, 81, 82, 83, 84, 85, 86, 87, 88, 89, 90, 91, 92, 93, 94, 95, 96, 97, 98, 99, 100, 101, or 102 in Table 2 are determined. Even more preferably, at least the amounts of the biomarkers of ii. are determined, and wherein the at least one triacylglyceride biomarker is selected from the group consisting of SSP2, SPP1 and PPO1, and/or (in particular and) the at least one phosphatidylcholine biomarker is PC4, and/or (in particular and) the at least one sphingomyelin biomarker is selected from the group consisting of SM24, SMS, and SM3. Most preferably, at least the amounts of the biomarkers of ii. of are determined, and wherein the at least one triacylglyceride biomarker is SSP2, and/or (in particular and) the at least one phosphatidylcholine biomarker is PC4, and/or (in particular and) the at least one sphingomyelin biomarker is selected from the group consisting of SM24 and SMS. Preferably, at least the amounts of the biomarkers of panel 95, 97, 99, or 101 in Table 2 are determined.

In one embodiment, the amount of NT-proBNP or BNP is determined in addition to the amount of the at least one biomarker (selected from the biomarkers shown in column 1 of table 1), in particular in addition to the amount of the resulting combinations of the at least three biomarkers set forth above. In another embodiment, the amount of NT-proBNP or BNP is not determined in addition to the amount of the at least one biomarker or the amounts of the resulting combinations of the at least three biomarkers set forth above (see elsewhere herein).

As set forth above, it is envisaged to determine the amounts of the biomarkers of any one of the panels 1 to 206. The biomarkers of panels 1 to 199 are shown in table 2 (second column). The biomarkers of panels 200 to 206 are shown in table 2a (second column). Preferred panels are panel 1, panel 2, panel 3, panel 4 and panel 200.

In a preferred embodiment, the amounts of the biomarkers of panel 1 or 2 are determined in step a) of the method of the present invention. In another preferred embodiment the biomarkers of panel 200 are determined.

For example, if the amounts of the biomarkers of panel 1 are determined, the amounts of SM23, OSS2 and PC4 are determined. As set forth elsewhere herein, SM23 can be i) Sphingomyelin(d18:1/23:1), ii) Sphingomyelin(d18:2/23:0), iii) Sphingomyelin(d17:1/24:1), or iv) a combination of Sphingomyelin(d18:1/23:1), Sphingomyelin(d18:2/23:0) and Sphingomyelin(d17:1/24:1). In an embodiment, SM23 is Sphingomyelin (d17:1/24:1). In another embodiment, SM23 is Sphingomyelin(d18:1/23:1).

If the biomarkers of panel 1 are determined, it is in particular envisaged to determine:
the amount of OSS2, the amount of Sphingomyelin(d18:1/23:1) and the amount of PC4,
the amount of OSS2, the amount of Sphingomyelin (d17:1/24:1) and the amount of PC4, or
the amount of OSS2, the combined amount of Sphingomyelin(d18:1/23:1), Sphingomyelin(d18:2/23:0), and Sphingomyelin(d17:1/24:1), and the amount of PC4

For example, if the amounts of the biomarkers of panel 2 or 4 are determined, the amounts of SM23, OSS2 and Cholesterylester C18:2 are determined. As set forth elsewhere herein, SM23 can be i) Sphingomyelin(d18:1/23:1), ii) Sphingomyelin(d18:2/23:0), iii) Sphingomyelin(d17:1/24:1), or iv) a combination of Sphingomyelin(d18:1/23:1), Sphingomyelin(d18:2/23:0) and Sphingomyelin(d17:1/24:1). In particular, SM23 is Sphingomyelin (d17:1/24:1) or Sphingomyelin(d18:1/23:1).

If the biomarkers of panel 2 or 4 are determined, it is in particular envisaged to determine:
the amount of OSS2, the amount of Sphingomyelin(d18:1/23:1) and the amount of Cholesterylester C18:2,
the amount of OSS2, the amount of Sphingomyelin (d17:1/24:1) and the amount of Cholesterylester C18:2, or
the amount of OSS2, the combined amount of Sphingomyelin(d18:1/23:1), Sphingomyelin(d18:2/23:0), Sphingomyelin(d17:1/24:1) and the amount of Cholesterylester C18:2

For example, if the amounts of the biomarkers of panel 200 are determined, the amounts of Cholesterylester C18:2, SM23, OSS2 and PC4 are determined. As set forth elsewhere herein, SM23 can be i) Sphingomyelin(d18:1/23:1), ii) Sphingomyelin(d18:2/23:0), iii) Sphingomyelin(d17:1/24:1), or iv) a combination of Sphingomyelin(d18:1/23:1), Sphingomyelin(d18:2/23:0) and Sphingomyelin(d17:1/24:1). In an embodiment, SM23 is Sphingomyelin (d17:1124:1). In another embodiment, SM23 is Sphingomyelin(d18:1/23:1).

If the biomarkers of panel 200 are determined, it is in particular envisaged to determine:
the amount of Cholesterylester C18:2, the amount of OSS2, the amount of Sphingomyelin(d18:1/23:1) and the amount of PC4,
the amount of Cholesterylester C18:2, the amount of OSS2, the amount of Sphingomyelin (d17:1/24:1) and the amount of PC4, or
the amount of Cholesterylester C18:2, the amount of OSS2, the combined amount of Sphingomyelin(d18:1/23:1), Sphingomyelin(d18:2/23:0), and Sphingomyelin(d17:1/24:1), and the amount of PC4

In addition to panel 1, panel 200 further comprises Cholesterylester C18:2.

In addition to panels 2 and 4, panel 200 further comprises PC4.

As set forth elsewhere herein, it is further envisaged to determine the amount of NT-proBNP or BNP in addition to the amount of the at least one biomarker selected from the biomarkers shown in column 1 of Table 1 or in addition to the amounts of the biomarkers of the aforementioned panels (1 to 206). For example, NT-proBNP or BNP is determined in addition to the amounts of the at least three biomarkers of panel 1. Moreover, in an embodiment, it is envisaged that no correction for confounders is carried out (e.g. for panel 1).

Further preferred combinations of at least three biomarkers for the differentiation between heart failure and pulmonary disease are disclosed in the following:

Moreover, the at least three biomarkers, preferably are as follows. In particular it is envisaged to determine in step a. the amounts of
i. at least one sphingomyelin (SM) biomarker selected from the group consisting of SM18, SM21, SM23, SM24, SM28, SM3, SMS, SM2, SM9 and SM10, in particular at least one sphingomyelin (SM) biomarker selected from the group consisting of SM18, SM21, SM23, SM24, SM28, SM3 and SMS,
ii. at least one triacylglyceride biomarker selected from the group consisting of OSS2, PPO1, SOP2, SSP2 and SPP1, in particular at least one triacylglyceride biomarker selected from the group consisting of OSS2, PPO1, SOP2, and SSP2, and
iii. at least one further biomarker selected from the group consisting of Cer(d16:1/24:0), Cer(d18:1/24:1), Cholesterylester C18:2, PC4, PC8, Cer(d18:2/24:0), Cer(d17:1/24:0) and glutamic acid 1, in particular at least one further biomarker selected from the group consisting of Cer(d16:1/24:0), Cer(d18:1/24:1), Cholesterylester C18:2, and PC4.

Thus, at least one biomarker of i., at least one biomarker of ii., and at least one biomarker of iii. are determined. The same applies to the combinations below.

In a preferred embodiment, at least the amounts of
i. at least one sphingomyelin biomarker selected from the group consisting of SM28, SM23, SM21, and SM5,
ii. SOP2 and/or OSS2, and
iii. Cholesterylester C18:2 and/or PC4 are determined.

In a further preferred embodiment, at least the amounts of
i. at least one sphingomyelin biomarker selected from the group consisting of SM28, SM21, and SM5, in particular SM28, or at least one sphingomyelin biomarker selected from the group consisting of SM18, SM21, and SM5, in particular SM18, and
ii. SOP2, and
iii. PC4

are determined.

In a further preferred embodiment, wherein at least the amounts of
i. at least one sphingomyelin biomarker selected from the group consisting of SM18, SM2, SM23, SM24, SM28, SM3, and SM5,
ii. at least one triacylglyceride biomarker selected from the group consisting of OSS2, PPO1, SOP2, SSP2 and SPP1, and
iii. at least one further biomarker selected from the group consisting of Cer(d16:1/24:0), Cholesterylester C18:2, PC4 and glutamic acid 1

are determined.

In a further preferred embodiment, at least the amounts of
i. at least one sphingomyelin biomarker selected from the group consisting of SM23, SM2, and SM24,
ii. at least one triacylglyceride biomarker selected from the group consisting of SOP2, OSS2, and PPO1, and
iii. Cholesterylester C18:2 and/or PC4, in particular Cholesterylester C18:2, are determined.

In particular, at least the amounts of SM23 and/or SM2, in particular of SM23, of SOP2, and of PC4 are determined.

In a preferred embodiment of the present invention, at least the amounts of
i. at least one sphingomyelin biomarker selected from the group consisting of SM10, SM18, SM21, SM23, SM24, SM28, SM3 and SM9,
ii. at least one triacylglyceride biomarker selected from the group consisting of OSS2, PPO1, SOP2, SSP2 and SPP1, and
iii. at least one further biomarker selected from the group consisting of Cer(d16:1/24:0), Cer(d18:1/24:1), Cer(d17:1/24:0), Cer (d18:2/24:0), Cholesterylester C18:2, PC4 and PC8

are determined.

In further preferred embodiment
i. at least one sphingomyelin biomarker is selected from the group consisting of SM18, SM3, SM24, and SM23, in particular SM18,
ii. SOP2 and/or OSS2, in particular SOP2, and
iii. Cholesterylester C18:2 and/or PC4, in particular PC4, are determined.

In particular, least the amounts of SM18 and/or SM24, SOP2, and PC4 are determined.

In a preferred embodiment, wherein at least the amounts of
i. at least one sphingomyelin biomarker selected from the group consisting of SM23, SM18, SM24, SM28, SM2 and SM3, in particular at least one sphingomyelin biomarker selected from the group consisting of SM23, SM18, SM24, and SM28,
ii. OSS2 and/or SOP2, in particular OSS2,
iii. Cholesterylester C18:2 and/or PC4

are determined.

In particular, at least the amounts of SM23, OSS2, and Cholesterylester C18:2 are determined. In a further preferred embodiment, at least the amounts of
i. at least one sphingomyelin biomarker selected from the group consisting of SM23 SM18, SM2, SM24 and SM28,
ii. SOP2, and
iii. Cholesterylester C18:2

are determined.

In particular, at least the amounts of biomarkers SM23, SOP2, and Cholesterylester C18:2 are determined.

In a preferred embodiment, wherein at least the amounts of
i. at least one sphingomyelin biomarker selected from the group consisting of SM18, SM23, SM3 or at least one sphingomyelin biomarker selected from the group consisting of SM28, SM23 and SM3,
ii. OSS2, and
iii. Cholesterylester C18:2 and/or PC4

are determined.

In particular, at least the amounts of SM18 and/or SM28, of OSS2, and of Cholesterylester C18:2 are determined.

The aforementioned combinations are preferably used for the diagnosis of heart failure, in particular of HFrEF. In an embodiment, the heart failure is symptomatic heart failure, in particular symptomatic HFrEF. Accordingly, the subject to be tested preferably shows symptoms of heart failure.

In a preferred embodiment of the present invention, at least the amounts of
i. at least one sphingomyelin biomarker selected from the group consisting of SM23, SM24, SM28, and SM3, in particular at least one sphingomyelin biomarker selected from the group consisting of SM23, SM24, and SM28,
ii. SOP2 and/or OSS2, and
iii. Cholesterylester C18:2 and/or PC4

are determined.

In particular, at least the amounts of SM23, of SOP2, and of Cholesterylester C18:2 are determined.

In a further preferred embodiment of the present invention, at least the amounts of
i. SM23,
ii. OSS2 and/or SOP2, and
iii. Cholesterylester C18:2
are determined.

In particular, at least the amounts of SM23, OSS2, and Cholesterylester 018:2 are determined.

In a preferred embodiment of the present invention at least the amounts of
i. SM28 and/or SM3,
ii. SOP2 and/or OSS2, and
iii. PC4

are determined.

In particular, at least the amounts of SM28, SOP2, and of PC4 are determined.

In another preferred embodiment, wherein at least the amounts of
i. at least one sphingomyelin biomarker selected from the group consisting of SM23, SM24, SM28 and SM18, in particular at least one sphingomyelin biomarker selected from the group consisting of SM23, SM24, and SM28,
ii. SOP2, and
iii. Cholesterylester C18:2

are determined.

In particular, at least the amounts of SM23, SOP2, and Cholesterylester C18:2 are determined.

In a further preferred embodiment, at least the amounts of SM18, SOP2, and Cholesterylester C18:2 are determined.

It is further envisaged that the at least three are biomarkers are:
i. at least one sphingomyelin biomarker selected from the group consisting of SM23, SM2, and SM24,
ii. at least one triacylglyceride biomarker selected from the group consisting of SOP2, OSS2, and PPO1, and
iii. at least one further biomarker selected from Cholesterylester C18:2, PC4, and SM5.

Thus, the subject preferably does not show symptoms of heart failure.

It is further envisaged that the at least three biomarkers are markers are selected from the group consisting of SM24, SM5, SM23, SOP2 and PPO1. Preferably, the amounts of SM24, SM5 and SOP2, or of SM23, SM5 and PPO1 are determined.

In one embodiment, the amount of NT-proBNP or BNP is determined in addition to the amount of the resulting combinations of the at least three biomarkers set forth above. In another embodiment, the amount of NT-proBNP or BNP is not determined in addition to the amount of the resulting combinations of the at least three biomarkers set forth above (see elsewhere herein).

Additional preferred combinations of at least three biomarkers for the diagnosis of heart failure and/or subforms thereof

In a preferred embodiment, the least three biomarkers are:
i. at least one sphingomyelin (SM) biomarker selected from the group consisting of SM18, SM23, SM24, SM3, SM5, SM2, and SM28, and in particular at least one sphingomyelin (SM) biomarker selected from the group consisting of SM18, SM23, SM24, SM3, and SM5,
ii. at least one triacylglyceride biomarker selected from the group consisting of OSS2, SOP2, SSP2 and PPO1, in particular at least one triacylglyceride biomarker selected from the group consisting of OSS2 and SOP2, and
iii. at least one further biomarker selected from the group consisting of Cholesterylester 018:2 and PC4.

Thus, at least one biomarker of i., at least one biomarker of ii., and at least one biomarker of iii. are determined. The same applies to the combinations below.

In a preferred embodiment, at least the amounts of
i. at least one sphingomyelin biomarker selected from the group consisting of SM18, SM23, SM24, SM3, and SM5,
ii. at least one triacylglyceride biomarker selected from the group consisting of OSS2 and SOP2, and
iii. at least one further biomarker selected from the group consisting of Cholesterylester 018:2 and PC4

are determined.

In particular, at the least the amounts of
i. at least one sphingomyelin biomarker selected from the group consisting of SM18, SM3, and SM5, in particular SM18, and
ii. at least one triacylglyceride biomarker selected from the group consisting of OSS2 and SOP2, in particular SOP2, and
iii. at least one further biomarker selected from the group consisting of Cholesterylester 018:2 and PC4, in particular PC4

are determined.

In a preferred embodiment, at least the amounts of
i. at least one sphingomyelin biomarker selected from the group consisting of SM18, SM2, SM23, SM24, SM28, and SM5,
ii. at least one triacylglyceride biomarker selected from the group consisting of OSS2, PPO1, SOP2, and SSP2, and
iii. at least one further biomarker selected from the group consisting of Cholesterylester C18:2 and PC4

are determined.

In a more preferred embodiment, at least the amounts of
i. at least one sphingomyelin biomarker selected from the group consisting of SM23, SM24, SM18, and SM2, in particular SM23,
ii. at least one triacylglyceride biomarker selected from the group consisting of SOP2, OSS2, and PPO1, in particular SOP2, and
iii. at least one further biomarker selected from the group consisting of Cholesterylester C18:2 and PC4, in particular Cholesterylester C18:2, are determined.

In an even more preferred embodiment, at least the amounts of
i. at least one sphingomyelin biomarker selected from the group consisting of SM23, SM18, and SM2, in particular SM23,
ii. SOP2, and
iii. Cholesterylester C18:2 and/or PC4, in particular Cholesterylester C18:2,

are determined.

In a preferred embodiment,
i. at least one sphingomyelin biomarker selected from the group consisting of SM18, SM23, SM24, SM28, and SM3,
ii. at least one triacylglyceride biomarker selected from the group consisting of OSS2 and SOP2, and
iii. at least one further biomarker selected from the group consisting of Cholesterylester C18:2 and PC4

are determined.

Preferably, at least the amounts of
i. SM18,
ii. SOP2, and
iii. Cholesterylester C18:2,

are determined.

In particular, at least the amounts of SM18 and/or SM24, in particular SM18, of SOP2, and of PC4 are determined.

In a further preferred embodiment, at least the amounts of
i. at least one sphingomyelin biomarker selected from the group consisting of SM23, SM18, SM3, SM24, SM 28 in particular at least one sphingomyelin biomarker selected from the group consisting of SM23 and SM18,
ii. at least one triacylglyceride biomarker selected from the group consisting of OSS2, SOP2 and PPO1, in particular OSS2, and
iii. at least one further biomarker selected from the group consisting of Cholesterylester 018:2 and PC4

are determined.

In particular, at least the amounts of SM23, OSS2, and Cholesterylester C18:2 are determined.

In a further preferred embodiment, at least the amounts of
i. at least one sphingomyelin biomarker selected from the group consisting of SM23 and SM24, in particular SM23,
ii. at least one triacylglyceride biomarker selected from the group consisting of SOP2, and OSS2, in particular SOP2, and
iii. at least one further biomarker selected from the group consisting of Cholesterylester 018:2 and PC4, in particular Cholesterylester C18:2

are determined.

In particular, at least the amounts of SM23, SOP2, and Cholesterylester C18:2 are determined. In a further preferred embodiment, at least the amounts of
i. at least one sphingomyelin biomarker selected from the group consisting of SM18, SM23, SM3, SM28 and SM24, in particular at least one sphingomyelin biomarker selected from the group consisting of SM18, SM23 and SM3,
ii. at least one triacylglyceride biomarker selected from the group consisting of SOP2 and OSS2, and
iii. at least one further biomarker selected from the group consisting of Cholesterylester 018:2 and PC4

are determined.

In particular, at least the amounts of SM18, of OSS2, and of Cholesterylester C18:2 are determined.

In a further preferred embodiment, at least the amounts of
i. at least one sphingomyelin biomarker selected from the group consisting of SM23, SM24, SM3 and SM28, in particular at least one sphingomyelin biomarker selected from the group consisting of SM23 and SM24,
ii. at least one triacylglyceride biomarker selected from the group consisting of SOP2, and OSS2, and
iii. at least one further biomarker selected from the group consisting of Cholesterylester 018:2 and PC4

are determined.

In particular, at least the amounts of SM23, of SOP2, and of Cholesterylester C18:2 are determined.

In a further preferred embodiment, wherein at least the amounts of
i. at least one sphingomyelin biomarker selected from the group consisting of SM23 and SM24,
ii. at least one triacylglyceride biomarker selected from the group consisting of SOP2 and OSS2, and
iii. Cholesterylester C18:2

are determined.

In particular, at least the amounts of SM23, OSS2, and Cholesterylester 018:2 are determined.

In a further preferred embodiment, at least the amounts of
i. at least one sphingomyelin biomarker selected from the group consisting of SM3, SM24 and SM28,
ii. at least one triacylglyceride biomarker selected from the group consisting of SOP2 and OSS2, and
iii. at least one further biomarker selected from the group consisting of Cholesterylester 018:2 and PC4

are determined.

In particular, at least the amounts of SM3, OSS2, and of PC4 are determined.

In a further preferred embodiment, at least the amounts of
i. at least one sphingomyelin biomarker selected from the group consisting of SM18, SM23, and SM24, in particular at least one sphingomyelin biomarker selected from the group consisting of SM18 and SM23,
ii. at least one triacylglyceride biomarker selected from the group consisting of SOP2 and PPO1, in particular SOP2, and
iii. at least one further biomarker selected from the group consisting of Cholesterylester C18:2 and PC4, in particular Cholesterylester C18:2

are determined.

In particular, at least the amounts of SM18, SOP2, and Cholesterylester C18:2 are determined. In a further preferred embodiment, at least the amounts of
i. at least one sphingomyelin biomarker selected from the group consisting of SM24 and SM23,
ii. at least one triacylglyceride biomarker selected from the group consisting of SOP2 and PPO1, and
iii. at least one further biomarker selected from the group consisting of Cholesterylester C18:2 and PC4, in particular Cholesterylester C18:2

are determined.

In particular, at least the amounts of SM24, SOP2, and Cholesterylester C18:2 are determined.

In an even further preferred embodiment, at least the amounts of SM18, SOP2, and Cholesterylester 018:2 are determined.

Moreover, for the diagnosis of asymptomatic heart failure it is envisaged to determine the amounts of
i. at least one sphingomyelin biomarker selected from the group consisting of SM23, SM24, SM18, and SM2, in particular SM23,
ii. at least one triacylglyceride biomarker selected from the group consisting of SOP2, OSS2, and PPO1, in particular SOP2, and
iii. at least one further biomarker selected from the group consisting of Cholesterylester C18:2, PC4, SM5 and SSP2, in particular Cholesterylester 018:2.

Further, it is envisaged to determine the amounts of
i. at least one sphingomyelin biomarker selected from the group consisting of SM23, SM18, and SM2, in particular SM23,
ii. SOP2, and
iii. at least one further biomarker selected from the group consisting of SM5, Cholesterylester C18:2 PC4, in particular SM5.

Further, it is envisaged to determine the amounts of
i. at least one sphingomyelin biomarker selected from the group consisting of SM23 and SM24,
ii. at least one triacylglyceride biomarker selected from the group consisting of SOP2, and OSS2, in particular SOP2, and
iii. at least one further biomarker selected from the group consisting of Cholesterylester C18:2 and SM28, in particular Cholesterylester C18:2.

Further, it is is envisaged to determine the amounts of
i. at least one sphingomyelin biomarker selected from the group consisting of SM23 and SM24, in particular SM23,
ii. at least one triacylglyceride biomarker selected from the group consisting of SOP2 and OSS2, in particular OSS2 and
iii. Cholesterylester C18:2 and SSP2, in particular Cholesterylester 018:2.

Further, it is envisaged to determine the amounts of
i. at least one sphingomyelin biomarker selected from the group consisting of SM24 and SM23, in particular SM24,
ii. at least one triacylglyceride biomarker selected from the group consisting of SOP2 and PPO1, in particular SOP2, and
iii. at least one further biomarker selected from the group consisting of SMS, Cholesterylester C18:2 and PC4, in particular Cholesterylester C18:2, in particular SMS, or in particular PC4.

In one embodiment, the amount of NT-proBNP or BNP is determined in addition to the amount of the resulting combinations of the at least three biomarkers set forth above. In another embodiment, the amount of NT-proBNP or BNP is not determined in addition to the amount of the resulting combinations of the at least three biomarkers set forth above (see elsewhere herein).

If the one or more biomarker(s) to be determined is a triacylglyceride it is envisaged that the determination of the amount of this biomarker does not encompass the derivatization of this marker. Accordingly, it is envisaged that the determination of this biomarker is not based on the determination of one or more of the fatty acid residues derived from said triacylglyceride. Accordingly, the amount of the entire triacylglyceride is measured. Nevertheless, a derivatization can be carried out.

The term “sample” as used herein refers to samples from body fluids, preferably, blood, plasma, serum, saliva or urine, or samples derived, e.g., by biopsy, from cells, tissues or organs, in particular from the heart. More preferably, the sample is a blood, plasma or serum sample, most preferably, a plasma sample. Biological samples can be derived from a subject as specified elsewhere herein. Techniques for obtaining the aforementioned different types of biological samples are well known in the art. For example, blood samples may be obtained by blood taking while tissue or organ samples are to be obtained, e.g., by biopsy.

In a preferred embodiment of the present invention, the sample is fasting sample, in particular a fasting blood, serum or plasma sample. Accordingly, the sample shall have been obtained from a fasting subject. A fasting subject, in particular, is a subject who refrained from food and beverages, except for water, prior to obtaining the sample to be tested. Preferably, a fasting subject refrained from food and beverages, except for water, for at least eight hours prior to obtaining the sample to be tested. More preferably, the sample has been obtained from the subject after an overnight fast.

The aforementioned samples are, preferably, pre-treated before they are used for the method of the present invention. As described in more detail below, said pre-treatment may include treatments required to release or separate the compounds or to remove excessive material or waste. Suitable techniques comprise centrifugation, extraction, fractioning, ultrafiltration, protein precipitation followed by filtration and purification and/or enrichment of compounds. Moreover, other pre-treatments are carried out in order to provide the compounds in a form or concentration suitable for compound analysis. For example, if gas-chromatography coupled mass spectrometry is used in the method of the present invention, it will be required to derivatize the compounds prior to the said gas chromatography. Suitable and necessary pre-treatments depend on the means used for carrying out the method of the invention and are well known to the person skilled in the art. Pre-treated samples as described before are also comprised by the term “sample” as used in accordance with the present invention.

As set forth herein below in more detail, the determination of the amount of a biomarker as referred to herein, preferably includes a separation step, i.e. a step in which compounds comprised by the sample are separated. Preferably, the separation of the compounds is carried by chromatography, in particular by liquid chromatography (LC) or high performance liquid chromatography (HPLC). Preferably, the pre-treatment of the sample should allow for a subsequent separation of compounds, in particular the metabolite biomarkers as referred to above, comprised by the sample. Molecules of interest, in particular the biomarkers as referred to above may be extracted in an extraction step which comprises mixing of the sample with a suitable extraction solvent. The extraction solvent shall be capable of precipitating the proteins in a sample, thereby facilitating the, preferably, centrifugation-based, removal of protein contaminants which otherwise would interfere with the subsequent analysis of the biomarkers as referred above. Preferably, the metabolite biomarkers to be determined, in particular all lipid biomarkers as referred above, are soluble in the extraction solvent. More preferably, the extraction solvent is a one phase solvent. Even more preferably, the extraction solvent is a mixture comprising a first solvent selected from the group consisting of dichloromethane (DCM), chloroform, tertiary butyl methyl ether (tBME or MTBE, also known as 2-methoxy-2-methylpropane), ethyl ethanoate, and isooctane, and a second solvent selected from the group consisting of methanol, ethanol, isopropanol and dimethyl sulfoxide (DMSO). In an embodiment the extraction solvent comprises methanol and DCM, in particular a ratio of about 2:1 to about 3:2, preferably a ratio of about 2:1 or about 3:2 (preferably v/v). The term “about” as used herein refers to either the precise value indicated afterwards or to a value differing +/−20%, +/−10%, +/−5%, +/−2% or +/−1% from the said precise value.

Preferably, the pretreatment of the sample comprises an extraction step with a suitable extraction solvent. This extraction step additionally results in the precipitation of proteins comprised by the sample. Subsequently, the proteins comprised by the sample are removed by centrifugation.

As described herein below, the method of the present invention may further comprise the determination of the amount of a natriuretic peptide, preferably NT-proBNP or BNP. Natriuretric peptides are protein markers which are preferably determined by using antibodies which specifically bind to BNP or NT-proBNP (or alternatively by other methods known in the art). The pretreatment as described in the two paragraphs above, thus, does not apply to the determination of protein markers, in particular BNP and NT-proBNP. Preferably, the pre-treatment applies to the determination of the amount of the metabolite biomarkers only, i.e. of sphingomyelins, triacylglycerides, cholesterylesters, phosphatidylcholines, ceramides and/or glutamic acid.

The term “determining the amount”, in particular of the metabolite biomarkers, as used herein refers to determining at least one characteristic feature of a biomarker to be determined by the method of the present invention in the sample. Characteristic features in accordance with the present invention are features which characterize the physical and/or chemical properties including biochemical properties of a biomarker. Such properties include, e.g., molecular weight, viscosity, density, electrical charge, spin, optical activity, colour, fluorescence, chemiluminescence, elementary composition, chemical structure, capability to react with other compounds, capability to elicit a response in a biological read out system (e.g., induction of a reporter gene) and the like. Values for said properties may serve as characteristic features and can be determined by techniques well known in the art. Moreover, the characteristic feature may be any feature which is derived from the values of the physical and/or chemical properties of a biomarker by standard operations, e.g., mathematical calculations such as multiplication, division or logarithmic calculus. Most preferably, the at least one characteristic feature allows the determination and/or chemical identification of the said at least one biomarker and its amount. Accordingly, the characteristic value, preferably, also comprises information relating to the abundance of the biomarker from which the characteristic value is derived. For example, a characteristic value of a biomarker may be a peak in a mass spectrum. Such a peak contains characteristic information of the biomarker, i.e. the m/z information, as well as an intensity value being related to the abundance of the said biomarker (i.e. its amount) in the sample.

As discussed before, each biomarker comprised by a sample may be, preferably, determined in accordance with the present invention quantitatively or semi-quantitatively. For quantitative determination, either the absolute or precise amount of the biomarker will be determined or the relative amount of the biomarker will be determined based on the value determined for the characteristic feature(s) referred to herein above. The relative amount may be determined in a case were the precise amount of a biomarker can or shall not be determined. In said case, it can be determined whether the amount in which the biomarker is present, is enlarged or diminished with respect to a second sample comprising said biomarker in a second amount. In a preferred embodiment said second sample comprising said biomarker shall be a calculated reference as specified elsewhere herein. Quantitatively analysing a biomarker, thus, also includes what is sometimes referred to as semi-quantitative analysis of a biomarker.

Thus, the determination of the amount of a biomarker as referred to herein is preferably done by a compound separation step and a subsequent mass spectrometry step. Thus, determining as used in the method of the present invention, preferably, includes using a compound separation step prior to the analysis step. Preferably, said compound separation step yields a time resolved separation of the metabolites, in particular of the at least one or the at least three biomarkers as set forth in connection with the method of the present invention, comprised by the sample. Suitable techniques for separation to be used preferably in accordance with the present invention, therefore, include all chromatographic separation techniques such as liquid chromatography (LC), high performance liquid chromatography (HPLC), gas chromatography (GC), thin layer chromatography, size exclusion or affinity chromatography. These techniques are well known in the art and can be applied by the person skilled in the art without further ado. Most preferably, LC and/or HPLC are chromatographic techniques to be envisaged by the method of the present invention. Suitable devices for such determination of biomarkers are well known in the art. Preferably, mass spectrometry is used in particular gas chromatography mass spectrometry (GC-MS), liquid chromatography mass spectrometry (LC-MS), direct infusion mass spectrometry or Fourier transform ion-cyclotrone-resonance mass spectrometry (FT-ICR-MS), capillary electrophoresis mass spectrometry (CE-MS), high-performance liquid chromatography coupled mass spectrometry (HPLC-MS), quadrupole mass spectrometry, any sequentially coupled mass spectrometry, such as MS-MS or MS-MS-MS, inductively coupled plasma mass spectrometry (ICP-MS), pyrolysis mass spectrometry (Py-MS), ion mobility mass spectrometry or time of flight mass spectrometry (TOF). More preferably, LC-MS, in particular LC-MS/MS, and most preferably HPLC-MS, in particular HPLC-MS/MS, are used as described in detail below. Accordingly, the determination of the amount(s) to the at least one biomarker or the at least three biomarkers is preferably carried by HPLC-MS, in particular HPLC-MS/MS (high-performance liquid chromatography tandem mass spectrometry). The techniques described above are disclosed in, e.g., Nissen 1995, Journal of Chromatography A, 703: 37-57, U.S. Pat. No. 4,540,884 or U.S. Pat. No. 5,397,894, the disclosure content of which is hereby incorporated by reference.

As an alternative or in addition to mass spectrometry techniques, the following techniques may be used for compound determination: nuclear magnetic resonance (NMR), magnetic resonance imaging (MRI), Fourier transform infrared analysis (FT-IR), ultraviolet (UV) spectroscopy, refraction index (RI), fluorescent detection, radiochemical detection, electrochemical detection, light scattering (LS), dispersive Raman spectroscopy or flame ionisation detection (FID). These techniques are well known to the person skilled in the art and can be applied without further ado.

The method of the present invention shall be, preferably, assisted by automation. For example, sample processing or pre-treatment can be automated by robotics. Data processing and comparison is, preferably, assisted by suitable computer programs and databases. Automation as described herein before allows using the method of the present invention in high-throughput approaches.

As described above, said determining of the amount of the at least one biomarker, in particular of the at least three biomarkers can, preferably, comprise mass spectrometry (MS). Thus, a mass spectrometry step is carried out after the separation step (e.g. by LC or HPLC). Mass spectrometry as used herein encompasses all techniques which allow for the determination of the molecular weight (i.e. the mass) or a mass variable corresponding to a compound, i.e. a biomarker, to be determined in accordance with the present invention. Preferably, mass spectrometry as used herein relates to GC-MS, LC-MS, direct infusion mass spectrometry, FT-ICR-MS, CE-MS, HPLC-MS, quadrupole mass spectrometry, any sequentially coupled mass spectrometry such as MS-MS or MS-MS-MS, ICP-MS, Py-MS, TOF or any combined approaches using the aforementioned techniques. How to apply these techniques is well known to the person skilled in the art. Moreover, suitable devices are commercially available. More preferably, mass spectrometry as used herein relates to LC-MS and/or HPLC-MS, i.e. to mass spectrometry being operatively linked to a prior liquid chromatography separation step. Preferably, the mass spectrometry is tandem mass spectrometry (also known as MS/MS). Tandem mass spectrometry, also known as MS/MS involves two or more mass spectrometry step, with a fragmentation occurring in between the stages. In tandem mass spectrometry two mass spectrometers in a series connected by a collision cell. The mass spectrometers are coupled to the chromatographic device. The sample that has been separated by a chromatography is sorted and weighed in the first mass spectrometer, then fragmented by an inert gas in the collision cell, and a piece or pieces sorted and weighed in the second mass spectrometer. The fragments are sorted and weighed in the second mass spectrometer. Identification by MS/MS is more accurate.

In an embodiment, mass spectrometry as used herein encompasses quadrupole MS. Most preferably, said quadrupole MS is carried out as follows: a) selection of a mass/charge quotient (m/z) of an ion created by ionisation in a first analytical quadrupole of the mass spectrometer, b) fragmentation of the ion selected in step a) by applying an acceleration voltage in an additional subsequent quadrupole which is filled with a collision gas and acts as a collision chamber, c) selection of a mass/charge quotient of an ion created by the fragmentation process in step b) in an additional subsequent quadrupole, whereby steps a) to c) of the method are carried out at least once.

More preferably, said mass spectrometry is liquid chromatography (LC) MS such high performance liquid chromatography (HPLC) MS, in particular HPLC-MS/MS. Liquid chromatography as used herein refers to all techniques which allow for separation of compounds (i.e. metabolites) in liquid or supercritical phase. Liquid chromatography is characterized in that compounds in a mobile phase are passed through the stationary phase. When compounds pass through the stationary phase at different rates they become separated in time since each individual compound has its specific retention time (i.e. the time which is required by the compound to pass through the system). Liquid chromatography as used herein also includes HPLC. Devices for liquid chromatography are commercially available, e.g. from Agilent Technologies, USA. For examples, HPLC can be carried out with commercially available reversed phase separation columns with e.g. C8, C18 or C30 stationary phases. The person skilled in the art is capable to select suitable solvents for the HPLC or any other chromatography method as described herein. The eluate that emerges from the chromatography device shall comprise the biomarkers as referred to above.

A suitable solvent for elution for lipid chromatography can be determined by the skilled person. In an embodiment, the solvents for gradient elution in the HPLC separation consist of a polar solvent and a lipid solvent. Preferably, the polar solvent is a mixture of water and a water miscible solvent with an acid modifier. Examples of suitable organic solvents which are completely miscible with water include the C1-C3-alkanols, tetrahydrofurane, dioxane, C3-C4-ketones such as acetone and acetonitril and mixtures thereof, with methanol being particularly preferred. Additionally the lipid solvent is a mixture of the above mentioned solvents together with hydrophobic solvents from the groups consisting of dichloromethane (DCM), chloroform, tertiary butyl methyl ether (tBME or MTBE), ethyl ethanoate, and isooctane. Examples of acidic modifiers are formic acid or acidic acid. Preferred solvents for gradient elution are disclosed in the Examples section.

Gas chromatography which may be also applied in accordance with the present invention, in principle, operates comparable to liquid chromatography. However, rather than having the compounds (i.e. metabolites) in a liquid mobile phase which is passed through the stationary phase, the compounds will be present in a gaseous volume. The compounds pass the column which may contain solid support materials as stationary phase or the walls of which may serve as or are coated with the stationary phase. Again, each compound has a specific time which is required for passing through the column. Moreover, in the case of gas chromatography it is preferably envisaged that the compounds are derivatized prior to gas chromatography. Suitable techniques for derivatization are well known in the art. Preferably, derivatization in accordance with the present invention relates to methoxymation and trimethylsilylation of, preferably, polar compounds and transmethylation, methoxymation and trimethylsilylation of, preferably, nonpolar (i.e. lipophilic) compounds.

For mass spectrometry, the analytes in the sample are ionized in order to generate charged molecules or molecule fragments. Afterwards, the mass-to-charge of the ionized analyte, in particular of the ionized biomarkers, or fragments thereof is measured.

Thus, the mass spectrometry step preferably comprises an ionization step in which the biomarkers to be determined are ionized. Of course, other compounds present in the sample/elulate are ionizied as well. Ionization of the biomarkers can be carried out by any method deemed appropriate, in particular by electron impact ionization, fast atom bombardment, electrospray ionization (ESI), atmospheric pressure chemical ionization (APCI), matrix assisted laser desorption ionization (MALDI).

Preferably, the ionization step is carried out by atmospheric pressure chemical ionization (AP-CI). More preferably, the ionization step (for mass spectrometry) is carried out by electrospray ionization (ESI). Accordingly, the mass spectrometry is preferably ESI-MS (or if tandem MS is carried out: ESI-MS/MS). Electrospray is a soft ionization method which results in the formation of ions without breaking any chemical bonds.

Electrospray ionization (ESI) is a technique used in mass spectrometry to produce ions using an electrospray in which a high voltage is applied to the sample to create an aerosol. It is especially useful in producing ions from macromolecules because it overcomes the propensity of these molecules to fragment when ionized.

Preferably, the electrospray ionization is positive ion mode electrospray ionization. Thus, the ionization is preferably a protonation (or an adduct formation with positive charged ions such as NH₄⁺, Na⁺, or K⁺, in particular NH₄⁺). According a suitable cation, preferably, a proton (H⁺) is added to the biomarkers to be determined (and of course to any compound in the sample, i.e. in the eluate from the chromatography column). Therefore, the determination of the amount of the at least one biomarker, in particular of the amounts of the at least three biomarkers might be the determination of the amount of protonated biomarkers.

The person skilled in the art knows that the ionization step is carried out at the beginning of the mass spectrometry step. If tandem MS is carried out, the ionization, in particular the electrospray ionization, is carried out in the first mass spectrometry step.

The ionization of the biomarkers can be preferably carried out by feeding the liquid eluting from the chromatography column (in particular from the LC or HPLC column) directly to an electrospray. Alternatively the fractions can be collected and are later analyzed in a classical nanoelectrospray-mass spectrometry setup.

As set forth above, the mass spectrometry step is carried out after the separation step, in particular the chromatography step. In an embodiment, the eluate that emerges from the chrometography column (e.g. the LC or HPLC column) may be pre-treated prior to subjecting it to the mass spectrometry step. Preferably, ammonium buffer (most preferably ammonium formate or ammonium acetate) is added to the eluate in order to enhance the ionization efficiency in the electrospray process for some lipids such as ceramides and TAGs. Preferably, the ammonium formate buffer is dissolved in a solvent miscible with the gradient HPLC solvents (most preferably methanol).

In a preferred embodiment of the present invention, the at least three biomarkers are determined together in a single measurement. In particular, it is envisaged to determine the amounts together in a single LC-MS (or LC-MS/MS), HPLC-MS (HPLC-MS/MS) measurement (i.e. run).

In an embodiment, the determination of the amounts of the at least three biomarkers comprises steps as described in the Examples section. For example, the at least three biomarkers can be determined as described in Example 3. In particular, the biomarkers of panels 1, 2 or 200 can be determined as described in the Examples section (preferably, Example 3).

For example, a blood, serum, or plasma sample (in particular a plasma sample) can be analyzed. The sample may be a fresh sample or a frozen sample. If frozen, the sample may be thawed at suitable temperature for a suitable time. An aliquot of the sample (e.g. said aliquot of the sample my have a volume of 10 μl) is then transferred to microcentrifuge tube and mixed with a suitable extraction solvent (e.g. about 1:150 v/v in methanol/dichloromethane (2:1 v/v));. An internal standard may be added. E.g. In case the amounts of the at least three biomarkers of panel 1 will be determined, phosphatidylcholine (C19:0 C19:0) dissolved in methanol/dichloromethane (2:1 v/v) may be used as internal standard; for example, 10 μl of respective 0.05 mmolar solution in methanol/dichloromethane (2:1 v/v). Afterwards an extraction is done (e.g. for 5 minutes using a vortexer). After extraction, the sample may be centrifuged (e.g. at about 20.000 g). An aliquot of the supernatant (e.g. 200 μl) could be used for the quantification of the markers. The supernatant may be stored at −20° C. (preferably, up to three months).

In addition, at least one internal standard compound can be added to the sample. As used herein, the term “internal standard compound” refers to a compound which is added to the sample and which is determined (i.e. the amount of the internal standard compound is determined). The at least one internal standard compound can be added before, during, or after extraction of the sample to be tested. In an embodiment, the at least one internal standard compound is added after mixing an aliquot of the sample with the extraction solvent. Preferably, the internal standard compound is thus dissolved in the extraction solvent. In an embodiment, the extraction solvent is a an extraction solvent as described elsewhere herein (such as methanol/dichloromethane (2:1 v/v)). Preferably, the at least one internal standard compound is a lipid metabolite biomarker. More preferably, the internal standard compound is a compound, in particular a lipid, which is essentially not present or which is not present in the sample to be tested. Thus, the compound is preferably not naturally present in the sample to be tested. Preferably, the internal standard is very similar to a respective lipid biomarker according to the present invention. In an embodiment, the at least one standard compound is selected from the group consisting of lysophosphatidylcholine C17:0, ceramide d18:1/17:0, phosphatidylcholine C19:0 C19:0, cholesteryl heptadecanoate (CE C17:0), and glyceryl triheptadecanoate (MMM). In particular, the internal standard compound is phosphatidylcholine C19:0 C19:0.

The internal standard compound(s) can be dissolved in a suitable solvent (the solution comprising the internal standard compound and the suitable solvent is herein also referred to as “internal standard solution”). Preferably, the solvent comprises or is an unpolar liquid solvent, like methylchlorid, dichlormethan, chloroform, 1,2-dichlorethan. Preferably, an extraction solvent as described elsewhere herein is used. Even more preferably the solvent is a mixture comprising dichloromethane (DCM) and methanol. In an embodiment the extraction solvent comprises methanol and DCM, in particular in a ratio of about 2:1 (preferably volume to volume). Preferably, the internal standard solution is added to the sample to be tested after extracting the samples with an extraction solvent as described elsewhere herein and removing the proteins from the sample by centrifugation. Thus, the internal standard solution shall be added to a pretreated sample.

If phosphatidylcholine C19:0 C19:0 is used as internal standard compound, the concentration of the internal standard compound in the internal standard solution to be added to the (pretreated) sample is preferably within a range of 1 to 200 μg/ml, more preferably 35 to 50 μg/ml, most preferably 40 to 45 μg/ml. With regard to the sample after preparation (i.e., as used for the actual measurement), the concentration of the internal standard compound is preferably within a range of 0.01-1.3 μg/ml, more preferably 0.23 to 0.33 μg/ml, most preferably 0.26 to 0.30 μg/ml after sample preparation.

In an embodiment, the volume of the internal standard solution to be used is the same volume as the sample before pretreatment. E.g. 10 μl of plasma and internal standard solution are used.

The determination of the amount of the internal standard shall preferably allow for a normalization of the amount of the at least one biomarker, in particular of the amounts of the at least three biomarkers as referred to herein. Preferably, the determined peak area(s) for the at least one biomarker, in particular of the at least three biomarkers are (is) divided by the peak area of the at least one internal standard compound.

In particular, it is possible to calculate a correction factor for the test samples (samples from subjects to be tested, but also calibration samples). This correction factor can be used in order to correct the peak area of the biomarker(s) for variations of the devices, inherent system errors, or the like. For each biomarker determined as peak area, the area ratio of the biomarker peak area to the internal standard peak area can be determined.

Preferably, the determination of the at least one standard compound does not interfere with the determination of the amount(s) of the at least one biomarker or the at least three biomarkers.

The term “amount” as used herein preferably encompasses the absolute amount of a biomarker as referred to herein, the relative amount or concentration of the said biomarker as well as any value or parameter which correlates thereto or can be derived therefrom. Such values or parameters comprise intensity signal values from specific physical or chemical properties obtained from the said biomarker. In particular, encompassed shall be values or parameters which are obtained by indirect measurements specified elsewhere in this description. It is to be understood that values correlating to the aforementioned amounts or parameters can also be obtained by standard mathematical operations.

In an embodiment, the term “amount” refers to the absolute amount.

In addition, it is contemplated to determine only the amounts of the at least three biomarkers as referred to herein, i.e. no further biomarkers (except for a natriuretic peptide such as NT-proBNP) are determined. For example, if the biomarkers of panel 1 are determined, only the amounts OSS2, PC4 and SM23 are determined (and taken into account for differentiating between heart failure and pulmonary disease, and optionally a natriuretic peptide).Thus, the group of lipid biomarkers to be determined consists of OSS2, PC4 and SM23. If the biomarkers of panel 2 are determined, the group of lipid biomarkers to be determined consists of OSS2; SM23; CE 018:2. If the biomarkers of panel 200 are determined, the group of lipid biomarkers to be determined consists of OSS2; SM23; CE C18:2; PC4.

In a preferred embodiment of the present invention, the method according to the present invention further comprises the determination of amount of a natriuretic peptide, such as the amount of BNP (brain natriuretic peptide, also known as B-type natriuretic peptide) or, in particular, the amount of NT-proBNP (N-terminus of the prohormone brain natriuretic peptide) in a sample from the subject and the thus determined amount of BNP or NT-proBNP is then compared to a reference. In a preferred embodiment the method according to the invention further comprises, in particular in step a) the determination of the amount of BNP (Brain natriuretic peptide, also known as B-type natriuretic peptide) or, in particular, NT-proBNP (N-terminus of the prohormone brain natriuretic peptide) in a sample from the subject. The thus determined amount of BNP or NT-proBNP is then compared to a reference (in step b) for BNP or NT-proBNP. Thus, the at least one or the at least three biomarkers and BNP or NT-proBNP might be determined at the same time (but preferably by varying assays). In another preferred embodiment of the present invention, the amount of NT-proBNP or BNP is determined in a sample in a further step c), and compared to a reference for NT-proBNP or BNP in a further step d). Based on steps b) and d) differentiation of heart failure and pulmonary disease is carried out. Thus, there may be a time gap between the determination of the amounts of the at least one or the at least three biomarkers and the determination of the amount of BNP or NT-proBNP (such as one day or one week).

Alternatively, the amount of NT-proBNP or BNP can be derived from the medical record of the subject to be tested. Thus, the method of the present invention may comprise the step of providing and/or retrieving information on the amount of NT-proBNP or BNP (i.e. the value of this marker).

NT-proBNP and BNP are protein markers. The markers NT-proBNP and BNP are well known in the art. BNP is a 32-amino acid polypeptide secreted by the ventricles of the heart in response to excessive stretching of heart muscle cells (cardiomyocytes). NT-proBNP is a 76 amino acid N-terminal inactive protein that is cleaved from proBNP to release brain natriuretic peptide. BNP is the active hormone and has a shorter half-life than the respective inactive counterpart NT-proBNP. The structure of the human BNP and NT-proBNP has been described already in detail, see e.g., WO 02/089657, WO 02/083913.

It is to be understood that if the protein marker BNP or NT-proBNP is determined, the protein marker is determined in addition to the at least three (metabolite) biomarkers as referred to herein in accordance with the method of the present invention.

The determination of the amount of BNP/NT-proBNP may differ from the determination of the amount of the at least one or at least three biomarkers as referred to in the context of the method of the present invention (since the amount of the at least one biomarker or the amounts of the at least three biomarkers are preferably determined by the methods involving chromatography and mass spectrometry, see elsewhere herein). Preferably, the amount of NT-proBNP or BNP is determined in a blood, serum or plasma sample. Preferably, the amount is determined by using at least one antibody which specifically binds to NT-proBNP or BNP. The at least one antibody forms a complex with the marker to determined (NT-proBNP or BNP). Afterwards the amount of the formed complex is measured. The complex comprises the marker and the antibody (which might be labelled in order to allow for a detection of the complex).

It is to be understood that the sample in which NT-proBNP or BNP is determined requires or may require a pretreatment which differs from the pretreatment of the sample in which the other biomarkers as referred to herein are determined. For example, the proteins comprised by the sample in which this marker is determined are not precipitated. This is taken into account by the skilled person. Preferably, however, the amount(s) of the at least one biomarker or the at least three biomarkers and of BNP or NT-proBNP are measured in aliquots derived from the same sample. Alternatively, the amounts of the at least one biomarker or of the at least three biomarkers and of BNP or NT-proBNP may be measured in aliquots derived from separate samples from the subject.

The further determination of the a natriuretic peptide such as NT-proBNP or BNP allows for a more reliable differentiation.

Instead of NT-proBNP or BNP, or in addition to NT-proBNP or BNP, the present invention further envisages the determination of the amount of ANP (atrial natriuretic peptide) or NT-proANP (N-terminus of the prohormone brain natriuretic peptide). Alternatively, the amount of C-type natriuretic peptide or natriuretic peptide precursor C can be determined. Thus, a natriuretic peptide such as NT-proBNP, BNP, ANP, or NT-proANP can be determined in addition to the at least one or at least three (metabolite) biomarker(s)

In an embodiment, the method of the present invention may further comprise carrying out a correction for confounders. Preferably, the values or ratios determined in a sample of a subject according to the present invention are adjusted for age, BMI, gender or other existing diseases, e.g., the presence or absence of diabetes before comparing to a reference. Alternatively, the references can be derived from values or ratios which have likewise been adjusted for age, BMI, gender. Such an adjustment can be made by deriving the references and the underlying values or ratios from a group of subjects the individual subjects of which are essentially identical with respect to these parameters to the subject to be investigated. Alternatively, the adjustment may be done by statistical calculations. Thus, a correction for confounders may be carried out. Preferred confounders are age, BMI (body mass index) and gender.

In another embodiment, a correction for confounders is not carried out.

The term “reference” in connection with diagnostic methods is well known in the art. The reference in accordance with the present invention shall allow for the differentiation between heart failure and pulmonary disease. A suitable reference may be established by the skilled person without further ado. The reference to be applied may be an individual reference for each of the at least one or at least three biomarkers to be determined in the method of the present invention (and for BNP or NT-proBNP if determined). Accordingly, the amount of each of the at least one or at least three biomarkers (and of BNP or NT-proBNP if determined) as referred to in step a) of the method of the present invention is compared to a reference for each of the at least one or at least three biomarkers (and for BNP or NT-proBNP if determined). For example, if three biomarkers are determined in step a), three references (a reference for the first, a reference for the second, and a reference for the third marker) are applied in step b). A further reference for BNP or NT-proBNP might be used, if the amount of BNP or NT-proBNP is determined in step a). Based on the comparison of the amounts of the at least one or at least three biomarkers (and if BNP or NT-proBNP are determined, the amount of BNP or NT-proBNP), with the references, a differentiation between heart failure and pulmonary disease can be established.

The term “reference” refers to values of characteristic features which allow a differentiation between heart failure and pulmonary disease, i.e. the presence or absence of heart failure, diseases status or an effect referred to herein. Preferably, a reference is a threshold value for the at least one biomarker or the at least three biomarkers as referred to in connection with the present invention whereby values found in a sample to be investigated which are higher than (or depending on the marker lower than) the threshold are indicative for heart failure while those being lower (or depending on the marker higher than) are indicative for pulmonary disease.

The diagnostic algorithm, i.e. the algorithm for the differentiation, may depend on the reference. If the reference amount is e.g. derived from a subject or group of subjects known to suffer from heart failure, the presence of heart failure is preferably indicated by amounts in the test sample which are essentially identical to the reference(s). If the reference amount is e.g. derived from a subject suffering from pulmonary disease or a group thereof but not suffering from heart failure, the presence of heart failure is preferably indicated by amounts of the at least three biomarkers in the test sample which are different from (e.g. increased (“up”) or decreased (“down”) as compared to) the reference(s).

In accordance with the aforementioned method of the present invention, a reference (or references) is, preferably, a reference (or references) obtained from a sample from a subject or group of subjects known to suffer from heart failure. In such a case, a value for the at least one biomarker or a value for each of the at least three biomarkers found in the test sample being essentially identical is indicative for the presence of heart failure. Moreover, the reference, also preferably, could be from a subject or group of subjects known to suffer from pulmonary disease. In such a case, a value for the at least one biomarker or a value for each of the at least three biomarkers found in the test sample being altered with respect to the reference is indicative for the absence of pulmonary disease. Alternatively, a value for the at least one biomarker or a value for each of the at least three biomarkers found in the test sample being essentially identical with respect to the reference is indicative for the presence of pulmonary disease. The same applies mutatis mutandis for a calculated reference, most preferably the average or median, for the relative or absolute value of the the biomarkers of a population of individuals comprising the subject to be investigated. The absolute or relative values of the biomarkers of said individuals of the population can be determined as specified elsewhere herein. How to calculate a suitable reference value, preferably, the average or median, is well known in the art. The population of subjects referred to before shall comprise a plurality of subjects, preferably, at least 5, 10, 50, 100, 1,000 or 10,000 subjects. It is to be understood that the subject to be assessed by the method of the present invention and the subjects of the said plurality of subjects are of the same species.

The value for a biomarker of the test sample and the reference values are essentially identical, if the values for the characteristic features and, in the case of quantitative determination, the intensity values are essentially identical. Essentially identical means that the difference between two values is, preferably, not significant and shall be characterized in that the values for the intensity are within at least the interval between 1^{st} and 99^{th} percentile, 5^{th} and 95^{th} percentile, 10^{th} and 90^{th} percentile, 20^{th} and 80^{th} percentile, 30^{th} and 70^{th} percentile, 40^{th} and 60^{th} percentile of the reference value, preferably, the 50^{th}, 60^{th}, 70^{th}, 80^{th}, 90^{th} or 95^{th} percentile of the reference value. Statistical test for determining whether two amounts or values are essentially identical are well known in the art and are also described elsewhere herein.

An observed difference for two values, on the other hand, shall be statistically significant. A difference in the relative or absolute value is, preferably, significant outside of the interval between 45^{th} and 55^{th} percentile, 40^{th} and 60^{th} percentile, 30^{th} and 70^{th} percentile, 20^{th} and 80^{th} percentile, 10^{th} and 90^{th} percentile, 5^{th} and 95^{th} percentile, 1st and 99^{th} percentile of the reference value.

In a preferred embodiment the value for the characteristic feature can also be a calculated out-put such as score of a classification algorithm like “elastic net” as set forth elsewhere herein.

Preferably, the reference, i.e. a value or values for at least one characteristic feature (e.g. the amount) of the biomarkers or ratios thereof, will be stored in a suitable data storage medium such as a database and are, thus, also available for future assessments.

The term “comparing”, preferably, refers to determining whether the determined value of the biomarkers, or score (see below) is essentially identical to a reference or differs therefrom. Preferably, a value for a biomarker (or score) is deemed to differ from a reference if the observed difference is statistically significant which can be determined by statistical techniques referred to elsewhere in this description. If the difference is not statistically significant, the biomarker value and the reference are essentially identical. Based on the comparison referred to above, a subject can be assessed to suffer from heart failure (in particular, from heart failure with reduced ejection fraction or another subform of heart failure), or pulmonary disease.

For the specific biomarkers referred to in this specification, preferred values for the changes in the relative amounts or ratios (i.e. the changes expressed as the ratios of the means) or the kind of direction of change (i.e. “up”-or “down” or increase or decrease resulting in a higher or lower relative and/or absolute amount or ratio) are indicated in the Table 1 in the Examples section. The ratio of means indicates the degree of increase or decrease, e.g., a value of 2 means that the amount is twice the amount of the biomarker compared to the reference. Moreover, it is apparent whether there is an “up-regulation” or a “down-regulation”. In the case of an “up-regulation” the ratio of the mean shall exceed 1.0 while it will be below 1.0 in case of a “down”-regulation. Accordingly, the direction of regulation can be derived from the Table as well. It will be understood that instead of the means, medians could be used as well.

E.g. with respect to the biomarkers of panel 1, heart failure is preferably diagnosed in the subject when the amount of OSS2 in the sample is greater than the reference and the amount of SM23 in the sample is less than the reference, and the amount of PC4 in the sample is less than the reference.

As it can be derived from the last column in Table 1 in the Examples section, the amount of the individual biomarkers in heart failure patients might be unchanged as compared to the amount in patients with pulmonary disease. Nevertheless, the combined determination of the amounts of the marker combinations as referred to herein, the subsequent calculation of a score based on these amounts, and the comparison with a reference score allowed for carrying out the method of the present invention. For the reference score, see elsewhere herein.

If the amount of NT-proBNP or BNP is determined, an increased amount of this marker shall be indicative for the presence of heart failure, whereas a decreased or an essentially identical amount shall be indicative for the absence of heart failure (and thus for the presence of pulmonary disease). In an embodiment, the reference amount for NT-proBNP is about 125 μg/mL, preferably in a serum or plasma sample, in particular for a human subject. However, further references may be used (e.g. since the level can depend on the age).

The phrase “presence of heart failure” or “indicative for heart failure” also means that the shortness of breath has been caused by heart failure. The phrase “presence of pulmonary disease” or “indicative for pulmonary disease” also means that the shortness of breath has been caused by pulmonary disease.

It is to be understood that the diagnostic algorithm, i.e. the algorithm used for the differentiation, might depend on the reference or references to be applied. However, this is taken into account by the skilled person who can establish suitable reference values and/or diagnostic algorithms based on the differentiation provided herein. For example, the reference might be derived from a subject or group of subjects known to suffer from heart failure. In this case the following applies: With respect to triacylglyceride biomarker, an amount (or amounts) of the triacylglyceride(s) in the sample from the subject which is (are) essential identical or which is (are) increased as compared to the reference is indicative for the presence of heart failure. With respect to the remaining biomarkers that can determined, an amount (or amounts) of the remaining biomarker(s) in the sample from the subject which is (are) decreased or essentially identical as compared to the reference is (are) indicative for the presence of heart failure.

The comparison is, preferably, assisted by automation. For example, a suitable computer program comprising algorithms for the comparison of two different data sets (e.g., data sets comprising the values of the characteristic feature(s)) may be used. Such computer programs and algorithms are well known in the art. Notwithstanding the above, a comparison can also be carried out manually.

In the context of step b) of the present invention, the amounts of a group of biomarkers as referred to in step a) of the methods of the present invention shall be compared to a reference or references. Thereby, it can be differentiated between pulmonary disease and heart failure (in particular as the cause for shortness of breath). In an embodiment references for the individual determined biomarkers, i.e. references for each of biomarkers as referred to in step a) are applied. However, it is also envisaged to calculate a score (in particular a single score) based on the amounts of the at least three biomarkers as referred to in step a) of the method of the present invention, i.e. a single score, and to compare this score to a reference score. Preferably, the score is based on the amounts of the at least three biomarkers in the sample from the test subject, and, if NT-proBNP or BNP is determined, on the amounts of the at least three biomarkers and the amount of NT-proBNP or BNP in the sample from the test subject. For example, if the amounts of the biomarkers of panel 1 are determined, the calculated score is based on the amounts of SM23, OSS2 and PC4 in the sample from the test subject. If additionally NT-proBNP is determined, the score is based on the amount of SM23, OSS2, PC4 and NT-proBNP of the test subject.

The calculated score combines information on the amounts of the at least three biomarkers. Moreover, in the score, the biomarkers are, preferably, weighted in accordance with their contribution to the establishment of the differentiation. Based on the combination of biomarkers applied in the method of the invention, the weight of an individual biomarker may be different.

The score can be regarded as a classifier parameter for differentiating in a subject between heart failure and pulmonary disease. In particular, it enables the person who provides the differentiation based on a single score. The reference score is preferably a value, in particular a cut-off value which allows for differentiating between heart failure and pulmonary disease in the subject to be tested. Preferably, the reference is a single value. Thus, the person does not have to interpret the entire information on the amounts of the individual biomarkers.

Using a scoring system as described herein, advantageously, values of different dimensions or units for the biomarkers may be used since the values will be mathematically transformend into the score. Accordingly, e.g. values for absolute concentrations may be combined in a score with peak area ratios.

The reference score to be applied may be elected based on the desired sensititvy or the desired specificity. How to elect a suitable reference score is well known in the art. For example, six different cut-offs (reference scores) were determined in Example 6 of the Examples section.

Thus, in a preferred embodiment of the present invention, the comparison of the amounts of the biomarkers to a reference as set forth in step b) of the method of the present invention encompasses step b1) of calculating a score based on the determined amounts of the biomarkers as referred to in step a), and step b2) of comparing the, thus, calculated score to a reference score. More preferably, a logistic regression method is used for calculating the score and, most preferably, said logistic regression method comprises elastic net regularization.

Alternatively, the amount of each of the at least three biomarkers is compared to a reference, wherein the result of this comparison is used for the calculation of a score (in particular a single score), and wherein said score is compared to a reference score.

Thus, the present invention, in particular, a method for differentiating between heart failure and pulmonary disease in a subject comprising the steps of:
a) determining in a sample of a subject as referred to herein the amounts of at least three biomarkers as referred to above; and
b1) calculating a score based on the determined amounts of the at least three biomarkers as referred to in step a), and
b2) comparing the, thus, calculated score to a reference score, whereby it is differentiated in a subject between heart failure and pulmonary disease.

As set forth elsewhere herein, the aforementioned method may further comprise in step a) the determination of the amount of BNP or NT-proBNP. The amount of BNP or NT-proBNP may contribute to the score calculated in step b). Accordingly, the method comprises the following steps:
a) determining in a sample of a subject as referred to herein the amounts of at least three biomarkers as referred to above and the amount of BNP or NT-proBNP; and
b1) calculating a score based on the determined amounts of the at least three biomarkers and on the amount of BNP or NT-proBNP as referred to in step a), and
b2) comparing the, thus, calculated score to a reference score, whereby it is differentiated in a subject between heart failure and pulmonary disease.

As set forth elsewhere herein, the amount of NT-proBNP or BNP can be also derived from the medical record of the subject to be tested. In this case, it is not required to determine the amount of this marker in step a).

Alternatively, the amount of BNP or NT-proBNP may not contribute to the score calculated in step b1). Accordingly, the method comprises the following steps:
a) determining in a sample of a subject as referred to herein the amounts of at least three biomarkers as referred to above and the amount of BNP or NT-proBNP; and
b1) calculating a score based on the determined amounts of the at least three biomarkers, and
b2) comparing the, thus, calculated score to a reference score, and comparing the amount of BNP or NT-proBNP to a reference, whereby it is differentiated in a subject between heart failure and pulmonary disease.

As set forth elsewhere herein, the amount of NT-proBNP or BNP can be also derived from the medical record of the subject to be tested. In this case, it is not required to the determine the amount of this marker in step a).

Preferably, the reference score shall allow for differentiating whether a subject suffers from heart failure or pulmonary disease. Preferably, the differentiation is made by assessing whether the score of the test subject is above or below the reference score. In an embodiment, a score above the reference score is indicative for heart failure, whereas a score below the reference score is indicative for pulmonary disease.

Thus, in an embodiment it is not necessary to provide an exact reference score. A relevant reference score can be obtained by correlating the sensitivity and specificity and the sensitivity/specificity for any score, preferably, using ROC-curve-analysis. A reference score resulting in a high sensitivity results in a lower specificity and vice versa. Thus, the reference score may depend on the desired sensitivity and/or specificity. Preferably, the reference score is based on the same markers (e.g. the at least three biomarkers and NT-proBNP) as the score.

The reference score may be a “Cut-Off” value which allows for differentiating between the presence and the absence of heart failure in the subject.

In accordance with the present invention, a reference score is, preferably, a reference score obtained from a sample from a subject or group of subjects known to suffer from heart failure. In such a case, a score in the test sample being essentially identical is indicative for the presence of the disease, i.e. of heart failure. Moreover, the reference score, also preferably, could be from a subject or group of subjects known to suffer from pulmonary disease. In such a case, a score in the test sample being altered, in particular decreased, with respect to the reference score is indicative for the presence of pulmonary disease. Alternatively, a score in the test sample being essentially identical to said reference score is indicative for the presence of pulmonary disease.

Preferably, the score is calculated based on a suitable scoring algorithm. Said scoring algorithm, preferably, shall allow for differentiating whether a subject suffers from a disease as referred to herein, or not, based on the amounts of the biomarkers to be determined.

Preferably, said scoring algorithm has been previously determined by comparing the information regarding the amounts of the individual biomarkers as referred to in step a) in samples from patients suffering from heart failure as referred to herein and from patients suffering from pulmonary disease. Accordingly, step b) may also comprise step b0) of determining or implementing a scoring algorithm. Preferably, this step is carried out prior steps b1) and b2).

In an embodiment the reference score is calculated such that an increased value of the score of the test subject as compared to the reference score is indicative for the presence of heart failure, and/or a decreased value of the score of the test subject as compared to the reference score is indicative for the presence of pulmonary disease. In particular, the score may be a cut-off value.

In a preferred embodiment of the present invention (e.g. of the methods, devices, uses etc.), the reference score is a single cut-off value. Preferably, said value allows for allocating the test subject either into a group of subjects suffering from heart failure or a into a group of subjects suffering from pulmonary disease. Preferably, a score for a subject lower than the reference score is indicative for the presence of pulmonary disease in said subject, whereas a score for a subject larger than the reference score is indicative for the presence of heart failure in said subject.

In another preferred embodiment of the present invention (e.g. of the methods, devices, uses etc.), the reference score is a reference score range. In this context, a reference score range indicative for the presence of heart failure, a reference score range indicative for the presence of pulmonary disease, or two reference score ranges (i.e. a reference score range indicative for the presence of heart failure and a reference score range indicative for the presence of pulmonary disease) can be applied.

A suitable scoring algorithm can be determined with the at least three biomarkers referred to in step a) of the method of the present invention by the skilled person without further ado (and optionally of BNP or NT-proBNP). E.g., the scoring algorithm may be a mathematical function that uses information regarding the amounts of the at least three biomarkers (and optionally of BNP or NT-proBNP) in a cohort of subjects suffering from heart failure and a cohort of subjects suffering from pulmonary disease. Methods for determining a scoring algorithm are well know in the art and including Significance Analysis of Microarrays, Tree Harvesting, CART, MARS, Self Organizing Maps, Frequent Item Set, Bayesian networks, Prediction Analysis of Microarray (PAM), SMO, Simple Logistic Regression, Logistic Regression, Multilayer Perceptron, Bayes Net, Naïve Bayes, Naïve Bayes Simple, Naïve Bayes Up, IB1, lbk, Kstar, LWL, AdaBoost, ClassViaRegression, Decorate, Multiclass Classifier, Random Committee, j48, LMT, NBTree, Part, Random Forest, Ordinal Classifier, Sparse Linear Programming (SPLP), Sparse Logistic Regression (SPLR), Elastic net, Support Vector Machine, Prediction of Residual Error Sum of Squares (PRESS), Penalized Logistic Regression, Mutual Information. Preferably, the scoring algorithm is determined with or without correction for confounders as set forth elsewhere herein.

Further, a suitable scoring algorithm can be determined with the at least one (metabolite)biomarker and of BNP or NT-proBNP by the skilled person without further ado.

In an embodiment, the scoring algorithm is determined with an elastic net with at least three biomarkers and optionally with BNP or NT-proBNP (see also Examples section).

Typically, a classification algorithm such as those implementing the elastic net method may be used for scoring (Zou 2005, Journal of the Royal Statistical Society, Series B: 301-320, Friedman 2010, J. Stat. Sotw. 33). Thus, the score for a subject can be, preferably, calculated with a logistic regression model fitted, e.g., by using the elastic net algorithm such as implemented in the R package glmnet. More specifically, the score may be calculated by the following formula

$$p = \frac{1}{1 + {e}^{- ( {w}_{0} + {\sum }_{i = 1}^{n} {w}_{i} {\hat{x}}_{i} )}}$$

or a mathematically equivalent formula,

with the feature \hat{x}ᵢ being

$${\hat{x}}_{i} = \frac{{x}_{i} - {m}_{i}}{{s}_{i}}$$

wherein xᵢ are the log-transformed measurement values, e.g., peak area ratios and/or concentration values, and mᵢ, sᵢ are feature specific scaling factors and wᵢ are the coefficients of the model w₀, intercept; , coefficient for the first feature (e.g. NT-proBNP, or any one of the lipid biomarkers as referred to herein); w₂ . . . wₙ, coefficients for the further features; , number of feautures in the panel].

A score larger than the reference score is indicative for a subject who suffers from heart failure whereas a score lower than (or equal to) the reference score is indicative for a subject who suffers from pulmonary disease.

The reference scores e.g. can be determined to maximize the Youden index for the detection of heart failure.

A further preferred method to calculate the score and the reference score is described in the Examples section. Moreover, Examples 4 and 5 disclose a preferred reference score for panel 1. For example, the reference score for panel 1 in combination with NT-proBNP may be 0.738.

The amounts of the at least three biomarkers referred to above shall allow for the differentiation between heart failure and pulmonary disease. Accordingly, the at least three biomarkers as specified above in a sample can, in principle, be used for differentiating between heart failure and pulmonary disease. This is particularly helpful for an efficient differentiation as well as for improving of the pre-clinical and clinical management of patients, in particular of patients suffering from shortness of breath.

The definitions and explanations of the terms made above apply mutatis mutandis for the following embodiments of the present invention (e.g. to the kits, devices, uses, further methods etc.) except specified otherwise herein below.

By carrying out the method of the present invention, a subject can be identified who is in need for a therapy of heart failure or in need for a therapy of pulmonary disease. Accordingly, the present invention relates to a method for identifying whether a subject is in need for a therapy of heart failure or in need for a therapy of pulmonary disease. Accordingly, the present invention preferably relates to a method for identifying whether a subject suffering from shortness of breath is in need for a therapy of heart failure or in need for a therapy of pulmonary disease.

The phrase “in need for a therapy of heart failure” or “in need for a therapy of pulmonary disease” as used herein means that the disease in the subject is in a status where therapeutic intervention is necessary or beneficial in order to ameliorate or treat heart failure or pulmonary disease or the symptoms associated therewith. Accordingly, the findings of the studies underlying the present invention do not only allow for differentiating in a subject between heart failure and pulmonary disease but also allow for identifying subjects which should be treated by a heart failure therapy or a pulmonary disease therapy. Once the subject has been identified, the method may further include a step of making recommendations for a therapy.

A therapy of heart failure as used in accordance with the present invention, preferably, relates to a therapy which comprises or consists of the administration of at least one drug selected from the group consisting of: ACE Inhibitors (ACEI), Beta Blockers, AT1-Inhibitors, Aldosteron Antagonists, Renin Antagonists, Diuretics, Ca-Sensitizer, Digitalis Glykosides, antiplatelet agents, Vitamin-K-Antagonists, polypeptides of the protein S100 family (as disclosed by DE000003922873A1, DE000019815128A1 or DE000019915485A1 hereby incorporated by reference), natriuretic peptides such as BNP (Nesiritide (human recombinant Brain Natriuretic Peptide-BNP)) or ANP.

As a rule, patients are preferably treated with medication as recommended by the guidelines of the European Society of Cardiology (Ref: European Heart Journal (2012), 33:1787-1847).

In another embodiment the patients suffering from heart failure are treated as recommended by the 2013 ACCF/AHA guidelines (see Circulation. 2013; 128: e240-e327).

Moreover, the subject suffering from heart failure may be treated with angiotensin receptor blockers (ARBs), ivabradine, digoxin and other digitalis glycosides, hydralazine and isosorbide dintrate (vasodilators) and omega-3 polyunsaturated fatty acids.

In a preferred embodiment of the method of the invention, the determination of the at least one biomarker or the at least three biomarkers is achieved by mass spectroscopy techniques (preferably GC-MS and/or LC-MS), NMR or others referred to herein above. In such cases, preferably, the sample to be analyzed is pretreated. Said pretreatment, preferably, includes obtaining of the at least one preferably the at least three biomarker from sample material, e.g., plasma or serum may be obtained from whole blood or the at least one, preferably the at least three biomarkers may even be specifically extracted from sample material. Moreover, for GC-MS, further sample pretreatment such as derivatization of the at least one biomarker is, preferably, required. Furthermore, pretreatment also, preferably, includes diluting sample material and adjusting or normalizing the concentration of the components comprised therein. To this end, preferably, normalization standards may be added to the sample in predefined amounts which allow for making a comparison of the amount of the at least one biomarker and the reference and/or between different samples to be analyzed. Preferably, the normalization standard is an internal standard compound as defined elsewhere herein. For example, one standard for each class of biomarkers may be added in order to allow for a normalization, e.g one standard for triacylglyerides, one standard for sphingomyelins etc. In another preferred embodiment the quantification of SM biomarker is achieved by adding commercially available sphingomyeline standards with a different chain length than the target metabolites based on the observation that the detector response is the same. In a further preferred embodiment the calibration solutions are prepared in delipidized plasma (commercially available) to simulate a matrix as close as possible to real plasma.

Accordingly, the present invention further pertains to the use of a composition or kit comprising the at least one or the at least three biomarkers as a control when carrying out the present invention.

In addition, the present invention relates to use a calibration solution when carrying out the method of the present invention, said calibration solution comprising said composition comprising said at least one or said at least three biomarkers. The calibration solution shall allow for the calibration of the device used for the determination of the amounts of the at least one biomarker or the at least three biomarkers. Preferably, the calibration solution serves as stock solution for the preparation of a calibration sample/calibration samples which are defined herein below. Preferably, the at one biomarker or the at least three biomarkers are dissolved in a suitable solvent to form together the calibration solution. Thus, the calibration solution preferably comprises a suitable solvent and said composition comprising said at least three biomarkers. Preferably, the solvent comprises or is an unpolar liquid solvent, like methylchlorid, dichlormethan, chloroform, 1,2-dichlorethan. Preferably, an extraction solvent as described elsewhere herein is used. Even more preferably the solvent is a mixture comprising dichloromethane (DCM) and methanol. In an embodiment the extraction solvent comprises methanol and DCM, in particular in a ratio of about 2:1 (preferably volume to volume).

Furthermore, the present invention relates to the use of a calibration sample comprising the composition of the present invention (i.e. comprising at least three biomarkers as set forth in connection with the method of the present invention), and delipidized serum or plasma. Preferably, the calibration sample further comprises a suitable solvent, in particular an extraction sol vent as defined elsewhere herein.

Said composition, calibration solution or said calibration sample can be used as control(s) when carrying out the present invention, in particular, for controlling and/or calibrating the device(s) for the determination of the amount of the at least three biomarkers.

In a preferred embodiment, the delipidized serum or plasma is defibrinated. In an embodiment said delipidized sample is delipidized serum. In another embodiment said delipidized sample is delipidized plasma.

Preferably, said composition, calibration solution, or calibration sample comprises the at least three biomarkers in predefined amounts or ratios.

The term “delipidized” (frequently also referred to as “delipidated”) is well known in the art. Preferably, the term means that the lipids (such as triglycerides, cholesterols, phospholipids, and unesterified fatty acids) that are naturally present in said sample (e.g. serum, plasma) have been removed from said sample (see e.g. Cham et al. J Lipid Res. 1976 March; 17(2):176-81. A solvent system for delipidation of plasma or serum without protein precipitation). After delipidizing said sample, the at least three biomarkers are referred herein are added, in particular artificially added, to said delipidized serum or plasma. Thus, the at least three biomarkers are preferably spiked into said delipidized sample. Preferably, the at least three biomarkers are added to the delipidized serum or plasma in predefined amounts. Preferably, said predefined amounts shall allow for a calibration and/or control. Alternatively, said predefined amounts shall represent a reference.

In order to add the at least one biomarker or the at least three biomarkers to the delipidized serum or plasma, the at least one or three biomarker(s) are dissolved in a suitable solvent (which is in particular the extraction solvent as defined elsewhere herein). The resulting solution is then added, i.e. combined with delipidized serum or plasma. Preferably, the ratio of the delipidized serum or plasma to the solvent, is about 1:10 to 1:1000 more preferably, about 1:100: to 1:500, or even more preferably about 1:100 to 1:200 and most preferably about 1:150.

For the calibration, an aliquot of the calibration solution can be mixed with delipidized serum or plasma, thereby producing the calibration sample or a series of calibrations samples. Preferably, the further calibration samples may thus prepared be from a stock solution being a calibraton solution with the highest concentration of the biomarkers. In case an internal standard is added, preferably the same amount of internal standard is given to each member of the series of calibration samples (see below).

In a preferred embodiment of the present invention, the composition, the calibration solution, the calibration sample, or kit comprises the lipid biomarkers that are used for the differentiation (i.e. the same combination of lipid biomarkers for example the biomarkers of panels 1, 2 or 200).

However, it is envisaged that the lipid biomarkers are replaced with other biomarkers, said other biomarker preferably belonging to the same compound class. Thus, a triacylglyceride biomarker may be replaced with a different triacylglyceride, a cholesterylester biomarker may be replaced with a different cholesterylester, a phosphatidylcholine biomarker may be replaced with a different phosphatidylcholine, a sphingomyelin biomarker may be replaced with a different sphingomyelin, a ceramide biomarker may be replaced with a different ceramide. For example, if the method of the present invention comprises the determination of SM23, the delipidized sample may comprise SM27 instead. SM27 preferably refers to Sphingomyelin (d18:1/24:1) or Sphingomyelin(d18:2/24:0), or a combination thereof. In a preferred embodiment, SM27 is Sphingomyelin (d18:1/24:1). Preferred triacylglyceride, cholesterylester, phosphatidylcholine, ceramide, and sphingomyelin are described elsewhere herein.

The composition, calibration solution, calibration sample, or kit as defined herein thus preferably comprises
i. at least one triacylglyceride, at least one cholesterylester, and at least one phosphatidylcholine;
ii. at least one triacylglyceride, at least one phosphatidylcholine, and at least one sphingomyelin;
iii. at least one triacylglyceride, at least one cholesterylester, and at least one sphingomyelin; or
iv. at least one phosphatidylcholine, at least one cholesterylester, and at least one sphingomyelin;

In another preferred embodiment, the composition, calibration solution, calibration sample, or kit may comprise at least one triacylglyceride, at least one cholesterylester, at least one sphingomyelin and at least one phosphatidylcholine.

In a particular preferred embodiment, the composition, calibration solution, calibration sample, or kit comprises OSS2, PC4, and SM23. In another particular preferred embodiment, the composition, calibration solution, or calibration sample comprises OSS2, PC4, and SM27.

In a preferred embodiment, in particular for Panel 1, the ratios of PC4: SM27 (based on molar concentrations) is in the range of 50:1: to 2,5:1, in a more preferred embodiment the ratio is in the range of 25:1 to 5:1. In another preferred embodiment, in particular for Panel 1, the ratios of SM27: OSS2 (based on molar concentrations) is in the range of 30:1: to 1.5:1, in a more preferred embodiment the ratio is in the range of 15:1 to 2:1. In particular the ratios of of PC4: SM27: OSS2 (based on molar concentrations) are about 100:7.9:1.1. In case SM23 is part of the composition, calibration solution, calibration sample, or kit instead of SM27 the same ratios as mentioned before apply

In another particular preferred embodiment, the composition, calibration solution, calibration sample, or kit comprises OSS2, CE 18:2, and SM23. In another particular preferred embodiment, the composition, calibration solution, or calibration sample comprises OSS2, CE 18:2, and SM27. In a preferred embodiment, in particular for Panel 2, the ratios of CE18:2: SM27 (based on molar concentrations) is in the range of 500:1: to 2:1, in a more preferred embodiment the ratio is in the range of 100:1 to 10:1. In another preferred embodiment, in particular for for Panel 2, the ratios of SM27: OSS2 (based on molar concentrations) is in the range of 30:1: to 1.5:1, in a more preferred embodiment the ratio is in the range of 15:1 to 2:1. In particular the ratios of of CE 8:2:SM27:OSS2 (based on molar concentrations) are about 100:2.7:0.39. In case SM23 is part of the composition, calibration solution, calibration sample, or kit instead of SM27 the same ratios as mentioned before apply

In another particular preferred embodiment, the composition, calibration solution, calibration sample, or kit comprises OSS2, PC4, SM23 and CE18:2. In another particular preferred embodiment, the composition, calibration solution, or calibration sample comprises OSS2, PC4, CE 18:2 and SM27.In a preferred embodiment,in particular for Panel 200, the ratios of PC4: SM27 (based on molar concentrations) is in the range of 50:1: to 2.5:1, in a more preferred embodiment the ratio is in the range of 25:1 to 5:1. In another preferred embodiment, in particular for Panel 200, the ratios of SM27: OSS2 (based on molar concentrations) is in the range of 30:1: to 1.5:1, in a more preferred embodiment the ratio is in the range of 15:1 to 2:1. In another preferred embodiment the ratios of CE18:2: SM27 (based on molar concentrations) is in the range of 500:1: to 2:1, in a more preferred embodiment the ratio is in the range of 100:1 to 10:1. In particular the ratios of CE18:2:PC4:SM27:OSS2 (based on molar concentrations) are about 100:34.2:2.7:0.4. In case SM23 is part of the composition, calibration solution, calibration sample, or kit instead of SM27 the same ratios as mentioned before apply

In addition, the present invention relates to a series of calibration solutions and/or calibration samples and/or compositions of the present invention. Preferably, said series of calibration solutions/calibration samples comprises at least three, more preferably at least four and most preferably at least five different calibration solutions and/or calibration samples and/or compositions. Said calibration solutions and/or calibration solutions and/or compositions shall differ in the predefined amounts of the lipid biomarkers comprised by said solutions/samples/compositions. Thus, the present invention envisages a dilution series of calibration solutions and/or calibration samples of the present invention. In an embodiment, the amounts of the biomarkers comprised by the series of calibration solutions, and/or calibration samples are in a linear relationship so that the highest concentrated one can be used as stock solution.

Preferred amounts of the biomarkers PC4, CE 18:2, SM27 and OSS2 present in the calibration solution(s) are shown in below. Table 13 of the Examples section. Tables 5 and 5A list the stock solutions used for the calibration. Preferred ratios of the biomarkers are shown in Table 5 for panel 200, panel 2, and panel 1 (see in particular column STD1). Preferably, the calibration solution comprises OSS2, PC4, and/or SM27. Preferably, the concentration is a follows:

PC4: from about 6 to about 193 nmol/ml

OSS2: from about 0.07 to about 2.2 nmol/ml, and/or

SM27: from about 0.5 to about 15,2 nmol/ml

If the calibration solution comprises CE 18:2, the concentration is preferably from about 18 to about 570 nmol/ml.

In an embodiment, a calibration solution or calibration sample comprising PC4, OSS2 and SM27 is used, if the biomarkers of panel 1 are determined. In another embodiment, a calibration solution or calibration sample comprising PC4, OSS2 and SM23 is used, if the biomarkers of panel 1 are determined.

In an embodiment, a calibration solution or calibration sample or kit comprising OSS2, CE 18:2 and SM27 is used, if the biomarkers of panel 2 are determined. In another embodiment, a calibration solution or calibration sample or kit comprising OSS2, CE 18:2 and SM23 is used, if the biomarkers of panel 2 are determined.

In an embodiment, a calibration solution or calibration sample or kit comprising PC4, OSS2, CE 18:2 and SM27 is used, if the biomarkers of panel 200 are determined. In another embodiment, a calibration solution or calibration sample comprising PC4, OSS2, CE 18:2 and SM23 or kit is used, if the biomarkers of panel 200 are determined. However, it can also be envisaged that this calibration solution or calibration sample or kit can be used, if the biomarkers of panel 1 or panel 2 are determined.

Further preferred calibration solutions are described in the examples section, see, e.g., Example 3.

As set forth above, the composition, calibration solution, calibration sample or kit shall comprise the at least three biomarkers. It is to be understood, that the at least three biomarkers do not have to be comprised by the same calibration solution or calibration sample. So a plurality of calibration solutions, or calibration samples can be prepared. Rather, they can be comprised in individual calibration solutions or calibration samples. Thus, if three biomarkers (Marker A, Marker B and Marker C) shall be determined, the individual biomarkers can be comprised in one calibration solution/sample with markers A, B, and C, two calibration solutions/samples (one with A and B, one with C; or one with A and C, and one with B; one with A and C and one with B), or three calibration solutions/samples.

Thus, the present invention relates to a kit comprising i) a single calibration solution, said single calibration solution comprising the at least three biomarkers as set forth in connection with the method of the present invention or ii) a plurality of calibration solutions, said plurality of calibration solutions comprising the at least three biomarkers as set forth in connection with the method of the present invention, wherein preferably in the plurality of calibration solutions each calibration solution comprises at least one of the said at least three biomarkers. More preferably, the calibration solutions in the plurality of calibration solutions do not comprise identical biomarkers. In a preferred embodiment, the at least three biomarkers are the biomarkers of panel 1, 2 or 200, wherein SM23 has been replaced by SM27. E.g, with respect to panel 1, the kit comprises the biomarkers OSS2, PC4, and SM23 or OSS2, PC4, and SM27. Preferred concentrations or ratios for the biomarkers of some panels are described above. In addition, the kit may comprise an the internal standard solution of the present invention. Preferably, the internal standard solution comprises phosphatidylcholine C19:0 C19:0.

In a preferred embodiment of the composition, the calibration solution, the calibration sample, or kit of the present invention, the composition, the calibration solution, the calibration sample, or kit further comprises one or more internal standard compounds. Preferably, the composition, the calibration solution, the calibration sample, or kit further comprises phosphatidylcholine C19:0 C19:0. In an embodiment of the present invention for Panels 1, 2 and/or 200 the calibration sample with the highest concentration of the respective biomarkers, comprises OSS2 and phosphatidylcholine C19:0 C19:0 (based on molar concentrations) in an ratio of 0.05:1 to 2:1, in a preferred embodiment the ratio is in the range of 0.1:1 to 0.5 to 1. In particular for panel 1 the ratios (based on molar concentrations) of PC4:SM27:0SS2:phosphatidylcholine C19:0 C19:0 is about 100:7.9:1.1:5.2. The same ratios apply if instead of SM 27 SM23 is used.

The present invention also relates to an internal standard solution as described herein above. Preferably, the internal standard solution comprises at least one internal standard compound. In an embodiment, the internal standard compound is phosphatidylcholine C19:0 C19:0. Preferably, the at least one internal standard compound is dissolved in a suitable solvent to form together the internal standard solution. Thus, the internal standard solution preferably comprises a suitable solvent and said composition comprising said at least three biomarkers. Preferably, the solvent comprises or is an unpolar liquid solvent, like methylchlorid, dichlormethan, chloroform, 1,2-dichlorethan. Preferably, an extraction solvent as described elsewhere herein is used. Even more, preferably the solvent is a mixture comprising dichloromethane (DCM) and methanol. In an embodiment the extraction solvent comprises methanol and DCM, in particular in a ratio of about 2:1 (preferably volume to volume).

The method of the present invention, in a preferred embodiment, furthermore further comprises a step of recommending and/or managing the subject according to the result of the differentiation established in step b). Such a recommendation may, in an aspect, be an adaptation of life style, nutrition and the like aiming to improve the life circumstances, the application of therapeutic measures as set forth elsewhere herein in detail, and/or a regular disease monitoring.

In another preferred embodiment of the aforementioned method, step b) is carried out by an evaluation unit as set forth elsewhere herein.

Further, the present invention also in an aspect pertains to a method of treating heart failure comprising the steps a) and b) of the method for differentiating in a subject between heart failure and pulmonary disease, and the further step c) of treating the subject in case the subject with heart failure therapy or pulmonary disease therapy.

The definitions and explanations of the terms made above apply mutatis mutandis for the following embodiments of the present invention except specified otherwise herein below.

The aforementioned methods for the determination of the at least one or the at least three biomarker(s) can be implemented into a device. A device as used herein shall comprise at least the aforementioned means. Moreover, the device, preferably, further comprises means for comparison and evaluation of the detected characteristic feature(s) of the at least one or the at least three biomarker(s) and, also preferably, the determined signal intensity. The means of the device are, preferably, operatively linked to each other. How to link the means in an operating manner will depend on the type of means included into the device. For example, where means for automatically qualitatively or quantitatively determining the biomarker are applied, the data obtained by said automatically operating means can be processed by, e.g., a computer program in order to facilitate the assessment. Preferably, the means are comprised by a single device in such a case. Said device may accordingly include an analyzing unit for the biomarker and a computer unit for processing the resulting data for the assessment. Preferred devices are those which can be applied without the particular knowledge of a specialized clinician, e.g., electronic devices which merely require loading with a sample.

Alternatively, the methods for the determination of the at least one biomarker can be implemented into a system comprising several devices which are, preferably, operatively linked to each other. Specifically, the means must be linked in a manner as to allow carrying out the method of the present invention as described in detail above. Therefore, operatively linked, as used herein, preferably, means functionally linked. Depending on the means to be used for the system of the present invention, said means may be functionally linked by connecting each mean with the other by means which allow data transport in between said means. A preferred system comprises means for determining biomarkers. Means for determining biomarkers as used herein encompass means for separating biomarkers, such as chromatographic devices, and means for metabolite determination, such as mass spectrometry devices. Suitable devices have been described in detail above. Preferred means for compound separation to be used in the system of the present invention include chromatographic devices, more preferably devices for liquid chromatography, HPLC, and/or gas chromatography. Preferred devices for compound determination comprise mass spectrometry devices, more preferably, GC-MS, LC-MS, direct infusion mass spectrometry, FT-ICR-MS, CE-MS, HPLC-MS, quadrupole mass spectrometry, sequentially coupled mass spectrometry (including MS-MS or MS-MS-MS), ICP-MS, Py-MS or TOF. The separation and determination means are, preferably, coupled to each other. Most preferably, LC-MS, in particular HPLC-MS, and/or GC-MS are used in the system of the present invention as described in detail elsewhere in the specification. Further comprised shall be means for comparing and/or analyzing the results obtained from the means for determination of the at least one biomarker or the least three biomarker(s) (and optionally of NT-proBNP or BNP). The means for comparing and/or analyzing the results may comprise at least one databases and an implemented computer program for comparison of the results. Preferred embodiments of the aforementioned systems and devices are also described in detail below.

Therefore, the present invention relates to a diagnostic device comprising:
a) an analysing unit comprising at least one detector for the at least one biomarker, in particular for the at least three biomarkers as referred to herein in connection with the present invention detected by the at least one detector, and, operatively linked thereto;
b) an evaluation unit comprising a computer comprising tangibly embedded a computer program code for carrying out a comparison of the determined amount of the at least one biomarker, in particular the determined amounts at least three biomarkers and the reference amount(s), and a data base comprising said reference amount(s) for the said biomarker, whereby it is differentiated between heart failure and pulmonary disease.

Alternatively, the evaluation unit under b) comprises a computer comprising tangibly embedded a computer program code for calculating a score based on the determined amounts of the at least three biomarkers and for carrying out a comparison of the calculated score and the reference score, wherein said evaluation unit further comprises a data base comprising said reference score, whereby it is differentiated between heart failure and pulmonary disease.

The terms “score” and “reference score” are determined elsewhere herein.

In an embodiment, the device further comprises at least one further analysing unit comprising at least one detector for NT-proBNP and/or BNP, wherein said further analyzing unit is adapted for determining the amounts BNP and/or NT-proBNP detected by the at least one detector.

Thus, the present invention relates to a diagnostic device comprising:
a) an analysing unit comprising at least one detector for the at least one biomarker, in particular for the at least three biomarkers as referred to herein in connection with the present invention, wherein said analyzing unit is adapted for determining the amount(s) of the said biomarker(s) detected by the at least one detector, and, optionally, at least one further analysing unit comprising at least one detector for NT-proBNP and/or BNP, wherein said further analyzing unit is adapted for determining the amounts BNP and/or NT-proBNP detected by the at least one detector for NT-proBNP and/or BNP, and operatively linked thereto;
b) an evaluation unit comprising a computer comprising tangibly embedded a computer program code for carrying out a comparison of the determined amount(s) of the least one or the at least three biomarkers and, optionally, of BNP or NT-proBNP, and the reference amounts and a data base comprising said reference amounts for the said biomarkers, whereby it is differentiated between heart failure and pulmonary disease.

Alternatively, the evaluation unit under b) comprises a computer comprising tangibly embedded a computer program code for calculating a score based on the determined amounts of the at least three biomarkers and of BNP and/or NT-proBNP, and for carrying out a comparison of the calculated score and the reference score, wherein said evaluation unit further comprises a data base comprising said reference score, whereby it is differentiated between heart failure and pulmonary disease.

Preferably, the devices are adapted to carry out the method of the present invention.

Preferably, the computer program code is capable of executing steps of the method of the present invention as specified elsewhere herein in detail. Accordingly, the device can be used for differentiating in a subject between heart failure and pulmonary disease as specified herein based on a sample of a subject.

In a preferred embodiment, the device comprises a further database comprising the kind of regulation and/or fold of regulation values indicated for the respective biomarkers in Table land a further tangibly embedded computer program code for carrying out a comparison between the determined kind of regulation and/or fold of regulation values and those comprised by the database. In another preferred embodiment, the device comprises a further database comprising the kind of regulation and/or fold of regulation values indicated for the score(s) calculated based on the amounts of the at least three biomarkers, and a further tangibly embedded computer program code for carrying out a comparison between the determined kind of regulation and/or fold of regulation values for the score and those comprised by the database.

In general, the present invention contemplates the use of at least three biomarkers as referred to herein in connection with the method of differentiating in a subject between heart failure and pulmonary disease, and optionally of BNP or NT-proBNP, in a sample of a subject for differentiating in a subject between heart failure and pulmonary disease or for the preparation of a pharmaceutical and/or diagnostic composition for differentiating in a subject between heart failure and pulmonary disease. Preferred combinations of biomarkers are disclosed elsewhere herein.

In general, the present invention contemplates the use of at least one biomarker selected from the biomarkers shown in column 1 of table 1, and optionally of BNP or NT-proBNP, in a sample of a subject for differentiating in a subject between heart failure and pulmonary disease or for the preparation of a pharmaceutical and/or diagnostic composition for differentiating in a subject between heart failure and pulmonary disease.

The present invention also relates to a kit for carrying out the method of the present invention, said kit comprising detection agents for each of the biomarkers of the at least three biomarkers as set forth in connection with the method of differentiating in a subject between heart failure and pulmonary disease. In an embodiment, the kit may further comprise a detection agent for a natriuretic peptide such as NT-proBNP or BNP.

The term “kit” as used herein refers to a collection of the aforementioned components, preferably, provided separately or within a single container. The detection agents may be provided in the kit of the invention in a “ready-to-use” liquid form or in dry form. The kit may further include controls, buffers, and/or reagents. The kit also comprises instructions for carrying out the method of the present invention, as well as information on the reference values. These instructions may be in the form of a manual or may be electronically accessible information. The latter information may be provided on a data storage medium or device such as an optical storage medium (e.g., a Compact Disc) or directly on a computer or data processing device.

Suitable detection agents for the biomarkers have been specified elsewhere herein in detail. For example, the detection agents may be antibodies or aptameres or other molecules which are capable of binding to the biomarkers specifically.

The kit of the invention can be, preferably, used for carrying out the method of the present invention, i.e. for differentiating in a subject between heart failure and pulmonary disease as specified elsewhere herein in detail.

The definitions and explanations given herein above also apply to the following method and use.

Moreover, the present invention relates to a method for differentiating between heart failure and pulmonary disease in a subject comprising the steps of:
a) determining in a sample of a subject the amount at least one biomarker selected from the group consisting of phosphatidylcholin (C18:1 C18.1), SM(d18:1/18:1), SM(d18:2/17:0), OSS2, PPO1, PPP, SOP2, SPP1, SSP2 and SSS; and
b) comparing the amounts of the said biomarkers as determined in step a) to a reference, whereby it is differentiated in a subject between heart failure and pulmonary disease.

Moreover, the present invention pertains to the use of at least one biomarker selected from the group consisting of phosphatidylcholin (C18:1 C18.1), SM(d18:1/18:1), SM(d18:2/17:0), OSS2, PPO1, PPP, SOP2, SPP1, SSP2 and SSS in a sample of a subject for differentiating between heart failure and pulmonary disease.

### Embodiments/Items of the Present Invention

Items for Panel 1

In a preferred embodiment, the amounts of the biomarkers in panel 1 are determined. The heart failure to be diagnosed may be classified as NYHA class I or II. Preferably, the determination is carried out for the diagnosis of HFrEF, in particular for the diagnosis of HFrEFwith a left ventricular ejection fraction of lower than 50% but larger than 35%. Further, it is envisaged that the subject does not show symptoms of heart failure.

In addition, it is envisaged to determine the amount of NT-proBNP in addition of the at least three biomarkers. Preferably, a correction for confounders (in particular age, BMI and gender) is not carried out.

With respect to panel 1, the following items are preferred. The definitions and explanations, given in the specification above, preferably, apply mutatis mutandis to following preferred items of the present invention.
1. A method for differentiating in a subject between heart failure and pulmonary disease comprising the steps of:
   a. determining in a sample of a subject the amounts of the SM23, OSS2 and PC4, and
   b. comparing the amounts as determined in step a. to a reference, whereby it is differentiated in a subject between heart failure and pulmonary disease
2. The method of item 1, wherein the subject is suspected to suffer from heart failure or pulmonary disease.
3. The method of item 1 and 2, wherein the subject suffers from shortness of breath.
4. The method of any one of items 1 to 3, wherein the subject is a human subject.
5. The method of any one of items to 1 to 4, wherein the sample is blood, serum or plasma.
6. The method of any one of items 1 to 5, wherein the method does not comprise the determination of NT-proBNP or BNP.
7. The method of any one of items 1 to 6, further comprising determining the amount of NT-proBNP or BNP in a sample/the sample from the subject and comparing the amount of NT-proBNP or BNP to a reference.
8. The method of any one of items 1 to 7, further comprising carrying out a correction for confounders.
9. The method of item 8, wherein the confounders are age, BMI and/or gender, in particular age, BMI and gender.
10. The method of item 1 to 9, wherein no correction for confounders is carried out.
11. The method of item 10, wherein no correction for confounders age, BMI and gender is carried out.
12. The method of any one of items 1 to 11, wherein in step b) a score is calculated based on the determined amounts of the at least three biomarkers, wherein the reference is a reference score, and wherein the score is compared to the reference score.
13. The method of any one of items 1 to 12, wherein the reference or the reference score is from a subject or group of subjects known to suffer from pulmonary disease.
14. The method of item 12, wherein a value for each of the at least three biomarkers, or a score in the test sample being essentially identical as compared to the reference or reference score is indicative for the presence of pulmonary disease.
15. The method of any one of items 1 to 12, wherein the reference or the reference score is from a subject or group of subjects known to suffer from heart failure.
16. The method of any one of item 15, wherein a value for each of the at least three biomarkers found, or a score in the test sample being essentially identical as compared to the reference or reference score is indicative for the presence of the heart failure.
17. The method of any one of items 1 to 16, wherein the amounts of the at least three biomarkers are determined by mass spectrometry (MS).
18. The method of item 17, wherein the mass spectrometry is LC-MS, in particular LC-MS/MS, or HPLC-MS, in particular HPLC-MS/MS.
19. The method of items 17 and 18, wherein the mass spectrometry comprises an ionization step in which the at least three biomarkers are ionized.
20. The method of item 19, wherein the ionization step is carried out by electrospray ionization, in particular by positive ion mode electrospray ionization.
21. The method of items 1 to 20, wherein OSS2 is TAG(C18:1, C18:0, C18:0).
22. The method of items 1 to 21, wherein Phosphatidylcholine (C16:0 018:2).
23. The method of any one of items 1 to 22, wherein SM23 is Sphingomyelin(d18:1/23:1), ii) Sphingomyelin(d18:2/23:0), iii) Sphingomyelin(d17:1/24:1), or iv) a combination of Sphingomyelin(d18:1/23:1), Sphingomyelin(d18:2/23:0) and Sphingomyelin(d17:1/24:1).
24. The method of any one of items 23, wherein the amount of SM23 is determined by i) determining the amount of the amount of Sphingomyelin(d18:1/23:1), the amount of Sphingomyelin (d17:1/24:1), or, in particular, the combined amount of Sphingomyelin(d18:1/23:1), Sphingomyelin(d18:2/23:0), and Sphingomyelin(d17:1/24:1). 1

25. The method of any one of items 1 to 24, wherein the amount of SM23 is determined by determining the combined amount of Sphingomyelin(d18:1/23:1), Sphingomyelin(d18:2/23:0), and Sphingomyelin(d17:1/24:1).
26. A diagnostic device for carrying out the method according to any one of items 1 to 25, comprising:
   a) an analysing unit comprising at least one detector for the biomarkers SM23, OSS2 and PC4, wherein said analyzing unit is adapted for determining the amounts of the said biomarkers detected by the at least one detector, and, operatively linked thereto;
   b) an evaluation unit comprising a computer comprising tangibly embedded a computer program code for carrying out a comparison of the determined amounts of the biomarkers, and reference amounts and a data base comprising said reference amounts for the said biomarkers, whereby it is differentiated between pulmonary disease and heart failure. 1

27. Use of SM23, OSS2 and PC4 in a sample of a subject for differentiating in a subject between heart failure and pulmonary disease or for the preparation of a pharmaceutical and/or diagnostic composition for differentiating in a subject between heart failure and pulmonary disease.

### THE FIGURE SHOWS

FIG. 1: Prediction probabilities for the 131 HFrEF patients from the testing set (crosses) and the 34 patients with pulmonary disease (circles) calculated on single patient level using Panel 1+NT-proBNP in comparison to NT-proBNP alone.

The following Examples shall illustrate the invention. They shall, however, not be construed as limiting the scope of the invention.

### EXAMPLE 1

### Biomarkers and Biomarker Panels for the Differentiation of Heart Failure Versus Pulmonary Disease

Biomarkers for a differentiation of heart failure versus pulmonary disease are shown in Table 1. Biomarker panels for the differentiation of heart failure versus pulmonary disease representing combinations of the biomarkers from Table 1 are shown in Tables 2 and 2A. Table 1 also lists the direction of change of the biomarkers when comparing heart failure patients against healthy controls. The direction of change was calculated for each biomarker by ANOVA using the ANOVA model log₁₀(biomarker)˜group+center+(gender+age+BMI)², where ‘biomarker’ is the peak area of the biomarker divided by the peak area of the respective internal standard (see Example 3), group is the study group to which a particular sample belongs (heart failure patient or healthy control), center is the clinical center where the sample was taken (Berlin, Kiel, or Heidelberg), gender, age, and BMI are the gender, age, and BMI, respectively, of the subject from which the sample was taken.

For the biomarkers from Table 1, the direction of change in HFrEF patients relative to subjects with pulmonary disease was calculated by ANOVA using the ANOVA model log₁₀(biomarker)˜group+center+(gender+age+BMI)², where ‘biomarker’ was the absolute concentration of the biomarker given in μg/dl (see Example 3, below), ‘group’ was the study group to which a particular sample belonged (HFrEF patient or patient with pulmonary disease), ‘center’ was the clinical center where the sample was taken (Berlin, Kiel, or Heidelberg), gender, age, and BMI were the gender, age, and BMI, respectively, of the subject from which the sample was taken

Biomarker panels which represent specific combinations of the biomarkers from Table 1 for the differentiation of HF versus pulmonary diseases are shown in Table 2 and Table 2A. The last column of Table 1 shows the comparison of the amounts of the individual biomarkers in heart failure patients as compared to patients with pulmonary disease.

TABLE 1
Biomarkers used for panel composition, their analytical characteristics, and their
direction of change when comparing heart failure patients to healthy controls.

| | | | | | | | Direction | |
|---|---|---|---|---|---|---|---|---|
| | | | | | Transition | | of change: | Direction |
| | | | | | (parent/ | | heart failure | of change: |
| | | | | | fragment) | Retention | vs. healthy | heart failure |
| Biomarker | Analyte 1 | Analyte 2 | Analyte 3 | Group | (Da) | time (min) | controls | vs. dyspnea |
| Cer(d16:1/24:0) | Cer(d16:1/24:0) | | | CER | 622.6/236.2 | 2.83 | Down | No change |
| Cer(d17:1/24:0) | Cer(d17:1/24:0) | | | CER | 636.6/250.2 | 2.95 | Down | No change |
| Cer(d18:1/23:0) | Cer(d18:1/23:0) | | | CER | 636.5/264.2 | 2.95 | Down | No change |
| Cer(d18:1/24:1) | Cer(d18:1/24:1) | | | CER | 648.6/264.2 | 2.83 | Down | No change |
| Cer(d18:2/24:0) | Cer(d18:2/24:0) | | | CER | 648.6/262.2 | 2.90 | Down | No change |
| CE C18:0 | CE C18:0 | | | CE | 670.7/369.3 | 4.72 | Down | No change |
| CE C18:2 | CE C18:2 | | | CE | 666.7/369.3 | 4.47 | Down | No change |
| Glutamic acid | Glutamic acid | | | AA | 148.0/84.1 | 0.23 | Down | No change |
| PC4 | PC (C16:0 C18:2) | | | PC | 758.6/184.1 | 1.72 | Down | No change |
| PC8 | PC (C18:0 C18:2) | PC (C18:1 C18:1) | | PC | 786.6/184.1 | 1.88 | Down | No change |
| SM10 | SM(d18:1/18:0) | SM(d16:1/20:0) | | SM | 731.5/184.1 | 1.70 | Down | No change |
| SM18 | SM(d18:1/21:0) | SM(d16:1/23:0) | SM(d17:1/22:0) | SM | 773.5/184.1 | 1.97 | Down | No change |
| SM2 | SM(d18:1/14:0) | SM(d16:1/16:0) | | SM | 675.5/184.1 | 1.47 | Down | No change |
| SM21 | SM(d17:1/23:0) | SM(d18:1/22:0) | SM(d16:1/24:0) | SM | 787.5/184.1 | 2.05 | Down | Down |
| SM23 | SM(d18:1/23:1) | SM(d18:2/23:0) | SM(d17:1/24:1) | SM | 799.5/184.1 | 2.02 | Down | No change |
| SM24 | SM(d18:1/23:0) | SM(d17:1/24:0) | | SM | 801.5/184.1 | 2.16 | Down | No change |
| SM28 | SM(d18:1/24:0) | | | SM | 815.5/184.1 | 2.27 | Down | Down |
| SM29 | SM(d18:2/17:0) | | | SM | 715.5/184.1 | 1.57 | Down | No change |
| SM3 | SM(d17:1/16:0) | | | SM | 689.5/184.1 | 1.52 | Down | Down |
| SM5 | SM(d18:1/16:0) | SM(d16:1/18:0) | | SM | 703.5/184.1 | 1.57 | Down | No change |
| SM8 | SM(d18:2/18:1) | | | SM | 727.5/184.1 | 1.55 | Down | No change |
| SM9 | SM(d18:1/18:1) | SM(d18:2/18:0) | | SM | 729.5/184.1 | 1.62 | Down | No change |
| OSS2 | TAG C18:1 C18:0 C18:0 | | | TAG | 906.9/605.4 | 4.87 | Up | No change |
| PPO1 | TAG C16:0 C16:0 C18:1 | | | TAG | 850.8/577.5 | 4.64 | Up | No change |
| PPP | TAG C16:0 C16:0 C16:0 | | | TAG | 824.8/551.5 | 4.61 | Up | No change |
| SOP2 | TAG C18:0 C18:1 C16:0 | | | TAG | 878.9/577.6 | 4.76 | Up | No change |
| SPP1 | TAG C18:0 C16:0 C16:0 | | | TAG | 852.9/579.6 | 4.74 | Up | No change |
| SSP2 | TAG C18:0 C18:0 C16:0 | | | TAG | 880.9/579.6 | 4.85 | Up | No change |
| SSS | TAG C18:0 C18:0 C18:0 | | | TAG | 908.9/607.6 | 4.96 | Up | No change |

AA = amino acid, CE = cholesteryl ester, CER = ceramide, PC = phosphatidylcholine, SM = sphingomyelin, TAG = triacylglycerides.

TABLE 2
Biomarker panels respresenting specific combinations of the biomarkers from
Table 1 for the differentiation of heart failure versus pulmonary disease

| Panel | | No. of |
|---|---|---|
| Number | Panel Composition (Biomarkers) | Biomarkers |
| 1 | SM23; OSS2; PC4 | 3 |
| 2 | OSS2; SM23; CE C18:2 | 3 |
| 3 | SOP2; OSS2; PC4; CE C18:2; SM18; SM28; SM24; SSP2; SM23 | 9 |
| 4 | OSS2; CE C18:2; SM23 | 3 |
| 5 | SOP2; OSS2; SM18; CE C18:2; SM24; SM28; Cer(d16:1/24:0); PC4 | 8 |
| 6 | SM18; OSS2; CE C18:2 | 3 |
| 7 | SM23; SOP2; CE C18:2 | 3 |
| 8 | SM23; SOP2; CE C18:2 | 3 |
| 9 | SOP2; SM24; SSP2; SM23; CE C18:2; SM28; SM18; PC4 | 8 |
| 10 | SM23; SOP2; SM28 | 3 |
| 11 | SM24; SOP2; CE C18:2; SM28; SSP2; SM18; OSS2; SM2; Cer(d16:1/24:0) | 9 |
| 12 | SM24; SOP2; CE C18:2 | 3 |
| 13 | OSS2; PC4; SM23 | 3 |
| 14 | OSS2; CE C18:2; SM3 | 3 |
| 15 | SM18; SM28; PC4; OSS2; SOP2; CE C18:2; PPO1; SM10 | 8 |
| 16 | CE C18:2; SOP2; SM18 | 3 |
| 17 | SM18; OSS2; SOP2; CE C18:2; PC4; SPP1; SM24; Cer(d16:1/24:0); SM28; SM21 | 10 |
| 18 | SM18; CE C18:2; OSS2 | 3 |
| 19 | SM23; SOP2; CE C18:2 | 3 |
| 20 | OSS2; SM23; CE C18:2 | 3 |
| 21 | CE C18:2; OSS2; SM23; SM24; SSP2 | 5 |
| 22 | OSS2; SM23; CE C18:2 | 3 |
| 23 | SM23; CE C18:2; OSS2; SSP2; SM24 | 5 |
| 24 | SM24; SSP2; OSS2 | 3 |
| 25 | SOP2; PC4; SM3 | 3 |
| 26 | OSS2; PC4; SM3 | 3 |
| 27 | SSP2; PPO1; SM18; CE C18:2; OSS2; SOP2; PC4; SM28 | 8 |
| 28 | SOP2; SM28; PC4 | 3 |
| 29 | Cer(d16:1/24:0); SM28; PC4; SM24; CE C18:2; SPP1; OSS2; SOP2 | 8 |
| 30 | OSS2; CE C18:2; SM24 | 3 |
| 31 | SOP2; SM23; PC4 | 3 |
| 32 | OSS2; CE C18:2; SM23 | 3 |
| 33 | SM24; SOP2; OSS2; CE C18:2; SSP2; PC4; SM28 | 7 |
| 34 | SOP2; SM24; PC4 | 3 |
| 35 | OSS2; CE C18:2; SOP2; SM18; SM24; SSP2; SM28; PC4 | 8 |
| 36 | OSS2; SM24; CE C18:2 | 3 |
| 37 | SM5; PPO1; SM23 | 3 |
| 38 | SM5; SOP2; SM24 | 3 |
| 39 | SSP2; CE C18:2; SM18; SM23; Cer(d16:1/24:0); PC4; SOP2; PPO1; SM28; SM24 | 10 |
| 40 | SM24; SOP2; PC4 | 3 |
| 41 | PC4; SM5; CE C18:2; SM2; PPO1; SOP2; SM28; Cer(d16:1/24:0); SM24 | 9 |
| 42 | SM24; CE C18:2; SOP2 | 3 |
| 43 | Cer(d18:1/24:1); SOP2; CE C18:2; SM18 | 4 |
| 44 | SM18; CE C18:2; SOP2 | 3 |
| 45 | SOP2; SM24; CE C18:2; SM18 | 4 |
| 46 | SM18; CE C18:2; SOP2 | 3 |
| 47 | SSP2; PC4; Cer(d18:1/24:1); SM28; SM24; SOP2; CE C18:2; SM18 | 8 |
| 48 | SM18; CE C18:2; SOP2 | 3 |
| 49 | SM28; CE C18:2; SOP2; SM18 | 4 |
| 50 | SM18; CE C18:2; SOP2 | 3 |
| 51 | SM23; CE C18:2; SOP2 | 3 |
| 52 | SM23; CE C18:2; SOP2 | 3 |
| 53 | SOP2; SM24; CE C18:2; SM18; PC4; SM28; PPO1; Cer(d16:1/24:0); OSS2; Cer(d18:1/24:1) | 10 |
| 54 | SM18; CE C18:2; SOP2 | 3 |
| 55 | SOP2; SM18; SM24; CE C18:2; Cer(d16:1/24:0); SM28; OSS2; PC4 | 8 |
| 56 | SM18; CE C18:2; SOP2 | 3 |
| 57 | SOP2; SM23; PC4 | 3 |
| 58 | SOP2; SSP2; SM5; SPP1; SM2; SM3; PC4; CE C18:2; Glutamic acid | 9 |
| 59 | SOP2; SM5; SM2 | 3 |
| 60 | SOP2; SM23; SSP2; SM24; CE C18:2; PC4; OSS2 | 7 |
| 61 | SOP2; SM23; CE C18:2 | 3 |
| 62 | SOP2; SSP2; SM2; CE C18:2; SM24; OSS2; SM18; PC4; SM28 | 9 |
| 63 | SOP2; SM18; CE C18:2 | 3 |
| 64 | SOP2; PC4; OSS2; Cer(d18:1/24:1); SM24; PC8; SM21; Cer(d17:1/24:0) | 8 |
| 65 | SOP2; PC4; SM24 | 3 |
| 66 | SOP2; SM18; PC4; SM21 | 4 |
| 67 | SOP2; SM18; PC4 | 3 |
| 68 | SOP2; PC4; SM28; SM18; Cer(d18:1/24:1); Cer(d18:2/24:0); SM9 | 7 |
| 69 | SOP2; PC4; SM18 | 3 |
| 70 | PC4; SOP2; SM18; SM24; OSS2; SPP1; SM21; Cer(d16:1/24:0); SM28; CE C18:2 | 10 |
| 71 | SM18; SOP2; PC4 | 3 |
| 72 | SOP2; PC4; SM5 | 3 |
| 73 | SOP2; SM3; PC4; SM21 | 4 |
| 74 | SOP2; SM3; PC4 | 3 |
| 75 | SOP2; SM18; PC4; SM28; OSS2; SSP2; CE C18:2 | 7 |
| 76 | SOP2; SM18; PC4 | 3 |
| 77 | SOP2; OSS2; SM18; SM24; PC4; CE C18:2; Cer(d16:1/24:0); SM28 | 8 |
| 78 | OSS2; SM18; CE C18:2 | 3 |
| 79 | SPP1; SOP2; SM5; PC4; SM3; SSP2 | 6 |
| 80 | SPP1; SM5; PC4 | 3 |
| 81 | SPP1; SM3; SM5; SSP2; PC4; Glutamic acid; CE C18:0; OSS2; SOP2 | 9 |
| 82 | SPP1; SM3; SM5 | 3 |
| 83 | SPP1; SOP2; SSP2; PC4; SM23; SM3; SM3; CE C18:2 | 8 |
| 84 | SPP1; SM23; SOP2 | 3 |
| 85 | SM3; SPP1; SSP2; SM5; SOP2; PC4 | 6 |
| 86 | SPP1; SM3; SM5 | 3 |
| 87 | SOP2; Cer(d18:1/24:1); PC8; PPO1; Cer(d18:1/23:0); PC4; SM24; SM28; SM9 | 9 |
| 88 | SOP2; PC4; SM24 | 3 |
| 89 | Cer(d18:1/23:0); SM24; SOP2; Cer(d18:1/24:1); PC4; PPO1; PCS; SSS; SM18 | 9 |
| 90 | SM24; SOP2; PC4 | 3 |
| 91 | PC4; SM28; SM24; Cer(d18:1/24:1); PC8; SOP2; SM9; PPO1; SM21; SSP2 | 10 |
| 92 | PC4; SM28; SOP2 | 3 |
| 93 | SM24; PC4; SM18; SOP2; SM21; SPP1; Cer(d18:1/23:0); SSP2 | 8 |
| 94 | SM24; SM18; SOP2 | 3 |
| 95 | SOP2; PC8; SPP1; PC4; SSP2; PPO1; SM5; SM18; Cer(d17:1/24:0) | 9 |
| 96 | SOP2; PC8; SM5 | 3 |
| 97 | SPP1; PC4; SM3; SOP2; SSP2; Cer(d18:1/23:0); SM24; SM5; Glutamic acid | 9 |
| 98 | SPP1; SM3; PC4 | 3 |
| 99 | SOP2; PC4; SPP1; PC8; SSP2; SM28; SM24; PPO1; SM18; Cer(d17:1/24:0) | 10 |
| 100 | SOP2; PC4; SM24 | 3 |
| 101 | PC4; SPP1; SM24; SSP2; SM3; SOP2; SM18; Glutamic acid; Cer(d17:1/24:0); Cer(d18:1/23:0) | 10 |
| 102 | SPP1; SM18; PC4 | 3 |
| 103 | CE C18:2; SSS; Cer(d17:1/24:0) | 3 |
| 104 | PC4; SOP2; CE C18:2 | 3 |
| 105 | CE C18:2; PC4; SM29; SOP2 | 4 |
| 106 | CE C18:2; PC8; SM29; SSP2 | 4 |
| 107 | CE C18:2; PC4; SM8; SSP2 | 4 |
| 108 | CE C18:2; PC8; SM8; SSP2 | 4 |
| 109 | CE C18:2; PC4; SM29; PPO1 | 4 |
| 110 | CE C18:2; PC8; SM29; PPO1 | 4 |
| 111 | CE C18:2; PC4; SM8; PPO1 | 4 |
| 112 | CE C18:2; PC8; SM8; PPO1 | 4 |
| 113 | CE C18:2; PC4; SM29; PPP | 4 |
| 114 | CE C18:2; PC8; SM29; PPP | 4 |
| 115 | CE C18:2; PC4; SM8; PPP | 4 |
| 116 | CE C18:2; PC8; SM29; SOP2 | 4 |
| 117 | CE C18:2; PC8; SM8; PPP | 4 |
| 118 | CE C18:2; PC4; SM8; SOP2 | 4 |
| 119 | CE C18:2; PC8; SM8; SOP2 | 4 |
| 120 | CE C18:2; PC4; SM29; SPP1 | 4 |
| 121 | CE C18:2; PC8; SM29; SPP1 | 4 |
| 122 | CE C18:2; PC4; SM8; SPP1 | 4 |
| 123 | CE C18:2; PC8; SM8; SPP1 | 4 |
| 124 | CE C18:2; PC4; SM29; SSP2 | 4 |
| 125 | PC4; SOP2; CE C18:2; SM18 | 4 |
| 126 | PC4; SOP2; CE C18:0; SM18 | 4 |
| 127 | PC4; SOP2; CE C18:2; SM21 | 4 |
| 128 | PC4; SOP2; CE C18:0; SM21 | 4 |
| 129 | PC4; SOP2; CE C18:2; SM23 | 4 |
| 130 | PC4; SOP2; CE C18:0; SM23 | 4 |
| 131 | PC4; SOP2; CE C18:2; SM24 | 4 |
| 132 | PC4; SOP2; CE C18:0; SM24 | 4 |
| 133 | SOP2; CE C18:0; SM18; PC4; SPP1 | 5 |
| 134 | SOP2; CE C18:2; SM21; PC4; SPP1 | 5 |
| 135 | SOP2; CE C18:0; SM21; PC4; SPP1 | 5 |
| 136 | SOP2; CE C18:2; SM23; PC4; SPP1 | 5 |
| 137 | SOP2; CE C18:0; SM23; PC4; SPP1 | 5 |
| 138 | SOP2; CE C18:2; SM24; PC4; SPP1 | 5 |
| 139 | SOP2; CE C18:0; SM24; PC4; SPP1 | 5 |
| 140 | SOP2; CE C18:2; SM18; PC4; SSP2 | 5 |
| 141 | SOP2; CE C18:0; SM18; PC4; SSP2 | 5 |
| 142 | SOP2; CE C18:2; SM21; PC4; SSP2 | 5 |
| 143 | SOP2; CE C18:0; SM21; PC4; SSP2 | 5 |
| 144 | SOP2; CE C18:2; SM23; PC4; SSP2 | 5 |
| 145 | SOP2; CE C18:0; SM23; PC4; SSP2 | 5 |
| 146 | SOP2; CE C18:2; SM24; PC4; SSP2 | 5 |
| 147 | SOP2; CE C18:0; SM24; PC4; SSP2 | 5 |
| 148 | SOP2; CE C18:2; SM18; PC4; PPO1 | 5 |
| 149 | SOP2; CE C18:0; SM18; PC4; PPO1 | 5 |
| 150 | SOP2; CE C18:2; SM21; PC4; PPO1 | 5 |
| 151 | SOP2; CE C18:0; SM21; PC4; PPO1 | 5 |
| 152 | SOP2; CE C18:2; SM23; PC4; PPO1 | 5 |
| 153 | SOP2; CE C18:0; SM23; PC4; PPO1 | 5 |
| 154 | SOP2; CE C18:2; SM24; PC4; PPO1 | 5 |
| 155 | SOP2; CE C18:0; SM24; PC4; PPO1 | 5 |
| 156 | SOP2; CE C18:2; SM18; PC4; PPP | 5 |
| 157 | SOP2; CE C18:0; SM18; PC4; PPP | 5 |
| 158 | SOP2; CE C18:2; SM21; PC4; PPP | 5 |
| 159 | SOP2; CE C18:0; SM21; PC4; PPP | 5 |
| 160 | SOP2; CE C18:2; SM23; PC4; PPP | 5 |
| 161 | SOP2; CE C18:0; SM23; PC4; PPP | 5 |
| 162 | SOP2; CE C18:2; SM24; PC4; PPP | 5 |
| 163 | SOP2; CE C18:0; SM24; PC4; PPP | 5 |
| 164 | SOP2; CE C18:2; SM18; PC4; SPP1 | 5 |
| 165 | PC4; PPP; CE C18:0; SM18 | 4 |
| 166 | PC4; SPP1; CE C18:2; SM21 | 4 |
| 167 | PC4; SSP2; CE C18:2; SM21 | 4 |
| 168 | PC4; PPO1; CE C18:2; SM21 | 4 |
| 169 | PC4; PPP; CE C18:2; SM21 | 4 |
| 170 | PC4; SPP1; CE C18:0; SM21 | 4 |
| 171 | PC4; SSP2; CE C18:0; SM21 | 4 |
| 172 | PC4; PPO1; CE C18:0; SM21 | 4 |
| 173 | PC4; SPP1; CE C18:2; SM18 | 4 |
| 174 | PC4; PPP; CE C18:0; SM21 | 4 |
| 175 | PC4; SPP1; CE C18:2; SM23 | 4 |
| 176 | PC4; SSP2; CE C18:2; SM23 | 4 |
| 177 | PC4; PPO1; CE C18:2; SM23 | 4 |
| 178 | PC4; PPP; CE C18:2; SM23 | 4 |
| 179 | PC4; SPP1; CE C18:0; SM23 | 4 |
| 180 | PC4; SSP2; CE C18:0; SM23 | 4 |
| 181 | PC4; PPO1; CE C18:0; SM23 | 4 |
| 182 | PC4; SSP2; CE C18:2; SM18 | 4 |
| 183 | PC4; PPP; CE C18:0; SM23 | 4 |
| 184 | PC4; SPP1; CE C18:2; SM24 | 4 |
| 185 | PC4; SSP2; CE C18:2; SM24 | 4 |
| 186 | PC4; PPO1; CE C18:2; SM24 | 4 |
| 187 | PC4; PPP; CE C18:2; SM24 | 4 |
| 188 | PC4; SPP1; CE C18:0; SM24 | 4 |
| 189 | PC4; SSP2; CE C18:0; SM24 | 4 |
| 190 | PC4; PPO1; CE C18:0; SM24 | 4 |
| 191 | PC4; PPO1; CE C18:2; SM18 | 4 |
| 192 | PC4; PPP; CE C18:0; SM24 | 4 |
| 193 | PC4; PPP; CE C18:2; SM18 | 4 |
| 194 | PC4; SPP1; CE C18:0; SM18 | 4 |
| 195 | PC4; SSP2; CE C18:0; SM18 | 4 |
| 196 | PC4; PPO1; CE C18:0; SM18 | 4 |
| 197 | PC4; SOP2; CE C18:2; SM28 | 4 |
| 198 | PC4; SOP2; CE C18:0; SM28 | 4 |
| 199 | SM18; SM24; SM28 | 3 |

TABLE 2A
Further biomarker panels representing specific combinations
of the biomarkers from Table 1 for the differentiation
of heart failure versus pulmonary disease

| Panel | | No. of |
|---|---|---|
| Number | Panel Composition (Biomarkers) | Biomarkers |
| 200 | OSS2; SM23; CE C18:2; PC4 | 4 |
| 201 | CE C18:2; SM18; SSS | 3 |
| 202 | CE C18:2; PC8; SM18; SM2; SM24; SSS | 6 |
| 203 | CE C18:2; SM18; SM21; SM24; SSS | 5 |
| 204 | SM24; CE C18:2; SM2 | 3 |
| 205 | SM2; SSS; CE C18:2 | 3 |
| 206 | SM24; SSS; CE C18:2 | 3 |

### EXAMPLE 2

### Study Design—Selection Criteria and Resulting Patient Cohorts

General selection criteria for heart failure patients:

General inclusion criteria were informed written consent, age 35-75 years, BMI 20-35 kg/m², stable weight (±2.0kg) and medication (concerning cardiac, blood pressure, diabetes, lipid, psychopharmacological and thyroid medication) for at least 4 weeks prior to inclusion. General exclusion criteria were NYHA functional class IV, imaging (echocardiography or MRI) >6 months (in exceptional cases <12 months), anemia at the time of sampling (Hb<10 g/dl), ICD shock within 7 days, pregnancy, stroke or myocardial infarction within the last 4 months, surgery or any modification of pharmacological therapy within the last month, known chronic or acute inflammatory disease (e.g. hepatitis B or C, HIV etc., indicated by physical examination and history), reduced renal function, uncontrolled diabetes mellitus (HbA1c>8%), autoimmune diseases (e.g. type 1 diabetes mellitus), malignant tumors within the last 5 years (exception skin basalioma) and relevant organic dysfunction (with exception of HF).

Specific selection criteria for heart failure patients:

Ischemic cardiomyopathy (ICMP) was defined as reduced left ventricular (LV) function with an ejection fraction (EF)≤50% derived from echocardiography or MRI as well as angiographically confirmed history of coronary artery disease (CAD; luminal obstruction>50% of at least one coronary artery), leading to imaging evidence of regional or global wall motion abnormalities. In rare cases without coronary angiography (n=3), alternative imaging evidence for ischemic origin by cardiac magnetic resonance imaging (cMRI) and/or CT-angiography was accepted as inclusion criterion. Dilated cardiomyopathy (DCMP) was defined as reduced LV function with LVEF≤50% either on echocardiography or MRI. Exclusion criteria was angiographically confirmed history of coronary artery disease (CAD; luminal obstruction>50% of at least one coronary artery), leading to imaging evidence of regional wall motion abnormalities. In rare cases with evidence of CAD in coronary angiography (n=5) or missing coronary angiography (n=9), patients were classified as DCMP, when there was evidence from myocardial biopsy and/or imaging that the extent of reduced LV function is not explained by CAD or ischemic origin.

Hypertrophic cardiomyopathy (HCMP) was defined as concentric heart hypertrophy (echocardiography: cardiac septum>12 mm and posterior myocardial wall>11 mm) and with a diastolic heart failure (non or mildly impaired pump function with LVEF of ≥50%) without a cardiac septum thickness>18 mm.

Selection criteria for healthy control group:

Exclusion criteria for normal control subjects were age<35 or age>75 years, any chronic disease or acute illness, any intake of regular medication, active smoking, chronic drug or alcohol abuse (by history or clinical evidence). All controls had a normal physical examination, a normal resting 12-lead electrocardiogram, no hypertensive blood pressure values in serial measurements. Echocardiography or cardiac MRI (cMRI) revealed absence of LV hypertrophy (norm: septal wall thickness<11 mm, posterior wall thickness<11 mm), LV dilatation (norm≤55 mm), LV systolic dysfunction (norm: LVEF≥55%), valvular heart disease, or stress-induced LV wall motion abnormalities or perfusion defects on stress echocardiography or cMRI.

Selection criteria for patients with pulmonary diseases:

Inclusion criteria were dyspnea due to pulmonary origin including chronic obstructive pulmonary disease (COPD), pulmonary fibrosis or asthma (with FEV1/FVC<0.70; FEV1≥30%). Also patients were required to have an echocardiographic examination to confirm an LVEF≥55%. Exclusion criteria were COPD GOLD stage IV (FEV1/FVC<0.70; FEV1<30%) or chronic respiratory failure, cystic fibrosis, acute exacerbation and pulmonary hypertension

Heart failure patient cohort:

The heart failure patient cohort resulting from the criteria described above included 823 subjects comprising 190 male and female DCMP patients, 181 male and female ICMP patients, and 208 male and female HCMP patients as well as 244 healthy controls.

From this heart failure patient cohort, 534 randomly selected subjects (stratified by subgroup) were taken as training set for the statistical analysis, and the remaining 289 subjects were taken as testing set. Of the 534 randomly selected subjects in the training set, 123 were DCMP patients, 117 were ICMP patients, 134 were HCMP patient, and 160 were healthy controls. Of the 289 subjects in the testing set, 67 were DCMP patients, 64 were ICMP patients, 74 were HCMP patients, and 84 were healthy controls.

Pulmonary disease patient cohort:

The respective pulmonary disease cohort included 34 subjects.

### EXAMPLE 3

### Analytical Protocol

The samples from heart failure patients were analyzed semi-quantitatively, i.e. the amounts of the biomarkers were obtained as the peak area of each biomarker divided by the peak area of the respective internal standard in the same sample. PC 5-40 was used as internal standard for SM23, CE C18:2, and OSS2, whereas PC 5-70 was used as internal standard for PC4. Both PC 5-40 and PC 5-70 refer to the same chemical substance (phosphatidylcholine [PC] C19:0/C19:0), but differ in the MRM settings that were used for data acquisition (Table 4). The analysis of the samples from heart failure patients comprised the steps ‘Sample extraction and HPLC-MS/MS system’, ‘Liquid chromatography’, and ‘Mass spectrometry using multiple-reaction-monitoring (MRM)’, which are described below.

The samples from patients with pulmonary disease were analyzed quantitatively, i.e. the amounts of the biomarkers were obtained as absolute concentrations and were reported in the unit μg/dl. Absolute quantification was achieved using calibration samples containing known amounts of SM27 (Sphingomyelin d18:1124:1, for the quantification of SM23, purchased from Avanti Polar Lipids, CA, U.S.A.), PC4, CE C18:2, and OSS2. Absolute concentration were calculated by comparing the ratio of the peak area of each biomarker to the peak area of the respective internal standard in each sample from patients with pulmonary disease to the ration of the peak area of each biomarker to the peak area of the respective internal standard in the calibration samples. PC 5-40 was used as internal standard for SM23, CE C18:2, and OSS2, whereas PC 5-70 was used as internal standard for PC4. Both PC 5-40 and PC 5-70 refer to the same chemical substance (phosphatidylcholine [PC] C19:0/C19:0), but differ in the MRM settings that were used for data acquisition (Table 4). The analysis of the samples from patients with pulmonary disease comprised the steps ‘Sample extraction and HPLC-MS/MS system’, ‘Preparation of calibration samples’, ‘Liquid chromatography’, and ‘Mass spectrometry using multiple-reaction-monitoring (MRM)’, which are described below.

Sample extraction and HPLC-MS/MS system:

10 μl plasma were mixed with 1500 μl extraction solvent containing methanol/dichloromethane (in a ratio of 2:1, v/v) and 10 μl internal standard mixture in a 2 ml safelock microcentrifuge tube (Eppendorf, Germany). The internal standard solution contained 41.28 μg/ml phosphatidylcholine (PC) C19:0/C19:0 (Avanti Polar Lipids, CA, U.S.A.) in extraction solvent. Ultrapure water (Milli-Q water system, Millipore) and analytical grade chemicals were used for extraction, dilution or as LC solvents. Quality control and reference sample were prepared from commercially available human plasma (RECIPE Chemicals +Instruments GmbH). Delipidized plasma (Plasma, Human, Defibrinated, Delipidized, 2× Charcoal treated, Highly Purified; USBio) was used for the preparation of calibrators and blanks. After thoroughly mixing at 20° C. for 5min, the precipitated proteins were removed by centrifugation for 10 min. An aliquot of the liquid supernatant was transferred to an appropriate glass vial and stored at −20° C. until analysis by LC-MS/MS. Up to 5 μL of the crude extract were injected into an HPLC-MS/MS system consisting of an Agilent 1100 LC system (Agilent Technologies, Waldbronn, Germany) coupled to an ABSciex™ API 4000 triple quadrupole mass spectrometer (AB-SCIEX, Toronto, Canada; the method is intended to be compatible with e.g. the ABSciex™ 3200MD benchtop LC-MS/MS system).

Preparation of calibration samples:

In order to generate calibration curves for the absolute quantification of SM23, PC4, CE C18:2, and OSS2, 7 calibration solutions (STD1, STD2, STD3, STD4, STDS, STDG, STD7) comprising known amounts of SM27 (for the quantification of SM23), PC4, CE C18:2, and OSS2 were prepared in MeOH/CH₂Cl₂ (2/1, v/v) (Table 5). Concentration ratios (relative to CE C18:2) of the compounds within the calibration samples/calibration solutions for calibration sample preparation are given in Table 5a. From each of these calibration solutions, 50 μl were added to a mixture of 10 μl delipidized plasma (commercially available) and 750 μl MeOH/CH2Cl2 (2/1). 10 μl internal standard were added, as described above, and further 700 μl MeOH/CH2Cl2 (2/1) were added in order to achieve a final calibration sample volume of 1520 μl. The resulting 7 calibration samples were then treated the same way as the actual plasma samples for biomarker determination.

Liquid chromatography:

HPLC analysis was performed at 55° C. on commercially available reversed phase separation columns with C18 stationary phases (Ascentis® Express C18 column (5 cm×2.1 mm, 2.7 μm, Phenomenex, Germany) using a gradient of Solvent A (methanol, water, formic acid, 400:400:1, w/w/w) against Solvent B (tert-butyl methyl ether, 2-propanol, methanol, formic acid, 400:200:100:1, w/w/w). Solvent C (methanol, 0.1M ammonium formate solution in water, 20:3, w/w) was added post-column during the elution time of triglycerides. Details of the gradient are given in Table 3.

TABLE 3
Liquid chromatography gradient used in the analytical protocol

| Analytical Gradient | | | | Post-Column Addition | | |
|---|---|---|---|---|---|---|
| Time | Flow Rate | Solvent A | Solvent B | Time | Flow | Solvent C |
| [min] | [μl/min] | [%] | [%] | [min] | [μl/min] | [%] |
| 0.00 | 400 | 100 | 0 | 0.00 | 0 | 100 |
| 0.10 | 400 | 100 | 0 | . . . gradient continued . . . | | |
| 0.20 | 400 | 100 | 0 | | | |
| 0.40 | 500 | 58 | 42 | | | |
| . . . gradient continued . . . | | | | 3.20 | 0 | 100 |
| 3.30 | 500 | 32 | 68 | 3.30 | 200 | 100 |
| 5.00 | 600 | 15 | 85 | . . . gradient continued . . . | | |
| . . . gradient continued . . . | | | | 5.30 | 200 | 100 |
| | | | | 5.40 | 0 | 100 |
| 5.50 | 600 | 15 | 85 | . . . gradient continued . . . | | |
| 5.70 | 400 | 100 | 0 | | | |
| 7.50 | 400 | 100 | 0 | 7.50 | 0 | 100 |

Mass spectrometry using multiple-reaction-monitoring (MRM):
The source parameters were: nebulizer gas, 50; heater gas, 60; curtain gas, 25; CAD gas, 4; ion spray voltage, 5500 V; temperature, 400° C.; pause between mass ranges, 5 ms; resolution Q1 and Q3, unit. MRM parameters, retention times, and molecular species contained in each metabolomic biomarker are listed in Table 4. Different MRM settings were used for the samples from heart failure patients and for the samples from patients with pulmonary disease (indicated by asterisks in Table 4).

TABLE 4
Retention times and MRM settings of the LC-MS/MS analysis used in the analytical protocol

| | Dwell | | | | | | | |
|---|---|---|---|---|---|---|---|---|
| Biomarker/Standard | Time | | | | | | | RT |
| Compound | [msec] | Q1 [Da] | Q3 [Da] | DP [V] | EP [V] | CE [V] | CXP [V] | [min] |
| PC4 | 10 | 758.6 | 184.1 | 85/60* | 10 | 70/85* | 15 | 1.72 |
| SM23 | 10 | 799.5 | 184.1 | 85/60* | 10 | 40 | 15 | 2.02 |
| SM27 | 10 | 813.5 | 184.1 | 60 | 10 | 40 | 15 | 2.06 |
| PC5-40 (internal standard | 10 | 818.6 | 184.1 | 85/60* | 10 | 40 | 15 | 2.38 |
| for SM, TAG, and CE, | | | | | | | | |
| collision energy 40 V) | | | | | | | | |
| PC5-70 (internal standard | 10 | 818.6 | 184.1 | 85/60* | 10 | 70/85* | 15 | 2.38 |
| for PC, collision energy | | | | | | | | |
| 70 V/85 V*) | | | | | | | | |
| CE C18:2 | 10 | 666.7 | 369.3 | 56/30* | 10 | 40/45* | 20 | 4.47 |
| OSS2 | 10 | 906.9 | 605.4 | 75/30* | 10 | 35 | 10 | 4.87 |

MRM settings (Q1, first mass transition; Q3, second mass transition; DP, declustering potential; EP, entrance potential; CE, collision energy; CXP, collision cell exit potential) and retention time (RT) for each metabolite biomarker.
*<setting used for heart failure samples>/<setting used pulmonary disease samples>

TABLE 5
Calibration solutions in MeOH/CH₂Cl₂ (2/1) for calibration samples preparation

| Concen- | | | | | | | | |
|---|---|---|---|---|---|---|---|---|
| tration | | STD1 | STD2 | STD3 | STD4 | STD5 | STD6 | STD7 |
| μg/ml | PC4 | 146.423 | 117.138 | 87.854 | 58.569 | 29.285 | 14.642 | 4.881 |
| | CE 18:2 | 365.760 | 292.608 | 219.456 | 146.304 | 73.152 | 36.576 | 12.192 |
| | SM27 | 12.358 | 9.886 | 7.415 | 4.943 | 2.472 | 1.236 | 0.4119 |
| | OSS2 | 1.974 | 1.5792 | 1.1844 | 0.7896 | 0.3948 | 0.1974 | 0.0658 |
| nmol/ml | PC4 | 193.1542 | 154.5234 | 115.8925 | 77.26169 | 38.63085 | 19.31542 | 6.438475 |
| | CE 18:2 | 563.5053 | 450.8042 | 338.1032 | 225.4021 | 112.7011 | 56.35053 | 18.78351 |
| | SM27 | 15.18735 | 12.14988 | 9.112412 | 6.074941 | 3.037471 | 1.518735 | 0.506245 |
| | OSS2 | 2.219314 | 1.775451 | 1.331588 | 0.887726 | 0.443863 | 0.221931 | 0.073977 |

TABLE 5a
Concentration ratios of the compounds within the calibration samples/calibration solutions for
calibration sample preparation, e.g. for the determination of PC4, CE18:2, SM23, and OSS2

| Ratio (from | | | | | | | |
|---|---|---|---|---|---|---|---|
| molar concen- | | | | | | | |
| trations; CE | | | | | | | |
| C18:2 set to | | | | | | | |
| “1”) | STD1 | STD2 | STD3 | STD4 | STD5 | STD6 | STD7 |
| PC 4 | 0.342773 | 0.274218 | 0.205664 | 0.137109 | 0.068555 | 0.034277 | 0.011426 |
| CE C18:2 | 1 | 0.8 | 0.6 | 0.4 | 0.2 | 0.1 | 0.033333 |
| SM27 | 0.026952 | 0.021561 | 0.016171 | 0.010781 | 0.00539 | 0.002695 | 0.000898 |
| OSS2 | 0.003938 | 0.003151 | 0.002363 | 0.001575 | 0.000788 | 0.000394 | 0.000131 |

### EXAMPLE 4

### Statistics

Prediction probabilities were calculated using Panel 1+NT-proBNP for the 67 DCMP patients and the 64 ICMP patients from the testing set (together referred to as HFrEF patients from the testing set) as well as for the 34 patients with pulmonary disease using the steps described below. Equivalent calculations can be performed using any of the panels listed in Tables 2 and 2A, with or without the additional inclusion of NT-proBNP.

Calculation of prediction probabilities:

The prediction probability for each sample was calculated as

$$p = \frac{1}{1 + {e}^{- ( {w}_{0} + {\sum }_{i = 1}^{n} {w}_{i} {\hat{x}}_{i} )}} ,$$

with the features \hat{x}ᵢ being

$${\hat{x}}_{i} = \frac{{x}_{i} - {m}_{i}}{{s}_{i}} \cdot {x}_{i}$$

are the log-transformed analytical results taking into account the units listed in Tables 6 and 6a, mᵢ, and sᵢ are feature-specific scaling factors, and wᵢ are the coefficients of the model (Tables 6 and 6a; w₀, intercept; w₁, coefficient for NT-proBNP; w₂ . . . wₙ, coefficients for the metabolomic features; n, total number of features in the panel plus NT-proBNP).

Feature-specific scaling factors mᵢ and sᵢ:

The feature-specific scaling factors mᵢ and sᵢ used for the samples from heart failure patients and healthy controls (Table 6) were chosen so that the log-transformed and scaled peak area ratios of all 823 subjects had a mean value of zero and a standard deviation of one. The feature-specific scaling factors mᵢ and sᵢ used for the samples from patients with pulmonary disease (Table 6A) had been determined from the samples from the heart failure patients and healthy controls employing an additional step involving an ultrapool sample consisting of commercially available human plasma, which was mixed well and split into a sufficient number of aliquots. Aliquots of the ultrapool sample were included with the samples from heart failure patients and healthy controls on each day of the analysis. In this way, peak area ratios for each biomarker in the aliquots of the ultrapool sample were obtained. In addition, absolute concentrations for each biomarker in further aliquots of the ultrapool sample were obtained using calibration samples containing known amounts of SM27 (for the quantification of SM23), PC4, and OSS2 in a similar way as described for the samples from patients with pulmonary disease. Absolute concentrations of SM23, PC4, and OSS2 in the samples from heart failure patients and healthy controls were calculated by dividing the peak area ratios of SM23, PC4, and OSS2 in each sample from a heart failure patient or a healthy control by the median of the respective peak area ratios in the aliquots of the ultrapool sample measured on the same day of the analysis as said patient sample, and then multiplying by the known concentrations of SM23, PC4, and OSS2, respectively, in the ultrapool sample. The feature-specific scaling factors mᵢ and sᵢ used for the samples from patients with pulmonary disease were then chosen so that the log-transformed and scaled absolute concentrations of all 823 heart failure patients and healthy controls had a mean value of zero and a standard deviation of one.

Coefficients wᵢ:

As described above, 534 randomly selected heart failure patients and healthy controls (stratified by subgroup) were taken as training set for the statistical analysis, and the remaining 289 heart failure patients and healthy controls were taken as testing set. The coefficients wᵢ were fitted on the 534 heart failure patients and healthy controls from the training set using the elastic net algorithm as implemented in the R package glmnet (Zou, H. and Hastie, T., 2003: Regression shrinkage and selection via the elastic net, with applications to microarrays. Journal of the Royal Statistical Society: Series B, 67, 301-320; Friedman, J., Hastie, T., and Tibshirani, R, 2010: Regularization Paths for Generalized Linear Models via Coordinate Descent. J. Stat. Softw. 33). The L1 and the L2 penalties were given equal weight.

Cuttoff value:

Using Panel 1+NT-proBNP, a prediction probability larger than the cutoff value of 0.738 was taken as positive diagnosis, and a prediction probability smaller than or equal to this cutoff value was taken as negative diagnosis for the respective sample. The cutoff value of 0.738 was chosen to obtain the same sensitivity for the detection of heart failure in the 534 heart failure patients and healthy controls in the training set as when using NT-proBNP alone. A cutoff value of 125 μg/ml was used for NT-proBNP alone.

Area under the curve (AUC):

The area under the curve (AUC) was calculated as a measure of diagnostic performance for Panel 1+NT-proBNP as well as for NT-proBNP alone. The AUC was determined from receiver-operating-characteristic (ROC) curves comparing the 131 HFrEF patients from the testing set to the 34 patients with pulmonary disease. Approximated ROC curves were calculated on the basis of a binormal model to compare the area-under-the-curve (Zhou X-H, McClish D K, Obuchowski N A. Statistical methods in diagnostic medicine: John Wiley & Sons; 2009). In order to achieve binormality, the prediction probabilities were transformed using the logit function and NT-proBNP values were log-transformed.

TABLE 6
Scaling factors mᵢ and sᵢ and coefficients wᵢ used for
the calculation of prediction probabilities with Panel
1 + NT-proBNP for the samples from heart failure
patients and healthy controls (semiquantitative input data).

| | | | Scaling Factors | | Coefficients |
|---|---|---|---|---|---|
| Feature | Index i | Units | mᵢ | sᵢ | wᵢ |
| Intercept | 0 | n.a. | n.a. | n.a. | 0.7602899 |
| NT- | 1 | pg/ml | 2.20238 | 0.6364517 | 1.5745605 |
| proBNP | | | | | |
| SM23 | 2 | Peak area | −0.5379916 | 0.115942 | −0.7158906 |
| | | ratio | | | |
| OSS2 | 3 | Peak area | −2.1316954 | 0.3863649 | 0.6762354 |
| | | ratio | | | |
| PC4 | 4 | Peak area | 0.8736775 | 0.0834807 | −0.4594183 |
| | | ratio | | | |

n.a., not applicable

TABLE 6a
Scaling factors mᵢ and sᵢ and coefficients wᵢ used for
the calculation of prediction probabilities with Panel
1 + NT-proBNP for the samples from patients suffering
from pulmonary disease (quantitative input data).

| | | | Scaling Factors | | Coefficients |
|---|---|---|---|---|---|
| Feature | Index i | Units | mᵢ | sᵢ | wᵢ |
| Intercept | 0 | n.a. | n.a. | n.a. | 0.7602899 |
| NT- | 1 | pg/ml | 2.20238 | 0.6364517 | 1.5745605 |
| proBNP | | | | | |
| SM23 | 2 | μg/dl | 3.106761 | 0.123062 | −0.7158906 |
| OSS2 | 3 | μg/dl | 2.12519 | 0.3926955 | 0.6762354 |
| PC4 | 4 | μg/dl | 4.583751 | 0.0892658 | −0.4594183 |

n.a., not applicable

### EXAMPLE 5

### Differentiation of HFrEF Patients from Patients with Pulmonary Diseases using Panel 1+NT-pro-BNP

In order to assess the performance of Panel 1+NT-proBNP for the differentiation of HFrEF patients from patients with pulmonary diseases, the 131 samples of patients with HFrEF from the testing set and 34 samples of patients with pulmonary diseases (pulmonary origin of dyspnea and absence of systolic and diastolic LV dysfunction according to the criteria described in Example 2) were selected for comparison.

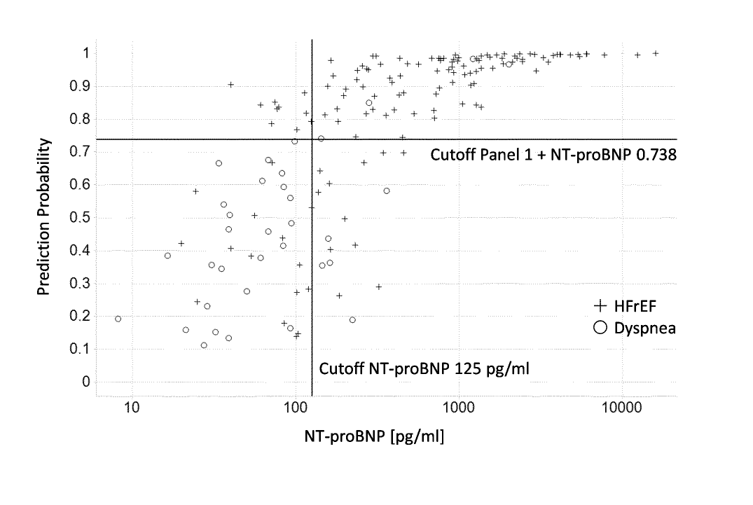

Using Panel 1+NT-proBNP with a cutoff value of 0.738, the specificity for correctly diagnosing the absence of heart failure in patients with pulmonary disease increased from 73.5% to 88.2% as compared to NT-proBNP alone due to an improved classification of 5 of the 34 patients with pulmonary disease (as HF negative). The respective patient classifications are shown in Table 7 and FIG. 1.

The AUC of a ROC curve was 0.887 for Panel 1+NT-proBNP and 0.873 for NT-proBNP alone.

TABLE 7
Heart failure classification using NT-proBNP or Panel
1 with NT-proBNP for the differentiation of heart failure
versus pulmonary disease (the terms “positive”
and “negative” refer to HF-diagnosis in all cases)

| | | NT-proBNP | | |
|---|---|---|---|---|
| HFrEF patients from the testing set | | Negative | Positive | Sensitivity |
| Panel 1 + NT-proBNP | Negative | 14 | 12 | 81.7% |
| | Positive | 10 | 95 | |
| | Sensitivity | 80.2% | | |
| Patients with pulmonary disease | | NT-proBNP | | |
| (considered HF negative) | | Negative | Positive | Specificity |
| Panel 1 + NT-proBNP | Negative | 25 | 5 | 73.5% |
| | Positive | 0 | 4 | |

| | |
|---|---|
| Specificity | 88.2% |

### EXAMPLE 6

### Differentiation of HFrEF Patients from Patients with Pulmonary Diseases using Panels 1-206+NT-pro-BNP

In order to assess the performance of Panels 1-206+NT-proBNP for the differentiation of HFrEF patients from patients with pulmonary diseases, 367 samples of patients with HFrEF and 239 samples from healthy controls (excluding dyspnea) were used as training set.

Panels 1-206 with resulting associated weights and scaling factors were then applied to the classification of the 34 samples of patients with pulmonary diseases (pulmonary origin of dyspnea and absence of systolic and diastolic LV dysfunction according to the criteria described in Example 2). The results are shown in Table 8.

Six different cut-offs for the separation of HFrEF and healthy controls panels were calculated for each panel based on the following considerations (see Table 8):
Youden: cut-off based on Youden index
optim.group sep.: cut-off for optimal group separation
Sensitivity fix 0.85: cut-off was selected such that the panel reaches a sensitivity of 85%
Sensitivity NT 0.8: cut-off was selected such that the panel reaches the same sensitivity as NT-proBNP at an NT-proBNP cut-off of 125 μg/mL, i.e. 80%
Specificity fix 0.9: cut-off was selected such that the panel reaches a specificity of 90%
Specificity NT 0.85: cut-off was selected such that the panel reaches the same specificity as NT-proBNP at an NT-proBNP cut-off of 125 μg/mL, i.e. 90%

All panels reached a specificity higher than 67% when classifying dyspnea patients with at least one of the cut-offs tested. Top-performing panels reached specificities above 90%.

TABLE 8
Diagnostic performance of panels 1-206 in heart failure
and dyspnea patients. AUC is the area under the receiver
operating characteristic-curve when differentiating HFrEF
patients from healthy controls (excluding dyspnea). Specificity
is the rate of dyspnea patients correctly classified as
not suffering from heart failure.

| Panel | Applied.Cut.off | Cut.off | AUC | Specificity |
|---|---|---|---|---|
| 1 | optim. group sep. | 0.540 | 0.968 | 0.529 |
| 1 | Sensitivity fix 0.85 | 0.671 | 0.968 | 0.794 |
| 1 | Sensitivity NT 0.8 | 0.757 | 0.968 | 0.882 |
| 1 | Specificity fix 0.9 | 0.469 | 0.968 | 0.441 |
| 1 | Specificity NT 0.85 | 0.408 | 0.968 | 0.353 |
| 1 | Youden | 0.591 | 0.968 | 0.647 |
| 2 | optim. group sep. | 0.541 | 0.966 | 0.500 |
| 2 | Sensitivity fix 0.85 | 0.641 | 0.966 | 0.735 |
| 2 | Sensitivity NT 0.8 | 0.739 | 0.966 | 0.794 |
| 2 | Specificity fix 0.9 | 0.482 | 0.966 | 0.441 |
| 2 | Specificity NT 0.85 | 0.413 | 0.966 | 0.324 |
| 2 | Youden | 0.630 | 0.966 | 0.735 |
| 3 | optim. group sep. | 0.539 | 0.967 | 0.471 |
| 3 | Sensitivity fix 0.85 | 0.668 | 0.967 | 0.676 |
| 3 | Sensitivity NT 0.8 | 0.759 | 0.967 | 0.853 |
| 3 | Specificity fix 0.9 | 0.480 | 0.967 | 0.412 |
| 3 | Specificity NT 0.85 | 0.426 | 0.967 | 0.324 |
| 3 | Youden | 0.595 | 0.967 | 0.559 |
| 4 | optim. group sep. | 0.541 | 0.966 | 0.500 |
| 4 | Sensitivity fix 0.85 | 0.641 | 0.966 | 0.735 |
| 4 | Sensitivity NT 0.8 | 0.739 | 0.966 | 0.794 |
| 4 | Specificity fix 0.9 | 0.482 | 0.966 | 0.441 |
| 4 | Specificity NT 0.85 | 0.413 | 0.966 | 0.324 |
| 4 | Youden | 0.630 | 0.966 | 0.735 |
| 5 | optim. group sep. | 0.539 | 0.967 | 0.500 |
| 5 | Sensitivity fix 0.85 | 0.668 | 0.967 | 0.706 |
| 5 | Sensitivity NT 0.8 | 0.757 | 0.967 | 0.824 |
| 5 | Specificity fix 0.9 | 0.479 | 0.967 | 0.382 |
| 5 | Specificity NT 0.85 | 0.430 | 0.967 | 0.324 |
| 5 | Youden | 0.530 | 0.967 | 0.471 |
| 6 | optim. group sep. | 0.541 | 0.966 | 0.559 |
| 6 | Sensitivity fix 0.85 | 0.641 | 0.966 | 0.676 |
| 6 | Sensitivity NT 0.8 | 0.733 | 0.966 | 0.765 |
| 6 | Specificity fix 0.9 | 0.510 | 0.966 | 0.529 |
| 6 | Specificity NT 0.85 | 0.425 | 0.966 | 0.324 |
| 6 | Youden | 0.539 | 0.966 | 0.559 |
| 7 | optim. group sep. | 0.540 | 0.966 | 0.559 |
| 7 | Sensitivity fix 0.85 | 0.653 | 0.966 | 0.706 |
| 7 | Sensitivity NT 0.8 | 0.751 | 0.966 | 0.794 |
| 7 | Specificity fix 0.9 | 0.502 | 0.966 | 0.500 |
| 7 | Specificity NT 0.85 | 0.423 | 0.966 | 0.382 |
| 7 | Youden | 0.652 | 0.966 | 0.676 |
| 8 | optim. group sep. | 0.540 | 0.966 | 0.559 |
| 8 | Sensitivity fix 0.85 | 0.653 | 0.966 | 0.706 |
| 8 | Sensitivity NT 0.8 | 0.751 | 0.966 | 0.794 |
| 8 | Specificity fix 0.9 | 0.502 | 0.966 | 0.500 |
| 8 | Specificity NT 0.85 | 0.423 | 0.966 | 0.382 |
| 8 | Youden | 0.652 | 0.966 | 0.676 |
| 9 | optim. group sep. | 0.539 | 0.967 | 0.471 |
| 9 | Sensitivity fix 0.85 | 0.682 | 0.967 | 0.647 |
| 9 | Sensitivity NT 0.8 | 0.773 | 0.967 | 0.824 |
| 9 | Specificity fix 0.9 | 0.500 | 0.967 | 0.441 |
| 9 | Specificity NT 0.85 | 0.428 | 0.967 | 0.294 |
| 9 | Youden | 0.635 | 0.967 | 0.588 |
| 10 | optim. group sep. | 0.541 | 0.965 | 0.618 |
| 10 | Sensitivity fix 0.85 | 0.640 | 0.965 | 0.735 |
| 10 | Sensitivity NT 0.8 | 0.724 | 0.965 | 0.882 |
| 10 | Specificity fix 0.9 | 0.523 | 0.965 | 0.559 |
| 10 | Specificity NT 0.85 | 0.409 | 0.965 | 0.412 |
| 10 | Youden | 0.619 | 0.965 | 0.735 |
| 11 | optim. group sep. | 0.539 | 0.966 | 0.559 |
| 11 | Sensitivity fix 0.85 | 0.647 | 0.966 | 0.647 |
| 11 | Sensitivity NT 0.8 | 0.738 | 0.966 | 0.735 |
| 11 | Specificity fix 0.9 | 0.490 | 0.966 | 0.441 |
| 11 | Specificity NT 0.85 | 0.435 | 0.966 | 0.382 |
| 11 | Youden | 0.618 | 0.966 | 0.618 |
| 12 | optim. group sep. | 0.541 | 0.964 | 0.500 |
| 12 | Sensitivity fix 0.85 | 0.632 | 0.964 | 0.676 |
| 12 | Sensitivity NT 0.8 | 0.731 | 0.964 | 0.765 |
| 12 | Specificity fix 0.9 | 0.527 | 0.964 | 0.500 |
| 12 | Specificity NT 0.85 | 0.428 | 0.964 | 0.382 |
| 12 | Youden | 0.626 | 0.964 | 0.676 |
| 13 | optim. group sep. | 0.540 | 0.968 | 0.529 |
| 13 | Sensitivity fix 0.85 | 0.671 | 0.968 | 0.794 |
| 13 | Sensitivity NT 0.8 | 0.757 | 0.968 | 0.882 |
| 13 | Specificity fix 0.9 | 0.469 | 0.968 | 0.441 |
| 13 | Specificity NT 0.85 | 0.407 | 0.968 | 0.353 |
| 13 | Youden | 0.591 | 0.968 | 0.647 |
| 14 | optim. group sep. | 0.540 | 0.966 | 0.559 |
| 14 | Sensitivity fix 0.85 | 0.633 | 0.966 | 0.735 |
| 14 | Sensitivity NT 0.8 | 0.736 | 0.966 | 0.794 |
| 14 | Specificity fix 0.9 | 0.502 | 0.966 | 0.500 |
| 14 | Specificity NT 0.85 | 0.428 | 0.966 | 0.412 |
| 14 | Youden | 0.611 | 0.966 | 0.706 |
| 15 | optim. group sep. | 0.540 | 0.967 | 0.529 |
| 15 | Sensitivity fix 0.85 | 0.670 | 0.967 | 0.676 |
| 15 | Sensitivity NT 0.8 | 0.749 | 0.967 | 0.824 |
| 15 | Specificity fix 0.9 | 0.479 | 0.967 | 0.382 |
| 15 | Specificity NT 0.85 | 0.439 | 0.967 | 0.353 |
| 15 | Youden | 0.542 | 0.967 | 0.529 |
| 16 | optim. group sep. | 0.540 | 0.965 | 0.559 |
| 16 | Sensitivity fix 0.85 | 0.656 | 0.965 | 0.647 |
| 16 | Sensitivity NT 0.8 | 0.749 | 0.965 | 0.765 |
| 16 | Specificity fix 0.9 | 0.526 | 0.965 | 0.559 |
| 16 | Specificity NT 0.85 | 0.427 | 0.965 | 0.382 |
| 16 | Youden | 0.627 | 0.965 | 0.647 |
| 17 | optim. group sep. | 0.539 | 0.967 | 0.471 |
| 17 | Sensitivity fix 0.85 | 0.670 | 0.967 | 0.676 |
| 17 | Sensitivity NT 0.8 | 0.756 | 0.967 | 0.794 |
| 17 | Specificity fix 0.9 | 0.475 | 0.967 | 0.382 |
| 17 | Specificity NT 0.85 | 0.432 | 0.967 | 0.324 |
| 17 | Youden | 0.593 | 0.967 | 0.559 |
| 18 | optim. group sep. | 0.541 | 0.966 | 0.559 |
| 18 | Sensitivity fix 0.85 | 0.641 | 0.966 | 0.676 |
| 18 | Sensitivity NT 0.8 | 0.733 | 0.966 | 0.765 |
| 18 | Specificity fix 0.9 | 0.510 | 0.966 | 0.529 |
| 18 | Specificity NT 0.85 | 0.425 | 0.966 | 0.324 |
| 18 | Youden | 0.539 | 0.966 | 0.559 |
| 19 | optim. group sep. | 0.540 | 0.966 | 0.559 |
| 19 | Sensitivity fix 0.85 | 0.653 | 0.966 | 0.706 |
| 19 | Sensitivity NT 0.8 | 0.751 | 0.966 | 0.794 |
| 19 | Specificity fix 0.9 | 0.502 | 0.966 | 0.500 |
| 19 | Specificity NT 0.85 | 0.423 | 0.966 | 0.382 |
| 19 | Youden | 0.652 | 0.966 | 0.676 |
| 20 | optim. group sep. | 0.541 | 0.966 | 0.500 |
| 20 | Sensitivity fix 0.85 | 0.641 | 0.966 | 0.735 |
| 20 | Sensitivity NT 0.8 | 0.739 | 0.966 | 0.794 |
| 20 | Specificity fix 0.9 | 0.482 | 0.966 | 0.441 |
| 20 | Specificity NT 0.85 | 0.413 | 0.966 | 0.324 |
| 20 | Youden | 0.630 | 0.966 | 0.735 |
| 21 | optim. group sep. | 0.540 | 0.965 | 0.441 |
| 21 | Sensitivity fix 0.85 | 0.642 | 0.965 | 0.735 |
| 21 | Sensitivity NT 0.8 | 0.733 | 0.965 | 0.765 |
| 21 | Specificity fix 0.9 | 0.487 | 0.965 | 0.324 |
| 21 | Specificity NT 0.85 | 0.406 | 0.965 | 0.324 |
| 21 | Youden | 0.659 | 0.965 | 0.735 |
| 22 | optim. group sep. | 0.541 | 0.966 | 0.500 |
| 22 | Sensitivity fix 0.85 | 0.641 | 0.966 | 0.735 |
| 22 | Sensitivity NT 0.8 | 0.739 | 0.966 | 0.794 |
| 22 | Specificity fix 0.9 | 0.482 | 0.966 | 0.441 |
| 22 | Specificity NT 0.85 | 0.413 | 0.966 | 0.324 |
| 22 | Youden | 0.630 | 0.966 | 0.735 |
| 23 | optim. group sep. | 0.540 | 0.965 | 0.441 |
| 23 | Sensitivity fix 0.85 | 0.642 | 0.965 | 0.735 |
| 23 | Sensitivity NT 0.8 | 0.733 | 0.965 | 0.765 |
| 23 | Specificity fix 0.9 | 0.487 | 0.965 | 0.324 |
| 23 | Specificity NT 0.85 | 0.406 | 0.965 | 0.324 |
| 23 | Youden | 0.659 | 0.965 | 0.735 |
| 24 | optim. group sep. | 0.543 | 0.961 | 0.559 |
| 24 | Sensitivity fix 0.85 | 0.648 | 0.961 | 0.794 |
| 24 | Sensitivity NT 0.8 | 0.708 | 0.961 | 0.824 |
| 24 | Specificity fix 0.9 | 0.534 | 0.961 | 0.529 |
| 24 | Specificity NT 0.85 | 0.455 | 0.961 | 0.412 |
| 24 | Youden | 0.616 | 0.961 | 0.735 |
| 25 | optim. group sep. | 0.538 | 0.970 | 0.559 |
| 25 | Sensitivity fix 0.85 | 0.667 | 0.970 | 0.765 |
| 25 | Sensitivity NT 0.8 | 0.788 | 0.970 | 0.882 |
| 25 | Specificity fix 0.9 | 0.479 | 0.970 | 0.471 |
| 25 | Specificity NT 0.85 | 0.410 | 0.970 | 0.412 |
| 25 | Youden | 0.547 | 0.970 | 0.559 |
| 26 | optim. group sep. | 0.539 | 0.969 | 0.618 |
| 26 | Sensitivity fix 0.85 | 0.676 | 0.969 | 0.765 |
| 26 | Sensitivity NT 0.8 | 0.769 | 0.969 | 0.912 |
| 26 | Specificity fix 0.9 | 0.470 | 0.969 | 0.471 |
| 26 | Specificity NT 0.85 | 0.403 | 0.969 | 0.382 |
| 26 | Youden | 0.579 | 0.969 | 0.647 |
| 27 | optim. group sep. | 0.539 | 0.968 | 0.471 |
| 27 | Sensitivity fix 0.85 | 0.673 | 0.968 | 0.618 |
| 27 | Sensitivity NT 0.8 | 0.761 | 0.968 | 0.824 |
| 27 | Specificity fix 0.9 | 0.484 | 0.968 | 0.382 |
| 27 | Specificity NT 0.85 | 0.436 | 0.968 | 0.324 |
| 27 | Youden | 0.564 | 0.968 | 0.559 |
| 28 | optim. group sep. | 0.540 | 0.967 | 0.559 |
| 28 | Sensitivity fix 0.85 | 0.658 | 0.967 | 0.735 |
| 28 | Sensitivity NT 0.8 | 0.760 | 0.967 | 0.853 |
| 28 | Specificity fix 0.9 | 0.517 | 0.967 | 0.529 |
| 28 | Specificity NT 0.85 | 0.421 | 0.967 | 0.382 |
| 28 | Youden | 0.575 | 0.967 | 0.559 |
| 29 | optim. group sep. | 0.540 | 0.967 | 0.441 |
| 29 | Sensitivity fix 0.85 | 0.677 | 0.967 | 0.735 |
| 29 | Sensitivity NT 0.8 | 0.756 | 0.967 | 0.824 |
| 29 | Specificity fix 0.9 | 0.501 | 0.967 | 0.412 |
| 29 | Specificity NT 0.85 | 0.427 | 0.967 | 0.324 |
| 29 | Youden | 0.528 | 0.967 | 0.441 |
| 30 | optim. group sep. | 0.541 | 0.963 | 0.529 |
| 30 | Sensitivity fix 0.85 | 0.645 | 0.963 | 0.706 |
| 30 | Sensitivity NT 0.8 | 0.711 | 0.963 | 0.765 |
| 30 | Specificity fix 0.9 | 0.514 | 0.963 | 0.471 |
| 30 | Specificity NT 0.85 | 0.417 | 0.963 | 0.294 |
| 30 | Youden | 0.545 | 0.963 | 0.529 |
| 31 | optim. group sep. | 0.539 | 0.969 | 0.529 |
| 31 | Sensitivity fix 0.85 | 0.696 | 0.969 | 0.765 |
| 31 | Sensitivity NT 0.8 | 0.760 | 0.969 | 0.853 |
| 31 | Specificity fix 0.9 | 0.496 | 0.969 | 0.471 |
| 31 | Specificity NT 0.85 | 0.414 | 0.969 | 0.353 |
| 31 | Youden | 0.570 | 0.969 | 0.647 |
| 32 | optim. group sep. | 0.541 | 0.966 | 0.500 |
| 32 | Sensitivity fix 0.85 | 0.641 | 0.966 | 0.735 |
| 32 | Sensitivity NT 0.8 | 0.739 | 0.966 | 0.794 |
| 32 | Specificity fix 0.9 | 0.482 | 0.966 | 0.441 |
| 32 | Specificity NT 0.85 | 0.413 | 0.966 | 0.324 |
| 32 | Youden | 0.630 | 0.966 | 0.735 |
| 33 | optim. group sep. | 0.540 | 0.967 | 0.441 |
| 33 | Sensitivity fix 0.85 | 0.675 | 0.967 | 0.706 |
| 33 | Sensitivity NT 0.8 | 0.755 | 0.967 | 0.853 |
| 33 | Specificity fix 0.9 | 0.500 | 0.967 | 0.382 |
| 33 | Specificity NT 0.85 | 0.418 | 0.967 | 0.324 |
| 33 | Youden | 0.528 | 0.967 | 0.412 |
| 34 | optim. group sep. | 0.539 | 0.967 | 0.559 |
| 34 | Sensitivity fix 0.85 | 0.660 | 0.967 | 0.735 |
| 34 | Sensitivity NT 0.8 | 0.766 | 0.967 | 0.853 |
| 34 | Specificity fix 0.9 | 0.491 | 0.967 | 0.471 |
| 34 | Specificity NT 0.85 | 0.410 | 0.967 | 0.353 |
| 34 | Youden | 0.572 | 0.967 | 0.559 |
| 35 | optim. group sep. | 0.539 | 0.967 | 0.471 |
| 35 | Sensitivity fix 0.85 | 0.667 | 0.967 | 0.618 |
| 35 | Sensitivity NT 0.8 | 0.753 | 0.967 | 0.794 |
| 35 | Specificity fix 0.9 | 0.475 | 0.967 | 0.382 |
| 35 | Specificity NT 0.85 | 0.434 | 0.967 | 0.324 |
| 35 | Youden | 0.556 | 0.967 | 0.471 |
| 36 | optim. group sep. | 0.541 | 0.963 | 0.529 |
| 36 | Sensitivity fix 0.85 | 0.645 | 0.963 | 0.706 |
| 36 | Sensitivity NT 0.8 | 0.711 | 0.963 | 0.765 |
| 36 | Specificity fix 0.9 | 0.514 | 0.963 | 0.471 |
| 36 | Specificity NT 0.85 | 0.417 | 0.963 | 0.294 |
| 36 | Youden | 0.545 | 0.963 | 0.529 |
| 37 | optim. group sep. | 0.542 | 0.961 | 0.618 |
| 37 | Sensitivity fix 0.85 | 0.644 | 0.961 | 0.735 |
| 37 | Sensitivity NT 0.8 | 0.711 | 0.961 | 0.853 |
| 37 | Specificity fix 0.9 | 0.539 | 0.961 | 0.618 |
| 37 | Specificity NT 0.85 | 0.439 | 0.961 | 0.471 |
| 37 | Youden | 0.628 | 0.961 | 0.706 |
| 38 | optim. group sep. | 0.541 | 0.964 | 0.618 |
| 38 | Sensitivity fix 0.85 | 0.655 | 0.964 | 0.765 |
| 38 | Sensitivity NT 0.8 | 0.722 | 0.964 | 0.853 |
| 38 | Specificity fix 0.9 | 0.540 | 0.964 | 0.618 |
| 38 | Specificity NT 0.85 | 0.415 | 0.964 | 0.471 |
| 38 | Youden | 0.655 | 0.964 | 0.765 |
| 39 | optim. group sep. | 0.539 | 0.967 | 0.471 |
| 39 | Sensitivity fix 0.85 | 0.683 | 0.967 | 0.647 |
| 39 | Sensitivity NT 0.8 | 0.773 | 0.967 | 0.824 |
| 39 | Specificity fix 0.9 | 0.502 | 0.967 | 0.441 |
| 39 | Specificity NT 0.85 | 0.428 | 0.967 | 0.294 |
| 39 | Youden | 0.637 | 0.967 | 0.588 |
| 40 | optim. group sep. | 0.539 | 0.967 | 0.559 |
| 40 | Sensitivity fix 0.85 | 0.660 | 0.967 | 0.735 |
| 40 | Sensitivity NT 0.8 | 0.766 | 0.967 | 0.853 |
| 40 | Specificity fix 0.9 | 0.491 | 0.967 | 0.471 |
| 40 | Specificity NT 0.85 | 0.410 | 0.967 | 0.353 |
| 40 | Youden | 0.572 | 0.967 | 0.559 |
| 41 | optim. group sep. | 0.536 | 0.968 | 0.529 |
| 41 | Sensitivity fix 0.85 | 0.670 | 0.968 | 0.647 |
| 41 | Sensitivity NT 0.8 | 0.786 | 0.968 | 0.853 |
| 41 | Specificity fix 0.9 | 0.505 | 0.968 | 0.500 |
| 41 | Specificity NT 0.85 | 0.436 | 0.968 | 0.441 |
| 41 | Youden | 0.622 | 0.968 | 0.647 |
| 42 | optim. group sep. | 0.541 | 0.964 | 0.500 |
| 42 | Sensitivity fix 0.85 | 0.632 | 0.964 | 0.676 |
| 42 | Sensitivity NT 0.8 | 0.731 | 0.964 | 0.765 |
| 42 | Specificity fix 0.9 | 0.527 | 0.964 | 0.500 |
| 42 | Specificity NT 0.85 | 0.428 | 0.964 | 0.382 |
| 42 | Youden | 0.626 | 0.964 | 0.676 |
| 43 | optim. group sep. | 0.540 | 0.965 | 0.559 |
| 43 | Sensitivity fix 0.85 | 0.656 | 0.965 | 0.647 |
| 43 | Sensitivity NT 0.8 | 0.749 | 0.965 | 0.765 |
| 43 | Specificity fix 0.9 | 0.526 | 0.965 | 0.559 |
| 43 | Specificity NT 0.85 | 0.427 | 0.965 | 0.382 |
| 43 | Youden | 0.627 | 0.965 | 0.647 |
| 44 | optim. group sep. | 0.540 | 0.965 | 0.559 |
| 44 | Sensitivity fix 0.85 | 0.656 | 0.965 | 0.647 |
| 44 | Sensitivity NT 0.8 | 0.748 | 0.965 | 0.765 |
| 44 | Specificity fix 0.9 | 0.526 | 0.965 | 0.559 |
| 44 | Specificity NT 0.85 | 0.427 | 0.965 | 0.382 |
| 44 | Youden | 0.627 | 0.965 | 0.647 |
| 45 | optim. group sep. | 0.540 | 0.965 | 0.559 |
| 45 | Sensitivity fix 0.85 | 0.644 | 0.965 | 0.647 |
| 45 | Sensitivity NT 0.8 | 0.742 | 0.965 | 0.765 |
| 45 | Specificity fix 0.9 | 0.529 | 0.965 | 0.529 |
| 45 | Specificity NT 0.85 | 0.432 | 0.965 | 0.353 |
| 45 | Youden | 0.612 | 0.965 | 0.647 |
| 46 | optim. group sep. | 0.540 | 0.965 | 0.559 |
| 46 | Sensitivity fix 0.85 | 0.656 | 0.965 | 0.647 |
| 46 | Sensitivity NT 0.8 | 0.748 | 0.965 | 0.765 |
| 46 | Specificity fix 0.9 | 0.526 | 0.965 | 0.559 |
| 46 | Specificity NT 0.85 | 0.427 | 0.965 | 0.382 |
| 46 | Youden | 0.627 | 0.965 | 0.647 |
| 47 | optim. group sep. | 0.539 | 0.967 | 0.471 |
| 47 | Sensitivity fix 0.85 | 0.683 | 0.967 | 0.647 |
| 47 | Sensitivity NT 0.8 | 0.769 | 0.967 | 0.824 |
| 47 | Specificity fix 0.9 | 0.505 | 0.967 | 0.412 |
| 47 | Specificity NT 0.85 | 0.429 | 0.967 | 0.294 |
| 47 | Youden | 0.583 | 0.967 | 0.529 |
| 48 | optim. group sep. | 0.540 | 0.965 | 0.559 |
| 48 | Sensitivity fix 0.85 | 0.656 | 0.965 | 0.647 |
| 48 | Sensitivity NT 0.8 | 0.748 | 0.965 | 0.765 |
| 48 | Specificity fix 0.9 | 0.526 | 0.965 | 0.559 |
| 48 | Specificity NT 0.85 | 0.427 | 0.965 | 0.382 |
| 48 | Youden | 0.627 | 0.965 | 0.647 |
| 49 | optim. group sep. | 0.540 | 0.965 | 0.559 |
| 49 | Sensitivity fix 0.85 | 0.643 | 0.965 | 0.647 |
| 49 | Sensitivity NT 0.8 | 0.744 | 0.965 | 0.765 |
| 49 | Specificity fix 0.9 | 0.526 | 0.965 | 0.529 |
| 49 | Specificity NT 0.85 | 0.428 | 0.965 | 0.353 |
| 49 | Youden | 0.615 | 0.965 | 0.647 |
| 50 | optim. group sep. | 0.540 | 0.965 | 0.559 |
| 50 | Sensitivity fix 0.85 | 0.656 | 0.965 | 0.647 |
| 50 | Sensitivity NT 0.8 | 0.748 | 0.965 | 0.765 |
| 50 | Specificity fix 0.9 | 0.526 | 0.965 | 0.559 |
| 50 | Specificity NT 0.85 | 0.427 | 0.965 | 0.382 |
| 50 | Youden | 0.627 | 0.965 | 0.647 |
| 51 | optim. group sep. | 0.540 | 0.966 | 0.559 |
| 51 | Sensitivity fix 0.85 | 0.653 | 0.966 | 0.706 |
| 51 | Sensitivity NT 0.8 | 0.751 | 0.966 | 0.794 |
| 51 | Specificity fix 0.9 | 0.502 | 0.966 | 0.500 |
| 51 | Specificity NT 0.85 | 0.423 | 0.966 | 0.382 |
| 51 | Youden | 0.652 | 0.966 | 0.676 |
| 52 | optim. group sep. | 0.540 | 0.966 | 0.559 |
| 52 | Sensitivity fix 0.85 | 0.653 | 0.966 | 0.706 |
| 52 | Sensitivity NT 0.8 | 0.751 | 0.966 | 0.794 |
| 52 | Specificity fix 0.9 | 0.502 | 0.966 | 0.500 |
| 52 | Specificity NT 0.85 | 0.423 | 0.966 | 0.382 |
| 52 | Youden | 0.652 | 0.966 | 0.676 |
| 53 | optim. group sep. | 0.539 | 0.967 | 0.500 |
| 53 | Sensitivity fix 0.85 | 0.667 | 0.967 | 0.676 |
| 53 | Sensitivity NT 0.8 | 0.757 | 0.967 | 0.824 |
| 53 | Specificity fix 0.9 | 0.480 | 0.967 | 0.382 |
| 53 | Specificity NT 0.85 | 0.426 | 0.967 | 0.324 |
| 53 | Youden | 0.537 | 0.967 | 0.500 |
| 54 | optim. group sep. | 0.540 | 0.965 | 0.559 |
| 54 | Sensitivity fix 0.85 | 0.656 | 0.965 | 0.647 |
| 54 | Sensitivity NT 0.8 | 0.748 | 0.965 | 0.765 |
| 54 | Specificity fix 0.9 | 0.526 | 0.965 | 0.559 |
| 54 | Specificity NT 0.85 | 0.427 | 0.965 | 0.382 |
| 54 | Youden | 0.627 | 0.965 | 0.647 |
| 55 | optim. group sep. | 0.539 | 0.967 | 0.500 |
| 55 | Sensitivity fix 0.85 | 0.668 | 0.967 | 0.706 |
| 55 | Sensitivity NT 0.8 | 0.757 | 0.967 | 0.824 |
| 55 | Specificity fix 0.9 | 0.479 | 0.967 | 0.382 |
| 55 | Specificity NT 0.85 | 0.430 | 0.967 | 0.324 |
| 55 | Youden | 0.530 | 0.967 | 0.471 |
| 56 | optim. group sep. | 0.540 | 0.965 | 0.559 |
| 56 | Sensitivity fix 0.85 | 0.656 | 0.965 | 0.647 |
| 56 | Sensitivity NT 0.8 | 0.748 | 0.965 | 0.765 |
| 56 | Specificity fix 0.9 | 0.526 | 0.965 | 0.559 |
| 56 | Specificity NT 0.85 | 0.427 | 0.965 | 0.382 |
| 56 | Youden | 0.627 | 0.965 | 0.647 |
| 57 | optim. group sep. | 0.539 | 0.969 | 0.529 |
| 57 | Sensitivity fix 0.85 | 0.696 | 0.969 | 0.765 |
| 57 | Sensitivity NT 0.8 | 0.760 | 0.969 | 0.853 |
| 57 | Specificity fix 0.9 | 0.496 | 0.969 | 0.471 |
| 57 | Specificity NT 0.85 | 0.414 | 0.969 | 0.353 |
| 57 | Youden | 0.570 | 0.969 | 0.647 |
| 58 | optim. group sep. | 0.537 | 0.969 | 0.529 |
| 58 | Sensitivity fix 0.85 | 0.686 | 0.969 | 0.706 |
| 58 | Sensitivity NT 0.8 | 0.787 | 0.969 | 0.882 |
| 58 | Specificity fix 0.9 | 0.487 | 0.969 | 0.471 |
| 58 | Specificity NT 0.85 | 0.421 | 0.969 | 0.382 |
| 58 | Youden | 0.600 | 0.969 | 0.559 |
| 59 | optim. group sep. | 0.539 | 0.968 | 0.647 |
| 59 | Sensitivity fix 0.85 | 0.659 | 0.968 | 0.765 |
| 59 | Sensitivity NT 0.8 | 0.774 | 0.968 | 0.882 |
| 59 | Specificity fix 0.9 | 0.508 | 0.968 | 0.647 |
| 59 | Specificity NT 0.85 | 0.432 | 0.968 | 0.471 |
| 59 | Youden | 0.584 | 0.968 | 0.676 |
| 60 | optim. group sep. | 0.539 | 0.968 | 0.500 |
| 60 | Sensitivity fix 0.85 | 0.677 | 0.968 | 0.706 |
| 60 | Sensitivity NT 0.8 | 0.766 | 0.968 | 0.882 |
| 60 | Specificity fix 0.9 | 0.486 | 0.968 | 0.353 |
| 60 | Specificity NT 0.85 | 0.397 | 0.968 | 0.324 |
| 60 | Youden | 0.509 | 0.968 | 0.441 |
| 61 | optim. group sep. | 0.540 | 0.966 | 0.559 |
| 61 | Sensitivity fix 0.85 | 0.653 | 0.966 | 0.706 |
| 61 | Sensitivity NT 0.8 | 0.751 | 0.966 | 0.794 |
| 61 | Specificity fix 0.9 | 0.502 | 0.966 | 0.500 |
| 61 | Specificity NT 0.85 | 0.423 | 0.966 | 0.382 |
| 61 | Youden | 0.652 | 0.966 | 0.676 |
| 62 | optim. group sep. | 0.537 | 0.968 | 0.471 |
| 62 | Sensitivity fix 0.85 | 0.683 | 0.968 | 0.676 |
| 62 | Sensitivity NT 0.8 | 0.773 | 0.968 | 0.882 |
| 62 | Specificity fix 0.9 | 0.480 | 0.968 | 0.412 |
| 62 | Specificity NT 0.85 | 0.441 | 0.968 | 0.412 |
| 62 | Youden | 0.613 | 0.968 | 0.559 |
| 63 | optim. group sep. | 0.540 | 0.965 | 0.559 |
| 63 | Sensitivity fix 0.85 | 0.656 | 0.965 | 0.647 |
| 63 | Sensitivity NT 0.8 | 0.748 | 0.965 | 0.765 |
| 63 | Specificity fix 0.9 | 0.526 | 0.965 | 0.559 |
| 63 | Specificity NT 0.85 | 0.427 | 0.965 | 0.382 |
| 63 | Youden | 0.627 | 0.965 | 0.647 |
| 64 | optim. group sep. | 0.540 | 0.967 | 0.559 |
| 64 | Sensitivity fix 0.85 | 0.680 | 0.967 | 0.765 |
| 64 | Sensitivity NT 0.8 | 0.756 | 0.967 | 0.824 |
| 64 | Specificity fix 0.9 | 0.497 | 0.967 | 0.441 |
| 64 | Specificity NT 0.85 | 0.420 | 0.967 | 0.353 |
| 64 | Youden | 0.535 | 0.967 | 0.559 |
| 65 | optim. group sep. | 0.539 | 0.967 | 0.559 |
| 65 | Sensitivity fix 0.85 | 0.660 | 0.967 | 0.735 |
| 65 | Sensitivity NT 0.8 | 0.766 | 0.967 | 0.853 |
| 65 | Specificity fix 0.9 | 0.491 | 0.967 | 0.471 |
| 65 | Specificity NT 0.85 | 0.410 | 0.967 | 0.353 |
| 65 | Youden | 0.572 | 0.967 | 0.559 |
| 66 | optim. group sep. | 0.539 | 0.968 | 0.529 |
| 66 | Sensitivity fix 0.85 | 0.677 | 0.968 | 0.735 |
| 66 | Sensitivity NT 0.8 | 0.775 | 0.968 | 0.794 |
| 66 | Specificity fix 0.9 | 0.503 | 0.968 | 0.500 |
| 66 | Specificity NT 0.85 | 0.418 | 0.968 | 0.353 |
| 66 | Youden | 0.617 | 0.968 | 0.618 |
| 67 | optim. group sep. | 0.539 | 0.968 | 0.559 |
| 67 | Sensitivity fix 0.85 | 0.678 | 0.968 | 0.735 |
| 67 | Sensitivity NT 0.8 | 0.779 | 0.968 | 0.794 |
| 67 | Specificity fix 0.9 | 0.500 | 0.968 | 0.500 |
| 67 | Specificity NT 0.85 | 0.425 | 0.968 | 0.382 |
| 67 | Youden | 0.583 | 0.968 | 0.588 |
| 68 | optim. group sep. | 0.539 | 0.968 | 0.559 |
| 68 | Sensitivity fix 0.85 | 0.672 | 0.968 | 0.735 |
| 68 | Sensitivity NT 0.8 | 0.771 | 0.968 | 0.794 |
| 68 | Specificity fix 0.9 | 0.495 | 0.968 | 0.500 |
| 68 | Specificity NT 0.85 | 0.426 | 0.968 | 0.382 |
| 68 | Youden | 0.582 | 0.968 | 0.588 |
| 69 | optim. group sep. | 0.539 | 0.968 | 0.559 |
| 69 | Sensitivity fix 0.85 | 0.678 | 0.968 | 0.735 |
| 69 | Sensitivity NT 0.8 | 0.779 | 0.968 | 0.794 |
| 69 | Specificity fix 0.9 | 0.500 | 0.968 | 0.500 |
| 69 | Specificity NT 0.85 | 0.425 | 0.968 | 0.382 |
| 69 | Youden | 0.583 | 0.968 | 0.588 |
| 70 | optim. group sep. | 0.539 | 0.967 | 0.471 |
| 70 | Sensitivity fix 0.85 | 0.670 | 0.967 | 0.676 |
| 70 | Sensitivity NT 0.8 | 0.756 | 0.967 | 0.794 |
| 70 | Specificity fix 0.9 | 0.475 | 0.967 | 0.382 |
| 70 | Specificity NT 0.85 | 0.432 | 0.967 | 0.324 |
| 70 | Youden | 0.594 | 0.967 | 0.559 |
| 71 | optim. group sep. | 0.539 | 0.968 | 0.559 |
| 71 | Sensitivity fix 0.85 | 0.678 | 0.968 | 0.735 |
| 71 | Sensitivity NT 0.8 | 0.779 | 0.968 | 0.794 |
| 71 | Specificity fix 0.9 | 0.500 | 0.968 | 0.500 |
| 71 | Specificity NT 0.85 | 0.425 | 0.968 | 0.382 |
| 71 | Youden | 0.583 | 0.968 | 0.588 |
| 72 | optim. group sep. | 0.539 | 0.968 | 0.559 |
| 72 | Sensitivity fix 0.85 | 0.666 | 0.968 | 0.794 |
| 72 | Sensitivity NT 0.8 | 0.767 | 0.968 | 0.882 |
| 72 | Specificity fix 0.9 | 0.474 | 0.968 | 0.441 |
| 72 | Specificity NT 0.85 | 0.423 | 0.968 | 0.412 |
| 72 | Youden | 0.606 | 0.968 | 0.676 |
| 73 | optim. group sep. | 0.538 | 0.969 | 0.559 |
| 73 | Sensitivity fix 0.85 | 0.665 | 0.969 | 0.765 |
| 73 | Sensitivity NT 0.8 | 0.787 | 0.969 | 0.853 |
| 73 | Specificity fix 0.9 | 0.484 | 0.969 | 0.471 |
| 73 | Specificity NT 0.85 | 0.414 | 0.969 | 0.412 |
| 73 | Youden | 0.565 | 0.969 | 0.588 |
| 74 | optim. group sep. | 0.538 | 0.970 | 0.559 |
| 74 | Sensitivity fix 0.85 | 0.667 | 0.970 | 0.765 |
| 74 | Sensitivity NT 0.8 | 0.788 | 0.970 | 0.882 |
| 74 | Specificity fix 0.9 | 0.479 | 0.970 | 0.471 |
| 74 | Specificity NT 0.85 | 0.410 | 0.970 | 0.412 |
| 74 | Youden | 0.547 | 0.970 | 0.559 |
| 75 | optim. group sep. | 0.539 | 0.968 | 0.471 |
| 75 | Sensitivity fix 0.85 | 0.673 | 0.968 | 0.618 |
| 75 | Sensitivity NT 0.8 | 0.761 | 0.968 | 0.824 |
| 75 | Specificity fix 0.9 | 0.488 | 0.968 | 0.382 |
| 75 | Specificity NT 0.85 | 0.436 | 0.968 | 0.324 |
| 75 | Youden | 0.565 | 0.968 | 0.559 |
| 76 | optim. group sep. | 0.539 | 0.968 | 0.559 |
| 76 | Sensitivity fix 0.85 | 0.678 | 0.968 | 0.735 |
| 76 | Sensitivity NT 0.8 | 0.779 | 0.968 | 0.794 |
| 76 | Specificity fix 0.9 | 0.500 | 0.968 | 0.500 |
| 76 | Specificity NT 0.85 | 0.425 | 0.968 | 0.382 |
| 76 | Youden | 0.583 | 0.968 | 0.588 |
| 77 | optim. group sep. | 0.539 | 0.967 | 0.500 |
| 77 | Sensitivity fix 0.85 | 0.668 | 0.967 | 0.706 |
| 77 | Sensitivity NT 0.8 | 0.757 | 0.967 | 0.824 |
| 77 | Specificity fix 0.9 | 0.479 | 0.967 | 0.382 |
| 77 | Specificity NT 0.85 | 0.430 | 0.967 | 0.324 |
| 77 | Youden | 0.530 | 0.967 | 0.471 |
| 78 | optim. group sep. | 0.541 | 0.966 | 0.559 |
| 78 | Sensitivity fix 0.85 | 0.641 | 0.966 | 0.676 |
| 78 | Sensitivity NT 0.8 | 0.733 | 0.966 | 0.765 |
| 78 | Specificity fix 0.9 | 0.510 | 0.966 | 0.529 |
| 78 | Specificity NT 0.85 | 0.425 | 0.966 | 0.324 |
| 78 | Youden | 0.539 | 0.966 | 0.559 |
| 79 | optim. group sep. | 0.538 | 0.969 | 0.529 |
| 79 | Sensitivity fix 0.85 | 0.690 | 0.969 | 0.765 |
| 79 | Sensitivity NT 0.8 | 0.780 | 0.969 | 0.853 |
| 79 | Specificity fix 0.9 | 0.485 | 0.969 | 0.441 |
| 79 | Specificity NT 0.85 | 0.412 | 0.969 | 0.382 |
| 79 | Youden | 0.587 | 0.969 | 0.559 |
| 80 | optim. group sep. | 0.540 | 0.965 | 0.471 |
| 80 | Sensitivity fix 0.85 | 0.652 | 0.965 | 0.618 |
| 80 | Sensitivity NT 0.8 | 0.743 | 0.965 | 0.765 |
| 80 | Specificity fix 0.9 | 0.514 | 0.965 | 0.412 |
| 80 | Specificity NT 0.85 | 0.440 | 0.965 | 0.265 |
| 80 | Youden | 0.586 | 0.965 | 0.500 |
| 81 | optim. group sep. | 0.539 | 0.969 | 0.529 |
| 81 | Sensitivity fix 0.85 | 0.675 | 0.969 | 0.735 |
| 81 | Sensitivity NT 0.8 | 0.769 | 0.969 | 0.912 |
| 81 | Specificity fix 0.9 | 0.490 | 0.969 | 0.471 |
| 81 | Specificity NT 0.85 | 0.402 | 0.969 | 0.353 |
| 81 | Youden | 0.591 | 0.969 | 0.618 |
| 82 | optim. group sep. | 0.541 | 0.962 | 0.529 |
| 82 | Sensitivity fix 0.85 | 0.631 | 0.962 | 0.706 |
| 82 | Sensitivity NT 0.8 | 0.728 | 0.962 | 0.824 |
| 82 | Specificity fix 0.9 | 0.514 | 0.962 | 0.500 |
| 82 | Specificity NT 0.85 | 0.435 | 0.962 | 0.382 |
| 82 | Youden | 0.658 | 0.962 | 0.735 |
| 83 | optim. group sep. | 0.538 | 0.968 | 0.529 |
| 83 | Sensitivity fix 0.85 | 0.681 | 0.968 | 0.706 |
| 83 | Sensitivity NT 0.8 | 0.784 | 0.968 | 0.853 |
| 83 | Specificity fix 0.9 | 0.476 | 0.968 | 0.441 |
| 83 | Specificity NT 0.85 | 0.422 | 0.968 | 0.324 |
| 83 | Youden | 0.572 | 0.968 | 0.559 |
| 84 | optim. group sep. | 0.541 | 0.966 | 0.618 |
| 84 | Sensitivity fix 0.85 | 0.641 | 0.966 | 0.735 |
| 84 | Sensitivity NT 0.8 | 0.722 | 0.966 | 0.882 |
| 84 | Specificity fix 0.9 | 0.513 | 0.966 | 0.559 |
| 84 | Specificity NT 0.85 | 0.419 | 0.966 | 0.412 |
| 84 | Youden | 0.641 | 0.966 | 0.735 |
| 85 | optim. group sep. | 0.538 | 0.969 | 0.529 |
| 85 | Sensitivity fix 0.85 | 0.690 | 0.969 | 0.765 |
| 85 | Sensitivity NT 0.8 | 0.780 | 0.969 | 0.853 |
| 85 | Specificity fix 0.9 | 0.485 | 0.969 | 0.441 |
| 85 | Specificity NT 0.85 | 0.412 | 0.969 | 0.382 |
| 85 | Youden | 0.587 | 0.969 | 0.559 |
| 86 | optim. group sep. | 0.541 | 0.962 | 0.529 |
| 86 | Sensitivity fix 0.85 | 0.631 | 0.962 | 0.706 |
| 86 | Sensitivity NT 0.8 | 0.728 | 0.962 | 0.824 |
| 86 | Specificity fix 0.9 | 0.514 | 0.962 | 0.500 |
| 86 | Specificity NT 0.85 | 0.435 | 0.962 | 0.382 |
| 86 | Youden | 0.658 | 0.962 | 0.735 |
| 87 | optim. group sep. | 0.539 | 0.967 | 0.559 |
| 87 | Sensitivity fix 0.85 | 0.654 | 0.967 | 0.676 |
| 87 | Sensitivity NT 0.8 | 0.768 | 0.967 | 0.853 |
| 87 | Specificity fix 0.9 | 0.489 | 0.967 | 0.500 |
| 87 | Specificity NT 0.85 | 0.413 | 0.967 | 0.353 |
| 87 | Youden | 0.570 | 0.967 | 0.559 |
| 88 | optim. group sep. | 0.539 | 0.967 | 0.559 |
| 88 | Sensitivity fix 0.85 | 0.660 | 0.967 | 0.735 |
| 88 | Sensitivity NT 0.8 | 0.766 | 0.967 | 0.853 |
| 88 | Specificity fix 0.9 | 0.491 | 0.967 | 0.471 |
| 88 | Specificity NT 0.85 | 0.410 | 0.967 | 0.353 |
| 88 | Youden | 0.572 | 0.967 | 0.559 |
| 89 | optim. group sep. | 0.539 | 0.967 | 0.471 |
| 89 | Sensitivity fix 0.85 | 0.665 | 0.967 | 0.706 |
| 89 | Sensitivity NT 0.8 | 0.774 | 0.967 | 0.824 |
| 89 | Specificity fix 0.9 | 0.496 | 0.967 | 0.471 |
| 89 | Specificity NT 0.85 | 0.428 | 0.967 | 0.382 |
| 89 | Youden | 0.597 | 0.967 | 0.588 |
| 90 | optim. group sep. | 0.539 | 0.967 | 0.559 |
| 90 | Sensitivity fix 0.85 | 0.660 | 0.967 | 0.735 |
| 90 | Sensitivity NT 0.8 | 0.766 | 0.967 | 0.853 |
| 90 | Specificity fix 0.9 | 0.491 | 0.967 | 0.471 |
| 90 | Specificity NT 0.85 | 0.410 | 0.967 | 0.353 |
| 90 | Youden | 0.572 | 0.967 | 0.559 |
| 91 | optim. group sep. | 0.539 | 0.966 | 0.471 |
| 91 | Sensitivity fix 0.85 | 0.663 | 0.966 | 0.618 |
| 91 | Sensitivity NT 0.8 | 0.762 | 0.966 | 0.824 |
| 91 | Specificity fix 0.9 | 0.493 | 0.966 | 0.441 |
| 91 | Specificity NT 0.85 | 0.412 | 0.966 | 0.294 |
| 91 | Youden | 0.589 | 0.966 | 0.559 |
| 92 | optim. group sep. | 0.540 | 0.967 | 0.559 |
| 92 | Sensitivity fix 0.85 | 0.658 | 0.967 | 0.735 |
| 92 | Sensitivity NT 0.8 | 0.760 | 0.967 | 0.853 |
| 92 | Specificity fix 0.9 | 0.517 | 0.967 | 0.529 |
| 92 | Specificity NT 0.85 | 0.421 | 0.967 | 0.382 |
| 92 | Youden | 0.575 | 0.967 | 0.559 |
| 93 | optim. group sep. | 0.538 | 0.967 | 0.500 |
| 93 | Sensitivity fix 0.85 | 0.682 | 0.967 | 0.618 |
| 93 | Sensitivity NT 0.8 | 0.770 | 0.967 | 0.824 |
| 93 | Specificity fix 0.9 | 0.508 | 0.967 | 0.441 |
| 93 | Specificity NT 0.85 | 0.421 | 0.967 | 0.324 |
| 93 | Youden | 0.568 | 0.967 | 0.500 |
| 94 | optim. group sep. | 0.541 | 0.964 | 0.618 |
| 94 | Sensitivity fix 0.85 | 0.651 | 0.964 | 0.706 |
| 94 | Sensitivity NT 0.8 | 0.747 | 0.964 | 0.824 |
| 94 | Specificity fix 0.9 | 0.519 | 0.964 | 0.559 |
| 94 | Specificity NT 0.85 | 0.448 | 0.964 | 0.471 |
| 94 | Youden | 0.651 | 0.964 | 0.706 |
| 95 | optim. group sep. | 0.539 | 0.967 | 0.500 |
| 95 | Sensitivity fix 0.85 | 0.690 | 0.967 | 0.735 |
| 95 | Sensitivity NT 0.8 | 0.782 | 0.967 | 0.824 |
| 95 | Specificity fix 0.9 | 0.500 | 0.967 | 0.471 |
| 95 | Specificity NT 0.85 | 0.430 | 0.967 | 0.324 |
| 95 | Youden | 0.571 | 0.967 | 0.529 |
| 96 | optim. group sep. | 0.541 | 0.965 | 0.647 |
| 96 | Sensitivity fix 0.85 | 0.650 | 0.965 | 0.824 |
| 96 | Sensitivity NT 0.8 | 0.722 | 0.965 | 0.853 |
| 96 | Specificity fix 0.9 | 0.522 | 0.965 | 0.618 |
| 96 | Specificity NT 0.85 | 0.446 | 0.965 | 0.500 |
| 96 | Youden | 0.599 | 0.965 | 0.765 |
| 97 | optim. group sep. | 0.538 | 0.968 | 0.471 |
| 97 | Sensitivity fix 0.85 | 0.679 | 0.968 | 0.706 |
| 97 | Sensitivity NT 0.8 | 0.785 | 0.968 | 0.853 |
| 97 | Specificity fix 0.9 | 0.480 | 0.968 | 0.441 |
| 97 | Specificity NT 0.85 | 0.417 | 0.968 | 0.324 |
| 97 | Youden | 0.572 | 0.968 | 0.529 |
| 98 | optim. group sep. | 0.539 | 0.967 | 0.471 |
| 98 | Sensitivity fix 0.85 | 0.664 | 0.967 | 0.618 |
| 98 | Sensitivity NT 0.8 | 0.760 | 0.967 | 0.824 |
| 98 | Specificity fix 0.9 | 0.478 | 0.967 | 0.382 |
| 98 | Specificity NT 0.85 | 0.424 | 0.967 | 0.382 |
| 98 | Youden | 0.522 | 0.967 | 0.441 |
| 99 | optim. group sep. | 0.539 | 0.967 | 0.500 |
| 99 | Sensitivity fix 0.85 | 0.673 | 0.967 | 0.618 |
| 99 | Sensitivity NT 0.8 | 0.769 | 0.967 | 0.824 |
| 99 | Specificity fix 0.9 | 0.501 | 0.967 | 0.441 |
| 99 | Specificity NT 0.85 | 0.422 | 0.967 | 0.324 |
| 99 | Youden | 0.577 | 0.967 | 0.500 |
| 100 | optim. group sep. | 0.539 | 0.967 | 0.559 |
| 100 | Sensitivity fix 0.85 | 0.660 | 0.967 | 0.735 |
| 100 | Sensitivity NT 0.8 | 0.766 | 0.967 | 0.853 |
| 100 | Specificity fix 0.9 | 0.491 | 0.967 | 0.471 |
| 100 | Specificity NT 0.85 | 0.410 | 0.967 | 0.353 |
| 100 | Youden | 0.572 | 0.967 | 0.559 |
| 101 | optim. group sep. | 0.538 | 0.968 | 0.471 |
| 101 | Sensitivity fix 0.85 | 0.683 | 0.968 | 0.676 |
| 101 | Sensitivity NT 0.8 | 0.782 | 0.968 | 0.824 |
| 101 | Specificity fix 0.9 | 0.482 | 0.968 | 0.441 |
| 101 | Specificity NT 0.85 | 0.420 | 0.968 | 0.353 |
| 101 | Youden | 0.586 | 0.968 | 0.559 |
| 102 | optim. group sep. | 0.540 | 0.966 | 0.441 |
| 102 | Sensitivity fix 0.85 | 0.634 | 0.966 | 0.529 |
| 102 | Sensitivity NT 0.8 | 0.763 | 0.966 | 0.735 |
| 102 | Specificity fix 0.9 | 0.498 | 0.966 | 0.382 |
| 102 | Specificity NT 0.85 | 0.438 | 0.966 | 0.265 |
| 102 | Youden | 0.630 | 0.966 | 0.529 |
| 103 | optim. group sep. | 0.545 | 0.955 | 0.618 |
| 103 | Sensitivity fix 0.85 | 0.590 | 0.955 | 0.647 |
| 103 | Sensitivity NT 0.8 | 0.674 | 0.955 | 0.676 |
| 103 | Specificity fix 0.9 | 0.541 | 0.955 | 0.618 |
| 103 | Specificity NT 0.85 | 0.505 | 0.955 | 0.559 |
| 103 | Youden | 0.590 | 0.955 | 0.647 |
| 104 | optim. group sep. | 0.541 | 0.967 | 0.471 |
| 104 | Sensitivity fix 0.85 | 0.674 | 0.967 | 0.706 |
| 104 | Sensitivity NT 0.8 | 0.750 | 0.967 | 0.824 |
| 104 | Specificity fix 0.9 | 0.504 | 0.967 | 0.471 |
| 104 | Specificity NT 0.85 | 0.452 | 0.967 | 0.353 |
| 104 | Youden | 0.605 | 0.967 | 0.618 |
| 105 | optim. group sep. | 0.539 | 0.967 | 0.529 |
| 105 | Sensitivity fix 0.85 | 0.655 | 0.967 | 0.765 |
| 105 | Sensitivity NT 0.8 | 0.768 | 0.967 | 0.824 |
| 105 | Specificity fix 0.9 | 0.490 | 0.967 | 0.441 |
| 105 | Specificity NT 0.85 | 0.423 | 0.967 | 0.324 |
| 105 | Youden | 0.591 | 0.967 | 0.618 |
| 106 | optim. group sep. | 0.541 | 0.962 | 0.471 |
| 106 | Sensitivity fix 0.85 | 0.611 | 0.962 | 0.618 |
| 106 | Sensitivity NT 0.8 | 0.741 | 0.962 | 0.765 |
| 106 | Specificity fix 0.9 | 0.540 | 0.962 | 0.471 |
| 106 | Specificity NT 0.85 | 0.445 | 0.962 | 0.235 |
| 106 | Youden | 0.649 | 0.962 | 0.676 |
| 107 | optim. group sep. | 0.541 | 0.965 | 0.382 |
| 107 | Sensitivity fix 0.85 | 0.664 | 0.965 | 0.588 |
| 107 | Sensitivity NT 0.8 | 0.748 | 0.965 | 0.735 |
| 107 | Specificity fix 0.9 | 0.522 | 0.965 | 0.353 |
| 107 | Specificity NT 0.85 | 0.430 | 0.965 | 0.235 |
| 107 | Youden | 0.586 | 0.965 | 0.471 |
| 108 | optim. group sep. | 0.542 | 0.962 | 0.412 |
| 108 | Sensitivity fix 0.85 | 0.630 | 0.962 | 0.588 |
| 108 | Sensitivity NT 0.8 | 0.731 | 0.962 | 0.765 |
| 108 | Specificity fix 0.9 | 0.528 | 0.962 | 0.353 |
| 108 | Specificity NT 0.85 | 0.458 | 0.962 | 0.294 |
| 108 | Youden | 0.656 | 0.962 | 0.676 |
| 109 | optim. group sep. | 0.541 | 0.962 | 0.559 |
| 109 | Sensitivity fix 0.85 | 0.623 | 0.962 | 0.676 |
| 109 | Sensitivity NT 0.8 | 0.746 | 0.962 | 0.824 |
| 109 | Specificity fix 0.9 | 0.490 | 0.962 | 0.441 |
| 109 | Specificity NT 0.85 | 0.449 | 0.962 | 0.412 |
| 109 | Youden | 0.535 | 0.962 | 0.529 |
| 110 | optim. group sep. | 0.543 | 0.959 | 0.618 |
| 110 | Sensitivity fix 0.85 | 0.613 | 0.959 | 0.676 |
| 110 | Sensitivity NT 0.8 | 0.729 | 0.959 | 0.794 |
| 110 | Specificity fix 0.9 | 0.539 | 0.959 | 0.618 |
| 110 | Specificity NT 0.85 | 0.451 | 0.959 | 0.441 |
| 110 | Youden | 0.635 | 0.959 | 0.735 |
| 111 | optim. group sep. | 0.542 | 0.962 | 0.500 |
| 111 | Sensitivity fix 0.85 | 0.644 | 0.962 | 0.676 |
| 111 | Sensitivity NT 0.8 | 0.739 | 0.962 | 0.824 |
| 111 | Specificity fix 0.9 | 0.500 | 0.962 | 0.441 |
| 111 | Specificity NT 0.85 | 0.459 | 0.962 | 0.441 |
| 111 | Youden | 0.518 | 0.962 | 0.500 |
| 112 | optim. group sep. | 0.544 | 0.959 | 0.588 |
| 112 | Sensitivity fix 0.85 | 0.614 | 0.959 | 0.676 |
| 112 | Sensitivity NT 0.8 | 0.700 | 0.959 | 0.824 |
| 112 | Specificity fix 0.9 | 0.557 | 0.959 | 0.588 |
| 112 | Specificity NT 0.85 | 0.466 | 0.959 | 0.412 |
| 112 | Youden | 0.572 | 0.959 | 0.588 |
| 113 | optim. group sep. | 0.541 | 0.961 | 0.441 |
| 113 | Sensitivity fix 0.85 | 0.640 | 0.961 | 0.588 |
| 113 | Sensitivity NT 0.8 | 0.734 | 0.961 | 0.765 |
| 113 | Specificity fix 0.9 | 0.512 | 0.961 | 0.412 |
| 113 | Specificity NT 0.85 | 0.442 | 0.961 | 0.265 |
| 113 | Youden | 0.549 | 0.961 | 0.441 |
| 114 | optim. group sep. | 0.543 | 0.958 | 0.471 |
| 114 | Sensitivity fix 0.85 | 0.606 | 0.958 | 0.706 |
| 114 | Sensitivity NT 0.8 | 0.723 | 0.958 | 0.765 |
| 114 | Specificity fix 0.9 | 0.535 | 0.958 | 0.441 |
| 114 | Specificity NT 0.85 | 0.457 | 0.958 | 0.353 |
| 114 | Youden | 0.638 | 0.958 | 0.735 |
| 115 | optim. group sep. | 0.543 | 0.960 | 0.353 |
| 115 | Sensitivity fix 0.85 | 0.630 | 0.960 | 0.559 |
| 115 | Sensitivity NT 0.8 | 0.719 | 0.960 | 0.735 |
| 115 | Specificity fix 0.9 | 0.500 | 0.960 | 0.324 |
| 115 | Specificity NT 0.85 | 0.451 | 0.960 | 0.265 |
| 115 | Youden | 0.657 | 0.960 | 0.647 |
| 116 | optim. group sep. | 0.541 | 0.965 | 0.618 |
| 116 | Sensitivity fix 0.85 | 0.629 | 0.965 | 0.706 |
| 116 | Sensitivity NT 0.8 | 0.739 | 0.965 | 0.824 |
| 116 | Specificity fix 0.9 | 0.521 | 0.965 | 0.588 |
| 116 | Specificity NT 0.85 | 0.433 | 0.965 | 0.353 |
| 116 | Youden | 0.636 | 0.965 | 0.706 |
| 117 | optim. group sep. | 0.544 | 0.957 | 0.412 |
| 117 | Sensitivity fix 0.85 | 0.626 | 0.957 | 0.647 |
| 117 | Sensitivity NT 0.8 | 0.689 | 0.957 | 0.765 |
| 117 | Specificity fix 0.9 | 0.524 | 0.957 | 0.382 |
| 117 | Specificity NT 0.85 | 0.478 | 0.957 | 0.353 |
| 117 | Youden | 0.527 | 0.957 | 0.412 |
| 118 | optim. group sep. | 0.540 | 0.967 | 0.471 |
| 118 | Sensitivity fix 0.85 | 0.668 | 0.967 | 0.706 |
| 118 | Sensitivity NT 0.8 | 0.747 | 0.967 | 0.824 |
| 118 | Specificity fix 0.9 | 0.497 | 0.967 | 0.441 |
| 118 | Specificity NT 0.85 | 0.453 | 0.967 | 0.353 |
| 118 | Youden | 0.596 | 0.967 | 0.618 |
| 119 | optim. group sep. | 0.542 | 0.964 | 0.559 |
| 119 | Sensitivity fix 0.85 | 0.658 | 0.964 | 0.735 |
| 119 | Sensitivity NT 0.8 | 0.727 | 0.964 | 0.794 |
| 119 | Specificity fix 0.9 | 0.546 | 0.964 | 0.588 |
| 119 | Specificity NT 0.85 | 0.450 | 0.964 | 0.353 |
| 119 | Youden | 0.637 | 0.964 | 0.676 |
| 120 | optim. group sep. | 0.540 | 0.963 | 0.441 |
| 120 | Sensitivity fix 0.85 | 0.636 | 0.963 | 0.529 |
| 120 | Sensitivity NT 0.8 | 0.744 | 0.963 | 0.735 |
| 120 | Specificity fix 0.9 | 0.483 | 0.963 | 0.353 |
| 120 | Specificity NT 0.85 | 0.445 | 0.963 | 0.294 |
| 120 | Youden | 0.611 | 0.963 | 0.529 |
| 121 | optim. group sep. | 0.542 | 0.960 | 0.441 |
| 121 | Sensitivity fix 0.85 | 0.613 | 0.960 | 0.647 |
| 121 | Sensitivity NT 0.8 | 0.733 | 0.960 | 0.735 |
| 121 | Specificity fix 0.9 | 0.512 | 0.960 | 0.441 |
| 121 | Specificity NT 0.85 | 0.441 | 0.960 | 0.235 |
| 121 | Youden | 0.501 | 0.960 | 0.412 |
| 122 | optim. group sep. | 0.541 | 0.963 | 0.324 |
| 122 | Sensitivity fix 0.85 | 0.658 | 0.963 | 0.588 |
| 122 | Sensitivity NT 0.8 | 0.736 | 0.963 | 0.735 |
| 122 | Specificity fix 0.9 | 0.505 | 0.963 | 0.294 |
| 122 | Specificity NT 0.85 | 0.452 | 0.963 | 0.265 |
| 122 | Youden | 0.620 | 0.963 | 0.529 |
| 123 | optim. group sep. | 0.543 | 0.960 | 0.382 |
| 123 | Sensitivity fix 0.85 | 0.632 | 0.960 | 0.647 |
| 123 | Sensitivity NT 0.8 | 0.712 | 0.960 | 0.765 |
| 123 | Specificity fix 0.9 | 0.530 | 0.960 | 0.353 |
| 123 | Specificity NT 0.85 | 0.463 | 0.960 | 0.235 |
| 123 | Youden | 0.632 | 0.960 | 0.647 |
| 124 | optim. group sep. | 0.540 | 0.965 | 0.471 |
| 124 | Sensitivity fix 0.85 | 0.637 | 0.965 | 0.529 |
| 124 | Sensitivity NT 0.8 | 0.755 | 0.965 | 0.765 |
| 124 | Specificity fix 0.9 | 0.508 | 0.965 | 0.412 |
| 124 | Specificity NT 0.85 | 0.429 | 0.965 | 0.206 |
| 124 | Youden | 0.607 | 0.965 | 0.529 |
| 125 | optim. group sep. | 0.539 | 0.968 | 0.559 |
| 125 | Sensitivity fix 0.85 | 0.679 | 0.968 | 0.706 |
| 125 | Sensitivity NT 0.8 | 0.772 | 0.968 | 0.794 |
| 125 | Specificity fix 0.9 | 0.503 | 0.968 | 0.471 |
| 125 | Specificity NT 0.85 | 0.437 | 0.968 | 0.353 |
| 125 | Youden | 0.576 | 0.968 | 0.559 |
| 126 | optim. group sep. | 0.539 | 0.968 | 0.559 |
| 126 | Sensitivity fix 0.85 | 0.675 | 0.968 | 0.735 |
| 126 | Sensitivity NT 0.8 | 0.774 | 0.968 | 0.794 |
| 126 | Specificity fix 0.9 | 0.500 | 0.968 | 0.529 |
| 126 | Specificity NT 0.85 | 0.428 | 0.968 | 0.382 |
| 126 | Youden | 0.583 | 0.968 | 0.588 |
| 127 | optim. group sep. | 0.540 | 0.966 | 0.500 |
| 127 | Sensitivity fix 0.85 | 0.671 | 0.966 | 0.706 |
| 127 | Sensitivity NT 0.8 | 0.760 | 0.966 | 0.824 |
| 127 | Specificity fix 0.9 | 0.497 | 0.966 | 0.441 |
| 127 | Specificity NT 0.85 | 0.437 | 0.966 | 0.324 |
| 127 | Youden | 0.583 | 0.966 | 0.500 |
| 128 | optim. group sep. | 0.540 | 0.967 | 0.559 |
| 128 | Sensitivity fix 0.85 | 0.664 | 0.967 | 0.706 |
| 128 | Sensitivity NT 0.8 | 0.767 | 0.967 | 0.853 |
| 128 | Specificity fix 0.9 | 0.496 | 0.967 | 0.441 |
| 128 | Specificity NT 0.85 | 0.430 | 0.967 | 0.382 |
| 128 | Youden | 0.581 | 0.967 | 0.559 |
| 129 | optim. group sep. | 0.539 | 0.968 | 0.529 |
| 129 | Sensitivity fix 0.85 | 0.689 | 0.968 | 0.765 |
| 129 | Sensitivity NT 0.8 | 0.770 | 0.968 | 0.824 |
| 129 | Specificity fix 0.9 | 0.491 | 0.968 | 0.441 |
| 129 | Specificity NT 0.85 | 0.427 | 0.968 | 0.353 |
| 129 | Youden | 0.620 | 0.968 | 0.618 |
| 130 | optim. group sep. | 0.539 | 0.969 | 0.529 |
| 130 | Sensitivity fix 0.85 | 0.690 | 0.969 | 0.765 |
| 130 | Sensitivity NT 0.8 | 0.760 | 0.969 | 0.853 |
| 130 | Specificity fix 0.9 | 0.496 | 0.969 | 0.471 |
| 130 | Specificity NT 0.85 | 0.416 | 0.969 | 0.353 |
| 130 | Youden | 0.568 | 0.969 | 0.647 |
| 131 | optim. group sep. | 0.539 | 0.967 | 0.500 |
| 131 | Sensitivity fix 0.85 | 0.677 | 0.967 | 0.735 |
| 131 | Sensitivity NT 0.8 | 0.774 | 0.967 | 0.824 |
| 131 | Specificity fix 0.9 | 0.478 | 0.967 | 0.441 |
| 131 | Specificity NT 0.85 | 0.420 | 0.967 | 0.324 |
| 131 | Youden | 0.569 | 0.967 | 0.529 |
| 132 | optim. group sep. | 0.540 | 0.968 | 0.559 |
| 132 | Sensitivity fix 0.85 | 0.660 | 0.968 | 0.735 |
| 132 | Sensitivity NT 0.8 | 0.766 | 0.968 | 0.853 |
| 132 | Specificity fix 0.9 | 0.491 | 0.968 | 0.471 |
| 132 | Specificity NT 0.85 | 0.410 | 0.968 | 0.353 |
| 132 | Youden | 0.572 | 0.968 | 0.559 |
| 133 | optim. group sep. | 0.539 | 0.968 | 0.529 |
| 133 | Sensitivity fix 0.85 | 0.676 | 0.968 | 0.706 |
| 133 | Sensitivity NT 0.8 | 0.780 | 0.968 | 0.824 |
| 133 | Specificity fix 0.9 | 0.505 | 0.968 | 0.471 |
| 133 | Specificity NT 0.85 | 0.426 | 0.968 | 0.382 |
| 133 | Youden | 0.586 | 0.968 | 0.559 |
| 134 | optim. group sep. | 0.540 | 0.965 | 0.500 |
| 134 | Sensitivity fix 0.85 | 0.674 | 0.965 | 0.676 |
| 134 | Sensitivity NT 0.8 | 0.761 | 0.965 | 0.824 |
| 134 | Specificity fix 0.9 | 0.500 | 0.965 | 0.382 |
| 134 | Specificity NT 0.85 | 0.447 | 0.965 | 0.324 |
| 134 | Youden | 0.594 | 0.965 | 0.500 |
| 135 | optim. group sep. | 0.540 | 0.966 | 0.500 |
| 135 | Sensitivity fix 0.85 | 0.670 | 0.966 | 0.706 |
| 135 | Sensitivity NT 0.8 | 0.762 | 0.966 | 0.824 |
| 135 | Specificity fix 0.9 | 0.500 | 0.966 | 0.412 |
| 135 | Specificity NT 0.85 | 0.445 | 0.966 | 0.382 |
| 135 | Youden | 0.611 | 0.966 | 0.588 |
| 136 | optim. group sep. | 0.538 | 0.968 | 0.529 |
| 136 | Sensitivity fix 0.85 | 0.686 | 0.968 | 0.735 |
| 136 | Sensitivity NT 0.8 | 0.775 | 0.968 | 0.824 |
| 136 | Specificity fix 0.9 | 0.492 | 0.968 | 0.412 |
| 136 | Specificity NT 0.85 | 0.436 | 0.968 | 0.353 |
| 136 | Youden | 0.649 | 0.968 | 0.618 |
| 137 | optim. group sep. | 0.539 | 0.968 | 0.529 |
| 137 | Sensitivity fix 0.85 | 0.694 | 0.968 | 0.765 |
| 137 | Sensitivity NT 0.8 | 0.766 | 0.968 | 0.853 |
| 137 | Specificity fix 0.9 | 0.494 | 0.968 | 0.412 |
| 137 | Specificity NT 0.85 | 0.423 | 0.968 | 0.353 |
| 137 | Youden | 0.556 | 0.968 | 0.529 |
| 138 | optim. group sep. | 0.539 | 0.966 | 0.471 |
| 138 | Sensitivity fix 0.85 | 0.676 | 0.966 | 0.735 |
| 138 | Sensitivity NT 0.8 | 0.778 | 0.966 | 0.824 |
| 138 | Specificity fix 0.9 | 0.492 | 0.966 | 0.412 |
| 138 | Specificity NT 0.85 | 0.416 | 0.966 | 0.324 |
| 138 | Youden | 0.575 | 0.966 | 0.529 |
| 139 | optim. group sep. | 0.540 | 0.967 | 0.500 |
| 139 | Sensitivity fix 0.85 | 0.651 | 0.967 | 0.706 |
| 139 | Sensitivity NT 0.8 | 0.769 | 0.967 | 0.853 |
| 139 | Specificity fix 0.9 | 0.493 | 0.967 | 0.471 |
| 139 | Specificity NT 0.85 | 0.413 | 0.967 | 0.353 |
| 139 | Youden | 0.574 | 0.967 | 0.559 |
| 140 | optim. group sep. | 0.539 | 0.968 | 0.500 |
| 140 | Sensitivity fix 0.85 | 0.676 | 0.968 | 0.647 |
| 140 | Sensitivity NT 0.8 | 0.767 | 0.968 | 0.824 |
| 140 | Specificity fix 0.9 | 0.507 | 0.968 | 0.412 |
| 140 | Specificity NT 0.85 | 0.438 | 0.968 | 0.324 |
| 140 | Youden | 0.582 | 0.968 | 0.529 |
| 141 | optim. group sep. | 0.539 | 0.968 | 0.500 |
| 141 | Sensitivity fix 0.85 | 0.680 | 0.968 | 0.676 |
| 141 | Sensitivity NT 0.8 | 0.773 | 0.968 | 0.824 |
| 141 | Specificity fix 0.9 | 0.509 | 0.968 | 0.441 |
| 141 | Specificity NT 0.85 | 0.423 | 0.968 | 0.353 |
| 141 | Youden | 0.599 | 0.968 | 0.529 |
| 142 | optim. group sep. | 0.540 | 0.966 | 0.471 |
| 142 | Sensitivity fix 0.85 | 0.680 | 0.966 | 0.647 |
| 142 | Sensitivity NT 0.8 | 0.758 | 0.966 | 0.794 |
| 142 | Specificity fix 0.9 | 0.504 | 0.966 | 0.382 |
| 142 | Specificity NT 0.85 | 0.433 | 0.966 | 0.324 |
| 142 | Youden | 0.582 | 0.966 | 0.500 |
| 143 | optim. group sep. | 0.540 | 0.966 | 0.500 |
| 143 | Sensitivity fix 0.85 | 0.670 | 0.966 | 0.647 |
| 143 | Sensitivity NT 0.8 | 0.758 | 0.966 | 0.824 |
| 143 | Specificity fix 0.9 | 0.512 | 0.966 | 0.471 |
| 143 | Specificity NT 0.85 | 0.424 | 0.966 | 0.324 |
| 143 | Youden | 0.599 | 0.966 | 0.559 |
| 144 | optim. group sep. | 0.539 | 0.968 | 0.471 |
| 144 | Sensitivity fix 0.85 | 0.689 | 0.968 | 0.706 |
| 144 | Sensitivity NT 0.8 | 0.774 | 0.968 | 0.853 |
| 144 | Specificity fix 0.9 | 0.485 | 0.968 | 0.382 |
| 144 | Specificity NT 0.85 | 0.411 | 0.968 | 0.294 |
| 144 | Youden | 0.633 | 0.968 | 0.618 |
| 145 | optim. group sep. | 0.540 | 0.969 | 0.500 |
| 145 | Sensitivity fix 0.85 | 0.681 | 0.969 | 0.765 |
| 145 | Sensitivity NT 0.8 | 0.755 | 0.969 | 0.853 |
| 145 | Specificity fix 0.9 | 0.488 | 0.969 | 0.412 |
| 145 | Specificity NT 0.85 | 0.414 | 0.969 | 0.353 |
| 145 | Youden | 0.630 | 0.969 | 0.676 |
| 146 | optim. group sep. | 0.539 | 0.966 | 0.412 |
| 146 | Sensitivity fix 0.85 | 0.669 | 0.966 | 0.647 |
| 146 | Sensitivity NT 0.8 | 0.765 | 0.966 | 0.794 |
| 146 | Specificity fix 0.9 | 0.488 | 0.966 | 0.382 |
| 146 | Specificity NT 0.85 | 0.416 | 0.966 | 0.324 |
| 146 | Youden | 0.578 | 0.966 | 0.559 |
| 147 | optim. group sep. | 0.540 | 0.967 | 0.471 |
| 147 | Sensitivity fix 0.85 | 0.671 | 0.967 | 0.676 |
| 147 | Sensitivity NT 0.8 | 0.757 | 0.967 | 0.853 |
| 147 | Specificity fix 0.9 | 0.496 | 0.967 | 0.441 |
| 147 | Specificity NT 0.85 | 0.418 | 0.967 | 0.353 |
| 147 | Youden | 0.595 | 0.967 | 0.559 |
| 148 | optim. group sep. | 0.539 | 0.968 | 0.559 |
| 148 | Sensitivity fix 0.85 | 0.682 | 0.968 | 0.735 |
| 148 | Sensitivity NT 0.8 | 0.778 | 0.968 | 0.824 |
| 148 | Specificity fix 0.9 | 0.503 | 0.968 | 0.471 |
| 148 | Specificity NT 0.85 | 0.438 | 0.968 | 0.353 |
| 148 | Youden | 0.595 | 0.968 | 0.559 |
| 149 | optim. group sep. | 0.539 | 0.968 | 0.559 |
| 149 | Sensitivity fix 0.85 | 0.678 | 0.968 | 0.735 |
| 149 | Sensitivity NT 0.8 | 0.779 | 0.968 | 0.794 |
| 149 | Specificity fix 0.9 | 0.506 | 0.968 | 0.500 |
| 149 | Specificity NT 0.85 | 0.433 | 0.968 | 0.382 |
| 149 | Youden | 0.592 | 0.968 | 0.588 |
| 150 | optim. group sep. | 0.540 | 0.966 | 0.500 |
| 150 | Sensitivity fix 0.85 | 0.677 | 0.966 | 0.735 |
| 150 | Sensitivity NT 0.8 | 0.766 | 0.966 | 0.824 |
| 150 | Specificity fix 0.9 | 0.499 | 0.966 | 0.471 |
| 150 | Specificity NT 0.85 | 0.455 | 0.966 | 0.353 |
| 150 | Youden | 0.603 | 0.966 | 0.559 |
| 151 | optim. group sep. | 0.540 | 0.966 | 0.559 |
| 151 | Sensitivity fix 0.85 | 0.670 | 0.966 | 0.706 |
| 151 | Sensitivity NT 0.8 | 0.767 | 0.966 | 0.824 |
| 151 | Specificity fix 0.9 | 0.501 | 0.966 | 0.471 |
| 151 | Specificity NT 0.85 | 0.439 | 0.966 | 0.382 |
| 151 | Youden | 0.616 | 0.966 | 0.647 |
| 152 | optim. group sep. | 0.539 | 0.968 | 0.529 |
| 152 | Sensitivity fix 0.85 | 0.695 | 0.968 | 0.794 |
| 152 | Sensitivity NT 0.8 | 0.772 | 0.968 | 0.824 |
| 152 | Specificity fix 0.9 | 0.492 | 0.968 | 0.441 |
| 152 | Specificity NT 0.85 | 0.426 | 0.968 | 0.353 |
| 152 | Youden | 0.638 | 0.968 | 0.647 |
| 153 | optim. group sep. | 0.539 | 0.968 | 0.529 |
| 153 | Sensitivity fix 0.85 | 0.691 | 0.968 | 0.765 |
| 153 | Sensitivity NT 0.8 | 0.764 | 0.968 | 0.853 |
| 153 | Specificity fix 0.9 | 0.485 | 0.968 | 0.471 |
| 153 | Specificity NT 0.85 | 0.424 | 0.968 | 0.353 |
| 153 | Youden | 0.574 | 0.968 | 0.647 |
| 154 | optim. group sep. | 0.539 | 0.966 | 0.500 |
| 154 | Sensitivity fix 0.85 | 0.668 | 0.966 | 0.735 |
| 154 | Sensitivity NT 0.8 | 0.774 | 0.966 | 0.824 |
| 154 | Specificity fix 0.9 | 0.478 | 0.966 | 0.471 |
| 154 | Specificity NT 0.85 | 0.421 | 0.966 | 0.324 |
| 154 | Youden | 0.569 | 0.966 | 0.529 |
| 155 | optim. group sep. | 0.540 | 0.967 | 0.559 |
| 155 | Sensitivity fix 0.85 | 0.661 | 0.967 | 0.735 |
| 155 | Sensitivity NT 0.8 | 0.775 | 0.967 | 0.853 |
| 155 | Specificity fix 0.9 | 0.489 | 0.967 | 0.500 |
| 155 | Specificity NT 0.85 | 0.413 | 0.967 | 0.353 |
| 155 | Youden | 0.570 | 0.967 | 0.559 |
| 156 | optim. group sep. | 0.539 | 0.968 | 0.559 |
| 156 | Sensitivity fix 0.85 | 0.679 | 0.968 | 0.676 |
| 156 | Sensitivity NT 0.8 | 0.772 | 0.968 | 0.794 |
| 156 | Specificity fix 0.9 | 0.502 | 0.968 | 0.471 |
| 156 | Specificity NT 0.85 | 0.441 | 0.968 | 0.353 |
| 156 | Youden | 0.578 | 0.968 | 0.559 |
| 157 | optim. group sep. | 0.539 | 0.968 | 0.529 |
| 157 | Sensitivity fix 0.85 | 0.675 | 0.968 | 0.735 |
| 157 | Sensitivity NT 0.8 | 0.779 | 0.968 | 0.824 |
| 157 | Specificity fix 0.9 | 0.507 | 0.968 | 0.500 |
| 157 | Specificity NT 0.85 | 0.428 | 0.968 | 0.382 |
| 157 | Youden | 0.580 | 0.968 | 0.588 |
| 158 | optim. group sep. | 0.540 | 0.966 | 0.500 |
| 158 | Sensitivity fix 0.85 | 0.674 | 0.966 | 0.676 |
| 158 | Sensitivity NT 0.8 | 0.760 | 0.966 | 0.824 |
| 158 | Specificity fix 0.9 | 0.500 | 0.966 | 0.441 |
| 158 | Specificity NT 0.85 | 0.447 | 0.966 | 0.324 |
| 158 | Youden | 0.597 | 0.966 | 0.500 |
| 159 | optim. group sep. | 0.540 | 0.967 | 0.559 |
| 159 | Sensitivity fix 0.85 | 0.664 | 0.967 | 0.706 |
| 159 | Sensitivity NT 0.8 | 0.765 | 0.967 | 0.853 |
| 159 | Specificity fix 0.9 | 0.497 | 0.967 | 0.412 |
| 159 | Specificity NT 0.85 | 0.445 | 0.967 | 0.412 |
| 159 | Youden | 0.580 | 0.967 | 0.559 |
| 160 | optim. group sep. | 0.539 | 0.968 | 0.529 |
| 160 | Sensitivity fix 0.85 | 0.689 | 0.968 | 0.765 |
| 160 | Sensitivity NT 0.8 | 0.773 | 0.968 | 0.824 |
| 160 | Specificity fix 0.9 | 0.491 | 0.968 | 0.441 |
| 160 | Specificity NT 0.85 | 0.429 | 0.968 | 0.353 |
| 160 | Youden | 0.625 | 0.968 | 0.618 |
| 161 | optim. group sep. | 0.539 | 0.968 | 0.529 |
| 161 | Sensitivity fix 0.85 | 0.694 | 0.968 | 0.765 |
| 161 | Sensitivity NT 0.8 | 0.756 | 0.968 | 0.853 |
| 161 | Specificity fix 0.9 | 0.496 | 0.968 | 0.441 |
| 161 | Specificity NT 0.85 | 0.417 | 0.968 | 0.353 |
| 161 | Youden | 0.562 | 0.968 | 0.618 |
| 162 | optim. group sep. | 0.539 | 0.966 | 0.500 |
| 162 | Sensitivity fix 0.85 | 0.674 | 0.966 | 0.735 |
| 162 | Sensitivity NT 0.8 | 0.781 | 0.966 | 0.824 |
| 162 | Specificity fix 0.9 | 0.477 | 0.966 | 0.441 |
| 162 | Specificity NT 0.85 | 0.417 | 0.966 | 0.324 |
| 162 | Youden | 0.571 | 0.966 | 0.529 |
| 163 | optim. group sep. | 0.540 | 0.967 | 0.529 |
| 163 | Sensitivity fix 0.85 | 0.658 | 0.967 | 0.735 |
| 163 | Sensitivity NT 0.8 | 0.770 | 0.967 | 0.853 |
| 163 | Specificity fix 0.9 | 0.489 | 0.967 | 0.471 |
| 163 | Specificity NT 0.85 | 0.416 | 0.967 | 0.353 |
| 163 | Youden | 0.575 | 0.967 | 0.559 |
| 164 | optim. group sep. | 0.539 | 0.967 | 0.529 |
| 164 | Sensitivity fix 0.85 | 0.680 | 0.967 | 0.676 |
| 164 | Sensitivity NT 0.8 | 0.771 | 0.967 | 0.794 |
| 164 | Specificity fix 0.9 | 0.504 | 0.967 | 0.412 |
| 164 | Specificity NT 0.85 | 0.433 | 0.967 | 0.353 |
| 164 | Youden | 0.572 | 0.967 | 0.559 |
| 165 | optim. group sep. | 0.541 | 0.964 | 0.441 |
| 165 | Sensitivity fix 0.85 | 0.635 | 0.964 | 0.529 |
| 165 | Sensitivity NT 0.8 | 0.737 | 0.964 | 0.706 |
| 165 | Specificity fix 0.9 | 0.513 | 0.964 | 0.412 |
| 165 | Specificity NT 0.85 | 0.446 | 0.964 | 0.324 |
| 165 | Youden | 0.526 | 0.964 | 0.441 |
| 166 | optim. group sep. | 0.541 | 0.962 | 0.324 |
| 166 | Sensitivity fix 0.85 | 0.656 | 0.962 | 0.588 |
| 166 | Sensitivity NT 0.8 | 0.736 | 0.962 | 0.735 |
| 166 | Specificity fix 0.9 | 0.517 | 0.962 | 0.324 |
| 166 | Specificity NT 0.85 | 0.443 | 0.962 | 0.265 |
| 166 | Youden | 0.608 | 0.962 | 0.500 |
| 167 | optim. group sep. | 0.541 | 0.964 | 0.353 |
| 167 | Sensitivity fix 0.85 | 0.669 | 0.964 | 0.618 |
| 167 | Sensitivity NT 0.8 | 0.741 | 0.964 | 0.735 |
| 167 | Specificity fix 0.9 | 0.529 | 0.964 | 0.353 |
| 167 | Specificity NT 0.85 | 0.442 | 0.964 | 0.265 |
| 167 | Youden | 0.577 | 0.964 | 0.412 |
| 168 | optim. group sep. | 0.542 | 0.961 | 0.500 |
| 168 | Sensitivity fix 0.85 | 0.626 | 0.961 | 0.588 |
| 168 | Sensitivity NT 0.8 | 0.718 | 0.961 | 0.794 |
| 168 | Specificity fix 0.9 | 0.498 | 0.961 | 0.471 |
| 168 | Specificity NT 0.85 | 0.451 | 0.961 | 0.412 |
| 168 | Youden | 0.572 | 0.961 | 0.529 |
| 169 | optim. group sep. | 0.542 | 0.960 | 0.353 |
| 169 | Sensitivity fix 0.85 | 0.653 | 0.960 | 0.588 |
| 169 | Sensitivity NT 0.8 | 0.721 | 0.960 | 0.765 |
| 169 | Specificity fix 0.9 | 0.526 | 0.960 | 0.324 |
| 169 | Specificity NT 0.85 | 0.462 | 0.960 | 0.265 |
| 169 | Youden | 0.653 | 0.960 | 0.588 |
| 170 | optim. group sep. | 0.542 | 0.963 | 0.441 |
| 170 | Sensitivity fix 0.85 | 0.644 | 0.963 | 0.559 |
| 170 | Sensitivity NT 0.8 | 0.728 | 0.963 | 0.706 |
| 170 | Specificity fix 0.9 | 0.518 | 0.963 | 0.382 |
| 170 | Specificity NT 0.85 | 0.439 | 0.963 | 0.265 |
| 170 | Youden | 0.590 | 0.963 | 0.529 |
| 171 | optim. group sep. | 0.541 | 0.964 | 0.441 |
| 171 | Sensitivity fix 0.85 | 0.670 | 0.964 | 0.588 |
| 171 | Sensitivity NT 0.8 | 0.739 | 0.964 | 0.735 |
| 171 | Specificity fix 0.9 | 0.511 | 0.964 | 0.412 |
| 171 | Specificity NT 0.85 | 0.434 | 0.964 | 0.265 |
| 171 | Youden | 0.613 | 0.964 | 0.559 |
| 172 | optim. group sep. | 0.542 | 0.961 | 0.529 |
| 172 | Sensitivity fix 0.85 | 0.631 | 0.961 | 0.647 |
| 172 | Sensitivity NT 0.8 | 0.738 | 0.961 | 0.824 |
| 172 | Specificity fix 0.9 | 0.515 | 0.961 | 0.471 |
| 172 | Specificity NT 0.85 | 0.460 | 0.961 | 0.412 |
| 172 | Youden | 0.538 | 0.961 | 0.529 |
| 173 | optim. group sep. | 0.540 | 0.965 | 0.441 |
| 173 | Sensitivity fix 0.85 | 0.632 | 0.965 | 0.529 |
| 173 | Sensitivity NT 0.8 | 0.752 | 0.965 | 0.706 |
| 173 | Specificity fix 0.9 | 0.515 | 0.965 | 0.412 |
| 173 | Specificity NT 0.85 | 0.430 | 0.965 | 0.235 |
| 173 | Youden | 0.566 | 0.965 | 0.441 |
| 174 | optim. group sep. | 0.543 | 0.960 | 0.441 |
| 174 | Sensitivity fix 0.85 | 0.637 | 0.960 | 0.559 |
| 174 | Sensitivity NT 0.8 | 0.717 | 0.960 | 0.706 |
| 174 | Specificity fix 0.9 | 0.529 | 0.960 | 0.441 |
| 174 | Specificity NT 0.85 | 0.450 | 0.960 | 0.265 |
| 174 | Youden | 0.570 | 0.960 | 0.529 |
| 175 | optim. group sep. | 0.540 | 0.965 | 0.441 |
| 175 | Sensitivity fix 0.85 | 0.674 | 0.965 | 0.559 |
| 175 | Sensitivity NT 0.8 | 0.747 | 0.965 | 0.735 |
| 175 | Specificity fix 0.9 | 0.499 | 0.965 | 0.382 |
| 175 | Specificity NT 0.85 | 0.432 | 0.965 | 0.294 |
| 175 | Youden | 0.661 | 0.965 | 0.559 |
| 176 | optim. group sep. | 0.540 | 0.966 | 0.441 |
| 176 | Sensitivity fix 0.85 | 0.650 | 0.966 | 0.588 |
| 176 | Sensitivity NT 0.8 | 0.765 | 0.966 | 0.765 |
| 176 | Specificity fix 0.9 | 0.496 | 0.966 | 0.382 |
| 176 | Specificity NT 0.85 | 0.411 | 0.966 | 0.206 |
| 176 | Youden | 0.556 | 0.966 | 0.500 |
| 177 | optim. group sep. | 0.540 | 0.964 | 0.559 |
| 177 | Sensitivity fix 0.85 | 0.644 | 0.964 | 0.676 |
| 177 | Sensitivity NT 0.8 | 0.749 | 0.964 | 0.824 |
| 177 | Specificity fix 0.9 | 0.506 | 0.964 | 0.529 |
| 177 | Specificity NT 0.85 | 0.434 | 0.964 | 0.353 |
| 177 | Youden | 0.578 | 0.964 | 0.588 |
| 178 | optim. group sep. | 0.540 | 0.964 | 0.441 |
| 178 | Sensitivity fix 0.85 | 0.667 | 0.964 | 0.588 |
| 178 | Sensitivity NT 0.8 | 0.735 | 0.964 | 0.765 |
| 178 | Specificity fix 0.9 | 0.514 | 0.964 | 0.382 |
| 178 | Specificity NT 0.85 | 0.436 | 0.964 | 0.294 |
| 178 | Youden | 0.541 | 0.964 | 0.441 |
| 179 | optim. group sep. | 0.540 | 0.966 | 0.441 |
| 179 | Sensitivity fix 0.85 | 0.662 | 0.966 | 0.559 |
| 179 | Sensitivity NT 0.8 | 0.742 | 0.966 | 0.794 |
| 179 | Specificity fix 0.9 | 0.502 | 0.966 | 0.382 |
| 179 | Specificity NT 0.85 | 0.419 | 0.966 | 0.324 |
| 179 | Youden | 0.581 | 0.966 | 0.471 |
| 180 | optim. group sep. | 0.540 | 0.967 | 0.441 |
| 180 | Sensitivity fix 0.85 | 0.660 | 0.967 | 0.588 |
| 180 | Sensitivity NT 0.8 | 0.752 | 0.967 | 0.794 |
| 180 | Specificity fix 0.9 | 0.504 | 0.967 | 0.441 |
| 180 | Specificity NT 0.85 | 0.417 | 0.967 | 0.294 |
| 180 | Youden | 0.560 | 0.967 | 0.500 |
| 181 | optim. group sep. | 0.541 | 0.965 | 0.588 |
| 181 | Sensitivity fix 0.85 | 0.639 | 0.965 | 0.676 |
| 181 | Sensitivity NT 0.8 | 0.749 | 0.965 | 0.824 |
| 181 | Specificity fix 0.9 | 0.499 | 0.965 | 0.500 |
| 181 | Specificity NT 0.85 | 0.449 | 0.965 | 0.382 |
| 181 | Youden | 0.568 | 0.965 | 0.618 |
| 182 | optim. group sep. | 0.540 | 0.966 | 0.441 |
| 182 | Sensitivity fix 0.85 | 0.648 | 0.966 | 0.559 |
| 182 | Sensitivity NT 0.8 | 0.760 | 0.966 | 0.735 |
| 182 | Specificity fix 0.9 | 0.486 | 0.966 | 0.353 |
| 182 | Specificity NT 0.85 | 0.439 | 0.966 | 0.206 |
| 182 | Youden | 0.607 | 0.966 | 0.529 |
| 183 | optim. group sep. | 0.541 | 0.964 | 0.412 |
| 183 | Sensitivity fix 0.85 | 0.655 | 0.964 | 0.588 |
| 183 | Sensitivity NT 0.8 | 0.742 | 0.964 | 0.794 |
| 183 | Specificity fix 0.9 | 0.513 | 0.964 | 0.412 |
| 183 | Specificity NT 0.85 | 0.440 | 0.964 | 0.353 |
| 183 | Youden | 0.601 | 0.964 | 0.500 |
| 184 | optim. group sep. | 0.541 | 0.963 | 0.412 |
| 184 | Sensitivity fix 0.85 | 0.635 | 0.963 | 0.529 |
| 184 | Sensitivity NT 0.8 | 0.742 | 0.963 | 0.735 |
| 184 | Specificity fix 0.9 | 0.510 | 0.963 | 0.324 |
| 184 | Specificity NT 0.85 | 0.451 | 0.963 | 0.294 |
| 184 | Youden | 0.611 | 0.963 | 0.529 |
| 185 | optim. group sep. | 0.540 | 0.965 | 0.412 |
| 185 | Sensitivity fix 0.85 | 0.667 | 0.965 | 0.647 |
| 185 | Sensitivity NT 0.8 | 0.753 | 0.965 | 0.735 |
| 185 | Specificity fix 0.9 | 0.514 | 0.965 | 0.353 |
| 185 | Specificity NT 0.85 | 0.422 | 0.965 | 0.235 |
| 185 | Youden | 0.547 | 0.965 | 0.441 |
| 186 | optim. group sep. | 0.541 | 0.962 | 0.529 |
| 186 | Sensitivity fix 0.85 | 0.615 | 0.962 | 0.559 |
| 186 | Sensitivity NT 0.8 | 0.739 | 0.962 | 0.824 |
| 186 | Specificity fix 0.9 | 0.488 | 0.962 | 0.441 |
| 186 | Specificity NT 0.85 | 0.451 | 0.962 | 0.382 |
| 186 | Youden | 0.490 | 0.962 | 0.441 |
| 187 | optim. group sep. | 0.542 | 0.961 | 0.412 |
| 187 | Sensitivity fix 0.85 | 0.632 | 0.961 | 0.529 |
| 187 | Sensitivity NT 0.8 | 0.726 | 0.961 | 0.735 |
| 187 | Specificity fix 0.9 | 0.505 | 0.961 | 0.353 |
| 187 | Specificity NT 0.85 | 0.447 | 0.961 | 0.265 |
| 187 | Youden | 0.570 | 0.961 | 0.441 |
| 188 | optim. group sep. | 0.541 | 0.964 | 0.441 |
| 188 | Sensitivity fix 0.85 | 0.633 | 0.964 | 0.529 |
| 188 | Sensitivity NT 0.8 | 0.738 | 0.964 | 0.765 |
| 188 | Specificity fix 0.9 | 0.508 | 0.964 | 0.412 |
| 188 | Specificity NT 0.85 | 0.438 | 0.964 | 0.265 |
| 188 | Youden | 0.516 | 0.964 | 0.412 |
| 189 | optim. group sep. | 0.541 | 0.965 | 0.500 |
| 189 | Sensitivity fix 0.85 | 0.645 | 0.965 | 0.588 |
| 189 | Sensitivity NT 0.8 | 0.744 | 0.965 | 0.735 |
| 189 | Specificity fix 0.9 | 0.501 | 0.965 | 0.412 |
| 189 | Specificity NT 0.85 | 0.422 | 0.965 | 0.235 |
| 189 | Youden | 0.569 | 0.965 | 0.500 |
| 190 | optim. group sep. | 0.542 | 0.963 | 0.559 |
| 190 | Sensitivity fix 0.85 | 0.619 | 0.963 | 0.559 |
| 190 | Sensitivity NT 0.8 | 0.746 | 0.963 | 0.824 |
| 190 | Specificity fix 0.9 | 0.491 | 0.963 | 0.441 |
| 190 | Specificity NT 0.85 | 0.442 | 0.963 | 0.412 |
| 190 | Youden | 0.491 | 0.963 | 0.441 |
| 191 | optim. group sep. | 0.540 | 0.964 | 0.559 |
| 191 | Sensitivity fix 0.85 | 0.633 | 0.964 | 0.647 |
| 191 | Sensitivity NT 0.8 | 0.752 | 0.964 | 0.765 |
| 191 | Specificity fix 0.9 | 0.510 | 0.964 | 0.529 |
| 191 | Specificity NT 0.85 | 0.446 | 0.964 | 0.353 |
| 191 | Youden | 0.602 | 0.964 | 0.588 |
| 192 | optim. group sep. | 0.542 | 0.961 | 0.471 |
| 192 | Sensitivity fix 0.85 | 0.627 | 0.961 | 0.559 |
| 192 | Sensitivity NT 0.8 | 0.723 | 0.961 | 0.735 |
| 192 | Specificity fix 0.9 | 0.511 | 0.961 | 0.412 |
| 192 | Specificity NT 0.85 | 0.451 | 0.961 | 0.265 |
| 192 | Youden | 0.558 | 0.961 | 0.471 |
| 193 | optim. group sep. | 0.540 | 0.963 | 0.471 |
| 193 | Sensitivity fix 0.85 | 0.640 | 0.963 | 0.559 |
| 193 | Sensitivity NT 0.8 | 0.738 | 0.963 | 0.735 |
| 193 | Specificity fix 0.9 | 0.520 | 0.963 | 0.412 |
| 193 | Specificity NT 0.85 | 0.433 | 0.963 | 0.265 |
| 193 | Youden | 0.534 | 0.963 | 0.471 |
| 194 | optim. group sep. | 0.540 | 0.966 | 0.441 |
| 194 | Sensitivity fix 0.85 | 0.634 | 0.966 | 0.529 |
| 194 | Sensitivity NT 0.8 | 0.759 | 0.966 | 0.735 |
| 194 | Specificity fix 0.9 | 0.498 | 0.966 | 0.382 |
| 194 | Specificity NT 0.85 | 0.440 | 0.966 | 0.265 |
| 194 | Youden | 0.630 | 0.966 | 0.529 |
| 195 | optim. group sep. | 0.540 | 0.966 | 0.441 |
| 195 | Sensitivity fix 0.85 | 0.662 | 0.966 | 0.618 |
| 195 | Sensitivity NT 0.8 | 0.758 | 0.966 | 0.735 |
| 195 | Specificity fix 0.9 | 0.489 | 0.966 | 0.382 |
| 195 | Specificity NT 0.85 | 0.432 | 0.966 | 0.235 |
| 195 | Youden | 0.633 | 0.966 | 0.559 |
| 196 | optim. group sep. | 0.541 | 0.965 | 0.559 |
| 196 | Sensitivity fix 0.85 | 0.649 | 0.965 | 0.676 |
| 196 | Sensitivity NT 0.8 | 0.749 | 0.965 | 0.765 |
| 196 | Specificity fix 0.9 | 0.500 | 0.965 | 0.500 |
| 196 | Specificity NT 0.85 | 0.444 | 0.965 | 0.382 |
| 196 | Youden | 0.500 | 0.965 | 0.500 |
| 197 | optim. group sep. | 0.540 | 0.966 | 0.471 |
| 197 | Sensitivity fix 0.85 | 0.666 | 0.966 | 0.676 |
| 197 | Sensitivity NT 0.8 | 0.744 | 0.966 | 0.824 |
| 197 | Specificity fix 0.9 | 0.501 | 0.966 | 0.471 |
| 197 | Specificity NT 0.85 | 0.448 | 0.966 | 0.353 |
| 197 | Youden | 0.590 | 0.966 | 0.529 |
| 198 | optim. group sep. | 0.540 | 0.967 | 0.559 |
| 198 | Sensitivity fix 0.85 | 0.667 | 0.967 | 0.735 |
| 198 | Sensitivity NT 0.8 | 0.760 | 0.967 | 0.853 |
| 198 | Specificity fix 0.9 | 0.521 | 0.967 | 0.529 |
| 198 | Specificity NT 0.85 | 0.419 | 0.967 | 0.382 |
| 198 | Youden | 0.575 | 0.967 | 0.559 |
| 199 | optim. group sep. | 0.547 | 0.946 | 0.588 |
| 199 | Sensitivity fix 0.85 | 0.578 | 0.946 | 0.588 |
| 199 | Sensitivity NT 0.8 | 0.660 | 0.946 | 0.706 |
| 199 | Specificity fix 0.9 | 0.577 | 0.946 | 0.588 |
| 199 | Specificity NT 0.85 | 0.513 | 0.946 | 0.559 |
| 199 | Youden | 0.610 | 0.946 | 0.647 |
| 200 | optim. group sep. | 0.540 | 0.968 | 0.500 |
| 200 | Sensitivity fix 0.85 | 0.659 | 0.968 | 0.765 |
| 200 | Sensitivity NT 0.8 | 0.756 | 0.968 | 0.853 |
| 200 | Specificity fix 0.9 | 0.471 | 0.968 | 0.412 |
| 200 | Specificity NT 0.85 | 0.402 | 0.968 | 0.294 |
| 200 | Youden | 0.606 | 0.968 | 0.618 |
| 201 | optim. group sep. | 0.543 | 0.957 | 0.529 |
| 201 | Sensitivity fix 0.85 | 0.594 | 0.957 | 0.588 |
| 201 | Sensitivity NT 0.8 | 0.696 | 0.957 | 0.676 |
| 201 | Specificity fix 0.9 | 0.524 | 0.957 | 0.500 |
| 201 | Specificity NT 0.85 | 0.473 | 0.957 | 0.412 |
| 201 | Youden | 0.569 | 0.957 | 0.588 |
| 202 | optim. group sep. | 0.543 | 0.957 | 0.559 |
| 202 | Sensitivity fix 0.85 | 0.573 | 0.957 | 0.588 |
| 202 | Sensitivity NT 0.8 | 0.702 | 0.957 | 0.676 |
| 202 | Specificity fix 0.9 | 0.532 | 0.957 | 0.559 |
| 202 | Specificity NT 0.85 | 0.470 | 0.957 | 0.412 |
| 202 | Youden | 0.559 | 0.957 | 0.559 |
| 203 | optim. group sep. | 0.543 | 0.957 | 0.529 |
| 203 | Sensitivity fix 0.85 | 0.594 | 0.957 | 0.588 |
| 203 | Sensitivity NT 0.8 | 0.691 | 0.957 | 0.676 |
| 203 | Specificity fix 0.9 | 0.528 | 0.957 | 0.500 |
| 203 | Specificity NT 0.85 | 0.472 | 0.957 | 0.412 |
| 203 | Youden | 0.587 | 0.957 | 0.588 |
| 204 | optim. group sep. | 0.546 | 0.947 | 0.647 |
| 204 | Sensitivity fix 0.85 | 0.573 | 0.947 | 0.735 |
| 204 | Sensitivity NT 0.8 | 0.656 | 0.947 | 0.735 |
| 204 | Specificity fix 0.9 | 0.547 | 0.947 | 0.647 |
| 204 | Specificity NT 0.85 | 0.501 | 0.947 | 0.618 |
| 204 | Youden | 0.608 | 0.947 | 0.735 |
| 205 | optim. group sep. | 0.543 | 0.958 | 0.559 |
| 205 | Sensitivity fix 0.85 | 0.598 | 0.958 | 0.647 |
| 205 | Sensitivity NT 0.8 | 0.702 | 0.958 | 0.676 |
| 205 | Specificity fix 0.9 | 0.539 | 0.958 | 0.559 |
| 205 | Specificity NT 0.85 | 0.483 | 0.958 | 0.471 |
| 205 | Youden | 0.640 | 0.958 | 0.647 |
| 206 | optim. group sep. | 0.545 | 0.954 | 0.559 |
| 206 | Sensitivity fix 0.85 | 0.579 | 0.954 | 0.647 |
| 206 | Sensitivity NT 0.8 | 0.676 | 0.954 | 0.676 |
| 206 | Specificity fix 0.9 | 0.549 | 0.954 | 0.588 |
| 206 | Specificity NT 0.85 | 0.481 | 0.954 | 0.471 |
| 206 | Youden | 0.652 | 0.954 | 0.676 |

ANY REFERENCE TO TABLE 13 SHOULD BE CONSIDERED AS NON-EXISTENT

1-15. (canceled)

16. A method for differentiating in a subject between heart failure and pulmonary disease comprising:
a) determining in a blood, serum or plasma sample of a subject at least the amounts of the three biomarkers OSS2, PC4 and SM23, and the amount of NT-proBNP or BNP, and
b1) calculating a score based on the determined amounts of the three biomarkers and on the amount of BNP or NT-proBNP as referred to in step a), and comparing the, thus, calculated score to a reference score, whereby it is differentiated in a subject between heart failure and pulmonary disease, or
b2) calculating a score based on the determined amounts of the three biomarkers, and comparing the, thus, calculated score to a reference score, and comparing the amount of BNP or NT-proBNP to a reference, whereby it is differentiated in a subject between heart failure and pulmonary disease, wherein SM23 is Sphingomyelin(d18:1/23:1), ii) Sphingomyelin(d18:2/23:0), iii) Sphingomyelin(d17:1/24:1), or iv) a combination of Sphingomyelin(d18:1/23:1), Sphingomyelin(d18:2/23:0) and Sphingomyelin(d17:1/24:1), OSS2 is TAG(C18:1, C18:0, C18:0) and PC4 is Phosphatidylcholine (C16:0 C18:2).

17. The method of claim 16, wherein the subject is suspected to suffer from pulmonary disease or heart failure.

18. The method of claim 16, wherein the subject suffers from shortness of breath (dyspnea).

19. The method of claim 18, wherein the shortness of breath is acute shortness of breath.

20. The method of claim 18, wherein the shortness of breath is chronic shortness of breath.

21. The method of claim 16, wherein the subject is a human subject.

22. The method of claim 16, wherein the amounts of the at least three biomarkers are determined by mass spectrometry (MS).

23. The method according to claim 18, wherein it is differentiated between heart failure and pulmonary disease as the cause of shortness of breath.

24. A diagnostic device for carrying out the method according to claim 16, comprising:
a) an analysing unit comprising at least one detector for at least three biomarkers as set forth in claim 16, wherein said analyzing unit is adapted for determining the amounts of the said biomarkers detected by the at least one detector, and, operatively linked thereto;
b) an evaluation unit comprising a computer comprising tangibly embedded a computer program code for carrying out a comparison of the determined amounts of the at least three biomarkers, and reference amounts and a data base comprising said reference amounts for the said biomarkers, whereby it is differentiated between heart failure and pulmonary disease.

25. Use of i) at least three biomarkers as set forth in claim 16 and of ii) NT-proBNP or BNP in blood, serum or plasma sample of a subject for differentiating in a subject between heart failure and pulmonary disease.

26. Use of i) at least three biomarkers as set forth in claim 16 and of ii) NT-proBNP or BNP in blood, serum or plasma sample of a subject for the preparation of a pharmaceutical and/or diagnostic composition for differentiating in a subject between heart failure and pulmonary disease.
